(19) United States
(12) Patent Application Publication
(10) Pub. No.: US 2024/0158793 A1
(43) Pub. Date: May 16, 2024

(54) REPRESSOR PROTEINS FOR GENE REGULATION AND CRISPR INTERFERENCE

(71) Applicant: Georgia Tech Research Corporation, Atlanta, GA (US)

(72) Inventors: John James Blazeck, Atlanta, GA (US); Andrew J. Kristof, Atlanta, GA (US)

(21) Appl. No.: 18/506,430

(22) Filed: Nov. 10, 2023

Related U.S. Application Data
(60) Provisional application No. 63/424,588, filed on Nov. 11, 2022.

(51) Int. Cl. C12N 15/113; C07K 14/47; C12N 9/22; C12N 15/63

(52) U.S. Cl. CPC C12N 15/113; C07K 14/4703; C12N 9/22; C12N 15/63; C07K 2319/09; C12N 2310/20

(57) ABSTRACT

The present disclosure relates to CRISPR interference (CRISPRi) systems and uses thereof.

### CROSS-REFERENCE TO RELATED APPLICATIONS

This PCT application claims priority to, and the benefit of, U.S. Provisional Patent Application No. 63/424,588, filed Nov. 11, 2022, which is incorporated by reference herein in its entirety.

### STATEMENT REGARDING FEDERALLY SPONSORED RESEARCH

This invention was made with Government Support under Grant No. 1DP2CA280622-01 awarded by the National Institutes of Health. The Government has certain rights in the invention.

### REFERENCE TO SEQUENCE LISTING

The sequence listing submitted on Nov. 10, 2023, as an .XML file entitled “10034-218US1_ST26.xml”,” created on Nov. 8, 2023, and having a file size of 314,914 bytes is hereby incorporated by reference pursuant to 37 C.F.R. § 1.52(e)(5).

### FIELD

The present disclosure relates to CRISPR interference systems and uses thereof.

### BACKGROUND

Being able to control gene expression is essential for biological studies and controlling the function of human cells when engineering them in gene therapy applications. The current gold standard for decreasing gene expression in human cells is to use a dCas9 nuclease fused to a repressor protein that can target a gene's promoter and shut down its expression. However, current CRISPRi limitations include: (1) incomplete gene knockdown that significantly limits CRISPR phenotype screening, (2) sgRNA sequence-dependent repression activity, and (3) variable performance across human cancer cell lines. Therefore, what is needed are novel CRISPR interference systems. The systems, compositions, and methods disclosed herein address these and other needs.

### SUMMARY

The present disclosure provides a CRISPR interference (CRISPRi) system for silencing, reducing, knocking-down, decreasing, and/or eliminating gene expression. The present disclosure also provides an expression vector (including, but not limited to a plasmid, viral vector, a virus, nanoparticle, and/or naked DNA) comprising the CRISPRi system. The present disclosure also provides a cell (including, but not limited to mammalian cells, plant cells, bacterial cells, and/or yeast cell) comprising the CRISPRi system.

In some aspects, disclosed herein is a CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In some aspects, disclosed herein is a CRISPR interference (CRISPRi) system comprising a single guide RNA (sgRNA) and a catalytically inactive nuclease operably fused to a repressor fusion peptide, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some aspects, disclosed herein is an engineered cell comprising a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In some aspect, disclosed herein is an engineered cell comprising a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises three or more repressor domains any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the two or more repressor domains comprise SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, or a fragment thereof.

In some embodiments, the three or more repressor domains comprise SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 114, or a fragment thereof.

In some embodiments, the catalytically inactive nuclease comprises a dCas nuclease selected from a dCas9, dCas12a, and dCas13. In some embodiments, the catalytically inactive nuclease comprises at least 90% sequence identity to SEQ ID NO: 2.

In some embodiments, the repressor fusion peptide is fused to a nuclear localization signal (NLS).

In one aspect, disclosed herein is an expression vector comprising one or more nucleic acids encoding a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In one aspect, disclosed herein is an expression vector comprising one or more nucleic acids encoding a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the one or more nucleic acids encoding the two or more repressor domains comprise SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, or a fragment thereof.

In some embodiments, the one or more nucleic acids encoding the three or more repressor domains comprise SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO:89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 113, or a fragment thereof.

In some embodiments, the one or more nucleic acid encoding the catalytically inactive nuclease comprises at least 90% sequence identity to SEQ ID NO: 1.

In some embodiments, the one or more nucleic acids encodes the two or more repressor fusion peptides fused to a nuclear localization signal.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show the libraries of new repressors.

FIGS. 2A, 2B, 2C, 2D, and 2E show the assessment of the CRISPRi systems using guide RNA (gRNA) tiling, real-time PCR (RT-PCR), and gRNA scaffolds.

FIGS. 3A, 3B, 3C, 3D, 3E, and 3F show the assessment of the CRISPRi systems using cell growth rate assays when targeting essential or nonessential genes and the expression of essential genes using RT-PCR.

FIGS. 4A, 4B, 4C, 4D, and 4E show the evaluation of CRISPRi systems and cell-specific performance determinants across mammalian cell lines.

FIGS. 5A, 5B, and 5C show that truncated MeCP2 variants improve CRISPRi system-mediated gene knockdown across mammalian cell lines.

FIGS. 6A and 6B show that gene silencing is achieved by improved bipartite repressor domains using SV40-eGFP reporter assay with dual targeting gRNA-229/301 (T) in HEK293T cells.

FIGS. 7A, 7B, and 7C show the expression level of CRISPRi systems discovered in screening experiments.

FIGS. 8A, 8B, 8C, 8D, 8E, and 8F show the quantification of growth phenotypes resulting from essential gene knockdown.

FIGS. 9A, 9B, 9C, 9D, 9E, 9F, 9G, and 9H show the characterization of CRISPRi systems in different mammalian cell lines.

FIGS. 10A and 10B show the comparison between MAX secondary effector expression levels.

FIG. 11 shows the fusing of NLS domains to dCas9-ZIM3(KRAB) improves gene knockdown in multiple human cell lines. dCas9-repressor fusions with appended C-terminal NLS domains were transiently transfected in each cell line in 24-well plates, and 72 h later assayed using flow cytometry. eGFP expression is normalized by cells receiving non-targeting sgRNAs. Data denotes the mean expression of three individual cell transfections (cell cultures); error bars represent standard deviations.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

A “control” is an alternative subject or sample used in an experiment for comparison purposes. A control can be “positive” or “negative.”

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100%, or more, increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a Cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules that comprise a Rec3 clamp, as described below.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA).

### CRISPR Interference Systems

Clustered regularly interspaced short palindromic repeats (CRISPR) and CRISPR-associated system (CRISPR/-Cas9) is a popular tool for genome editing. As used herein, genome editing refers to the strategies and techniques for the targeted, specific modification of the genetic information (genome) of living organisms. Genome engineering is a very active field of research because of the wide range of applications, particularly in the areas of human health. For example, genome engineering can be used to alter (e.g., correct or inhibit) a gene carrying a harmful mutation or to explore the function of a gene. One such area of CRISPR genome editing applies to CRISPR interference (CRISPRi) technologies, which refers to a genetic perturbation technique that allows for sequence-specific repression of gene expression in prokaryotic or eukaryotic cells. CRISPRi technologies have been developed to incorporate a catalytically inactive nuclease and a single-guide RNA to repress sequence-specific genes. Further developments of CRISPRi technologies have incorporated repressor proteins, or domains thereof, to enhance gene repression. However, these developments are still limited by (1) incomplete gene knockdown that significantly limits CRISPR phenotype screening, (2) sgRNA sequence-dependent repression activity, and (3) variable performance across human cell lines. Therefore, what is needed is a CRISPRi system that efficiently decreases, reduces, silences, knocks-down, or knocks-out gene expression in a sequence-specific manner in numerous human cell lines while also not being dependent on sgRNA sequences.

Thus, the present disclosure provides a CRISPR interference (CRISPRi) system for silencing, reducing, knocking-down, decreasing, and/or eliminating gene expression. The present disclosure also provides an expression vector (including, but not limited to a plasmid, viral vector, a virus, nanoparticle, and/or naked DNA) comprising the CRISPRi system. The present disclosure also provides a cell (including, but not limited to mammalian cells, plant cells, bacterial cells, and/or yeast cell) comprising the CRISPRi system.

The present disclosure provides CRISPRi systems comprising more than one repressor fusion peptide fused to a catalytically inactive nuclease, such as for example, a dead Cas nuclease (including but not limited to dCas9, dCas12, and dCas13). In some embodiments, the CRISPRi system comprises 2, 3, 4, 5, 6, 7, 8, 9, 10, or more repressor fusion peptides fused to a catalytically inactive nuclease. In some embodiments, the CRISPRi system comprises a bipartite repressor fusion peptide fused to a catalytically inactive nuclease. In some embodiments, the CRISPRi system comprises a tripartite repressor fusion peptide fused to a catalytically inactive nuclease.

As used herein, a “bipartite repressor fusion peptide” refers to a system, composition, or biological matter comprising two distinct repressor domains fused together by at least one linker. In some embodiments, the two distinct repressor domains are the same. In some embodiments, the two distinct repressor domains are different. In some embodiments, at least one peptide of the bipartite repressor fusion peptides comprises a Kruppel-associated box (KRAB) domain, a NcoR/SMRT interaction domain (NID), or a combination thereof.

As used herein, a “tripartite repressor fusion peptide” refers to a system, composition, or biological matter comprising three distinct repressor domains fused together by at least one linker. In some embodiments, the three distinct repressor domains are the same. In some embodiments, two out of three distinct repressor domains are the same. In some embodiments, the three distinct repressor domains are different. In some embodiments, two of out three distinct repressor domains are different. In some embodiments, at least one peptide of the tripartite repressor fusion peptides comprises a Kruppel-associated box (KRAB) domain, a NcoR/SMRT interaction domain (NID), or a combination thereof.

In some aspects, disclosed herein is a CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In some aspects, disclosed herein is a CRISPR interference (CRISPRi) system comprising a single guide RNA (sgRNA) and a catalytically inactive nuclease operably fused to a repressor fusion peptide, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some aspects, disclosed herein is an engineered cell comprising a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In some aspect, disclosed herein is an engineered cell comprising a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises three or more repressor domains any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the engineered cell comprises a mammalian cell, a bacterial cell, a plant cell, a yeast cell, or a cancer cell.

In some embodiments, the two or more repressor domains comprise SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 114, or a fragment thereof. In some embodiments, the two or more repressor domains comprise SEQ ID NO: 114, SEQ ID NO: 38, SEQ ID NO: 40, SEQ ID NO: 42, SEQ ID NO: 44, or a fragment thereof.

In some embodiments, the three or more repressor domains comprise SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, or a fragment thereof. In some embodiments, the three or more repressor domains comprise SEQ ID NO: 64.

In some embodiments, the catalytically inactive nuclease is fused to two repressor fusion peptides comprising KOX1(KRAB)-MeCP2, ZIM3(KRAB)-MeCP2, KOX1(KRAB)-MeCP2(t), ZIM3(KRAB)-MAX, KRBOX1(KRAB)-MAX, KOX1(KRAB)-MAX, ZIM3(KRAB)-IRF2BP1, ZIM3(KRAB)-ZIM3(KRAB), KRBOX1(KRAB)-CTCF, ZIM3(KRAB)-ZNF554, KRBOX1(KRAB)-MeCP2, ZIM3(KRAB)-RYBP, ZIM3(KRAB)-KLF10, KRBOX1(KRAB)-ZIM3(KRAB), or a variation thereof.

In some embodiments, the catalytically inactive nuclease is fused to three repressor fusion peptides comprising ZIM3(KRAB)-MAX-MeCP2(t), KOX1(KRAB)-MeCP2(t)-MeCP2(t), KOX1(KRAB)-MeCP2(t)-KOX1(KRAB), ZIM3(KRAB)-MAX-IRF2BP1, KOX1(KRAB)-MeCP2(t)-ZNF264(KRAB), KRBOX1(KRAB)-MAX-MeCP2(t), ZIM3(KRAB)-MeCP2-RYBP, ZIM3(KRAB)-MAX-ZNF554(KRAB), ZIM3(KRAB)-MAX-KOX1(KRAB), ZIM3(KRAB)-MeCP2-KRBOX1, KRBOX1(KRAB)-MAX-ZIM3(KRAB), ZIM3)KRAB)-MeCP2-MeCP2(t), ZIM3(KRAB)-MAX-ZNF264(KRAB), ZIM3(KRAB)-MeCP2-ZIM3(KRAB), KRBOX1(KRAB)-MAX-MeCP2, KRBOX1(KRAB)-MAX-ZNF554(KRAB), ZIM3(KRAB)-MeCP2-IRF2BP1, ZIM3(KRAB)-MeCP2-IRF2BP1, ZIM3(KRAB)-MAX-CTCF, KOX1(KRAB)-MeCP2(t)-SCMH1, ZIM3(KRAB)-MeCP2-KOX1(KRAB), KOX1(KRAB)-MeCP2(t)-RYBP, KRBOX1(KRAB)-MAX-MGA, KRBOX1(KRAB)-MAX-ZNF264(KRAB), or ZIM3(KRAB)-MAX-ZIM3(KRAB).

In some embodiments, the catalytically inactive nuclease is fused to 4, 5, 6, 7, 8, 9, 10, 11, 12, 13, 14, 15, 16, 17, 18, or 19 repressor fusion peptides selected from KOX1, KRBOX1, ZIM3, MAX, MeCP2t, MeCP2, TRIM28, RYBP, CBX1, SCMH1, CTCF, REST, MGA, KLF10, IRF2BP1, IKZF5, RCOR1, ZNF554, and ZNF264.

A nuclear localization signal (NLS) is an amino acid sequence that mediates the transport of protein designated to enter into the nucleus. It has been demonstrated that nuclear and non-nuclear proteins are imported into the nucleus when fused to an NLS. Thus, in some embodiments, the repressor fusion peptide (comprising either two or three repressor domains) is fused to a nuclear localization signal (NLS) comprising SEQ ID NO: 159, SEQ ID NO: 163, SEQ ID NO: 167, SEQ ID NO: 171, SEQ ID NO: 175, SEQ ID NO: 179, SEQ ID NO: 183, SEQ ID NO: 185, SEQ ID NO: 187, SEQ ID NO: 191, or a fragment thereof. In some embodiments, the repressor fusion peptide fused to a NLS comprises SEQ ID NO: 159, SEQ ID NO: 171, or a fragment thereof.

The structure for Cas molecules was determined when bound in complex with a gRNA and double-stranded DNA target, in an active (DNA cleavage product state) and inactive (nonproductive state) conformation. This allowed for rational design of enzymes with different properties that facilitate better gene editing. The Cas nucleases disclosed herein have been mutated within the catalytic domains to be inactive, such that the Cas nuclease lacks endonuclease activity, but still the sgRNA and the promoter of a target gene sequence.

In some embodiments, the catalytically inactive nuclease comprises a dead Cas (dCas) nuclease selected from a dCas9, dCas12a, and dCas13. In some embodiments, the catalytically inactive nuclease comprises at least 50% sequence identity to SEQ ID NO: 2. In some embodiments, the catalytically inactive nuclease comprises at least 60% sequence identity to SEQ ID NO: 2. In some embodiments, the catalytically inactive nuclease comprises at least 70% sequence identity to SEQ ID NO: 2. In some embodiments, the catalytically inactive nuclease comprises at least 80% sequence identity to SEQ ID NO: 2. In some embodiments, the catalytically inactive nuclease comprises at least 90% sequence identity to SEQ ID NO: 2. In some embodiments, the catalytically inactive nuclease comprises at least 95% sequence identity to SEQ ID NO: 2. In some embodiments, the catalytically inactive nuclease comprises at least 99% sequence identity to SEQ ID NO: 2. In some embodiments, the catalytically inactive nuclease comprises SEQ ID NO: 2, or a fragment thereof.

In some embodiments, the sgRNA comprises SEQ ID NO: 133-155, or a fragment thereof, incorporated into a sgRNA scaffold comprising SEQ ID NO: 131 or SEQ ID NO: 132, or a fragment thereof. In some embodiments, the sgRNA targets at a transcriptional start site (TSS) of a gene in a cell. In some embodiments, the sgRNA targets away from a transcriptional start site (TSS) of a gene in a cell.

In some embodiments, the CRISPRi system further comprises a first linker, second linker, and/or a third linker. In some embodiments, the first linker fuses the catalytically inactive nuclease to the first repressor peptide, the second linker fuses the first repressor peptide to the second repressor peptide, and/or the third linker fuses the second repressor peptide to the third repressor peptide. In some embodiments, the first linker, the second linker, and/or third linker are the same. In some embodiments, the first linker and second linker are the same. In some embodiments, the second linker and third linker are the same. In some embodiments, the first linker and third linker are the same. In some embodiments, the first linker, the second linker, and/or third linker are different. In some embodiments, the first linker and second linker are different. In some embodiments, the second linker and third linker are different. In some embodiments, the first linker and third linker are different. In some embodiments, the first linker, the second linker, and/or third linker comprise at least 70% sequence identity of SEQ ID NO: 4, 6, or 8. In some embodiments, the first linker, the second linker, and/or third linker comprise at least 80% sequence identity of SEQ ID NO: 4, 6, or 8. In some embodiments, the first linker, the second linker, and/or third linker comprise at least 90% sequence identity of SEQ ID NO: 4, 6, or 8. In some embodiments, the first linker, the second linker, and/or third linker comprise at least 95% sequence identity of SEQ ID NO: 4, 6, or 8. In some embodiments, the first linker, the second linker, and/or third linker comprise at least 99% sequence identity of SEQ ID NO: 4, 6, or 8. In some embodiments, the first linker, the second linker, and/or third linker comprises SEQ ID NO: 4, 6, or 8.

### Expression Vectors

In one aspect, disclosed herein is an expression vector comprising one or more nucleic acids encoding a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In one aspect, disclosed herein is an expression vector comprising one or more nucleic acids encoding a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the expression vector comprises a plasmid or a virus or viral vector. A plasmid, virus, or a viral vector is capable of extrachromosomal replication or, optionally, can integrate into the host genome. As used herein, the term “integrated” used in reference to an expression vector (e.g., a plasmid, virus, or viral vector) means the expression vector, or a portion thereof, is incorporated (physically inserted or ligated) into the chromosomal DNA of a host cell. As used herein, a “plasmid” refers to a small circular DNA molecule derived from bacteria or other microscopic organisms. Plasmids are physically separate from chromosomal DNA and replicate independently once inside the host organism. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Non-limiting examples of viruses and viral vectors include adenovirus, lentivirus, retrovirus, adeno-associated viruses, retrovirus, and large payload viral vectors. It has been contemplated that the one or more nucleic acids encoding the CRISPRi system can be inserted into a single expression vector or can be separated into two or more expression vectors. Thus, the CRISPRi system disclosed herein can be designed within any number of expression vectors deemed fit to produce the desired effect of gene repression. In some embodiments, the expression vector encoding a CRISPRi system comprises naked DNA or is comprised in a nanoparticle (e.g., liposomal vesicle, porous silicon nanoparticle, gold-DNA conjugate particle, polyethyleneimine polymer particle, cationic peptides, etc.).

In some embodiments, the one or more nucleic acids encodes the two or more repressor fusion peptides comprising SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, or a fragment thereof

In some embodiments, the one or more nucleic acids encodes the three or more repressor fusion peptide comprising SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO:89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 113, or a fragment thereof.

In some embodiments, the one or more nucleic acids encoding the catalytically inactive nuclease comprises at least 50% sequence identity of SEQ ID NO: 1. In some embodiments, the one or more nucleic acids encoding the catalytically inactive nuclease comprises at least 60% sequence identity of SEQ ID NO: 1. In some embodiments, the one or more nucleic acids encoding the catalytically inactive nuclease comprises at least 70% sequence identity of SEQ ID NO: 1. In some embodiments, the one or more nucleic acids encoding the catalytically inactive nuclease comprises at least 80% sequence identity of SEQ ID NO: 1. In some embodiments, the one or more nucleic acids encoding the catalytically inactive nuclease comprises at least 90% sequence identity of SEQ ID NO: 1. In some embodiments, the one or more nucleic acids encoding the catalytically inactive nuclease comprises at least 95% sequence identity of SEQ ID NO: 1. In some embodiments, the one or more nucleic acids encoding the catalytically inactive nuclease comprises at least 99% sequence identity of SEQ ID NO: 1. In some embodiments, the one or more nucleic acid encoding the catalytically inactive nuclease comprises SEQ ID NO: 1, or a fragment thereof.

In some embodiments, the expression vector further comprises the first linker, second linker, and/or third linker of any preceding aspect.

In some embodiments, the one or more nucleic acids encode the sgRNA comprising SEQ ID NO: 133-155, or a fragment thereof, incorporated into a sgRNA scaffold comprising SEQ ID NO: 131 or SEQ ID NO: 132, or a fragment thereof.

Methods of Decreasing and/or Silencing Gene Expression

In one aspect, disclosed herein is a method of decreasing gene expression, the method comprising administering to a host a CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

In some embodiments, the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the method of decreasing gene expression comprises the two or more repressor domains comprising SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 114, or a fragment thereof. In a preferred embodiment, the method of decreasing gene expression comprises the two or more repressor domains comprising SEQ ID NO: 114, SEQ ID NO: 38, SEQ ID NO: 40, SEQ ID NO: 42, SEQ ID NO: 44, or a fragment thereof.

In some embodiments, the method of decreasing gene expression comprises the three or more repressor domains comprising SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 114, or a fragment thereof. In a preferred embodiment, the method of decreasing gene expression comprises the three or more repressor domains comprising SEQ ID NO: 64.

In some embodiments, the method of decreasing gene expression comprises the catalytically inactive nuclease is fused to two repressor fusion peptides comprising KOX1(KRAB)-MeCP2, ZIM3(KRAB)-MeCP2, KOX1(KRAB)-MeCP2(t), ZIM3(KRAB)-MAX, KRBOX1(KRAB)-MAX, KOX1(KRAB)-MAX, ZIM3(KRAB)-IRF2BP1, ZIM3(KRAB)-ZIM3(KRAB), KRBOX1(KRAB)-CTCF, ZIM3(KRAB)-ZNF554, KRBOX1(KRAB)-MeCP2, ZIM3(KRAB)-RYBP, ZIM3(KRAB)-KLF10, KRBOX1(KRAB)-ZIM3(KRAB), or a variation thereof.

In some embodiments, the method of decreasing gene expression comprises the catalytically inactive nuclease is fused to three repressor fusion peptides comprising ZIM3(KRAB)-MAX-MeCP2(t), KOX1(KRAB)-MeCP2(t)-MeCP2(t), KOX1(KRAB)-MeCP2(t)-KOX1(KRAB), ZIM3(KRAB)-MAX-IRF2BP1, KOX1(KRAB)-MeCP2(t)-ZNF264(KRAB), KRBOX1(KRAB)-MAX-MeCP2(t), ZIM3(KRAB)-MeCP2-RYBP, ZIM3(KRAB)-MAX-ZNF554(KRAB), ZIM3(KRAB)-MAX-KOX1(KRAB), ZIM3(KRAB)-MeCP2-KRBOX1, KRBOX1(KRAB)-MAX-ZIM3(KRAB), ZIM3)KRAB)-MeCP2-MeCP2(t), ZIM3(KRAB)-MAX-ZNF264(KRAB), ZIM3(KRAB)-MeCP2-ZIM3(KRAB), KRBOX1(KRAB)-MAX-MeCP2, KRBOX1(KRAB)-MAX-ZNF554(KRAB), ZIM3(KRAB)-MeCP2-IRF2BP1, ZIM3(KRAB)-MeCP2-IRF2BP1, ZIM3(KRAB)-MAX-CTCF, KOX1(KRAB)-MeCP2(t)-SCMH1, ZIM3(KRAB)-MeCP2-KOX1(KRAB), KOX1(KRAB)-MeCP2(t)-RYBP, KRBOX1(KRAB)-MAX-MGA, KRBOX1(KRAB)-MAX-ZNF264(KRAB), or ZIM3(KRAB)-MAX-ZIM3(KRAB).

In some embodiments, the method of decreasing gene expression comprises the catalytically inactive nuclease is fused to 4, 5, 6, 7, 8, 9, 10, 11, 12, 13, 14, 15, 16, 17, 18, or 19 repressor fusion peptides selected from KOX1, KRBOX1, ZIM3, MAX, MeCP2t, MeCP2, TRIM28, RYBP, CBX1, SCMH1, CTCF, REST, MGA, KLF10, IRF2BP1, IKZF5, RCOR1, ZNF554, and ZNF264.

In some embodiments, the host comprises a cell, a mammal, or a human. In some embodiments, the cell comprises a mammalian cell, a bacterial cell, a plant cell, a yeast cell, or a cancer cell.

In some embodiments, the method of decreasing gene expression comprises forming a nuclease-sgRNA complex, wherein the catalytically inactive nuclease is fused to two or more, or three or more repressor fusion peptides, the nuclease then binds to the sgRNA, and the nuclease-sgRNA complex targets and binds at a promoter of a target gene sequence. In some embodiments, the nuclease-sgRNA complex binds at a transcriptional start site (TSS) of the target gene. In some embodiments, the nuclease-sgRNA complex binds away from the TSS of the target gene.

In some embodiments, the two or more, or three or more repressor fusion peptides enhance silencing, decreasing, knocking-down, or reducing the gene expression of the target gene. In some embodiments, the method of decreasing gene expression further comprises treating and/or preventing a disease or disorder.

Methods of Treating and/or Preventing Disease

In one aspect, disclosed herein is a method of treating and/or preventing a disease or disorder in a subject, the method comprising administering to a subject a CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof, and wherein the CRISPRi system silences, decreases, knocks-down, knocks-out, or reduces gene expression of a target gene.

In some embodiments, the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

In some embodiments, the method of treating and/or preventing a disease or disorder comprises the two or more repressor domains comprising SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 114, or a fragment thereof. In a preferred embodiment, the method of treat and/or preventing a disease or disorder comprises the two or more repressor domains comprising SEQ ID NO: 114, SEQ ID NO: 38, SEQ ID NO: 40, SEQ ID NO: 42, SEQ ID NO: 44, or a fragment thereof.

In some embodiments, the method of treating and/or preventing a disease or disorder comprises the three or more repressor domains comprising SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, or a fragment thereof. In a preferred embodiment, the method of treating and/or preventing a disease or disorder comprises the three or more repressor domains comprising SEQ ID NO: 64, or a fragment.

In some embodiments, the method of treating and/or preventing a disease or disorder comprises the catalytically inactive nuclease is fused to two repressor fusion peptides comprising KOX1(KRAB)-MeCP2, ZIM3(KRAB)-MeCP2, KOX1(KRAB)-MeCP2(t), ZIM3(KRAB)-MAX, KRBOX1(KRAB)-MAX, KOX1(KRAB)-MAX, ZIM3(KRAB)-IRF2BP1, ZIM3(KRAB)-ZIM3(KRAB), KRBOX1(KRAB)-CTCF, ZIM3(KRAB)-ZNF554, KRBOX1(KRAB)-MeCP2, ZIM3(KRAB)-RYBP, ZIM3(KRAB)-KLF10, KRBOX1(KRAB)-ZIM3(KRAB), or a variation thereof.

In some embodiments, the method of treating and/or preventing a disease or disorder comprises the catalytically inactive nuclease is fused to three repressor fusion peptides comprising ZIM3(KRAB)-MAX-MeCP2(t), KOX1(KRAB)-MeCP2(t)-MeCP2(t), KOX1(KRAB)-MeCP2(t)-KOX1(KRAB), ZIM3(KRAB)-MAX-IRF2BP1, KOX1(KRAB)-MeCP2(t)-ZNF264(KRAB), KRBOX1(KRAB)-MAX-MeCP2(t), ZIM3(KRAB)-MeCP2-RYBP, ZIM3(KRAB)-MAX-ZNF554(KRAB), ZIM3(KRAB)-MAX-KOX1(KRAB), ZIM3(KRAB)-MeCP2-KRBOX1, KRBOX1(KRAB)-MAX-ZIM3(KRAB), ZIM3)KRAB)-MeCP2-MeCP2(t), ZIM3(KRAB)-MAX-ZNF264(KRAB), ZIM3(KRAB)-MeCP2-ZIM3(KRAB), KRBOX1(KRAB)-MAX-MeCP2, KRBOX1(KRAB)-MAX-ZNF554(KRAB), ZIM3(KRAB)-MeCP2-IRF2BP1, ZIM3(KRAB)-MeCP2-IRF2BP1, ZIM3(KRAB)-MAX-CTCF, KOX1(KRAB)-MeCP2(t)-SCMH1, ZIM3(KRAB)-MeCP2-KOX1(KRAB), KOX1(KRAB)-MeCP2(t)-RYBP, KRBOX1(KRAB)-MAX-MGA, KRBOX1(KRAB)-MAX-ZNF264(KRAB), or ZIM3(KRAB)-MAX-ZIM3(KRAB).

In some embodiments, the method of treating and/or preventing a disease or disorder comprises the catalytically inactive nuclease is fused to 4, 5, 6, 7, 8, 9, 10, 11, 12, 13, 14, 15, 16, 17, 18, or 19 repressor fusion peptides selected from KOX1, KRBOX1, ZIM3, MAX, MeCP2t, MeCP2, TRIM28, RYBP, CBX1, SCMH1, CTCF, REST, MGA, KLF10, IRF2BP1, IKZF5, RCOR1, ZNF554, and ZNF264.

In some embodiments, the method of treating and/or preventing a disease or disorder comprises forming a nuclease-sgRNA complex, wherein the catalytically inactive nuclease is fused to two or more, or three or more repressor fusion peptides, the nuclease then binds to the sgRNA, and the nuclease-sgRNA complex targets and binds at a promoter of a target gene sequence. In some embodiments, the nuclease-sgRNA complex binds at a transcriptional start site (TSS) of the target gene. In some embodiments, the nuclease-sgRNA complex binds away from the TSS of the target gene.

In some embodiments, the target gene includes, but is not limited to an overexpressed gene, an oncogene, a mutant gene encoding a protein, and a gene encoding a misfolded protein. In some embodiments, the subject is a human. In some embodiments, the subject has a genetic disorder. In some embodiments, the subject has cancer.

It should be understood the CRISPRi system can be administered as a therapeutic composition deemed fit to generate the desired effect of silencing, decreasing, knocking-down, or reducing gene expression. Thus, the CRISPRi system can be administered in a pharmaceutically acceptable carrier, wherein the CRISPRi system is incorporated in a vector, a cell, or as a naked system. The CRISPRi composition may be administered in such amounts, time, and route deemed necessary in order to achieve the desired result. The exact amount of the CRISPRi composition will vary from subject to subject, depending on the species, age, and general condition of the subject, the severity of the disease or disorder the particular CRISPRi composition, its mode of administration, its mode of activity, and the like. The CRISPRi composition is preferably formulated in dosage unit form for ease of administration and uniformity of dosage. It will be understood, however, that the total daily usage of the CRISPRi composition will be decided by the attending physician within the scope of sound medical judgment. The specific therapeutically effective dose level for any particular subject will depend upon a variety of factors including the disease or disorder being treated and the severity of the symptoms associated with the disease or disorder; the activity of the CRISPRi composition employed; the specific CRISPRi composition employed; the age, body weight, general health, sex and diet of the patient; the time of administration, route of administration, and rate of excretion of the specific CRISPRi composition employed; the duration of the treatment; drugs used in combination or coincidental with the specific CRISPRi composition employed; and like factors well known in the medical arts.

The CRISPRi composition may be administered by any route. In some embodiments, the CRISPRi composition is administered via a variety of routes, including oral, intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the CRISPRi composition (e.g., its stability in the environment of the body of the host/subject), the condition of the subject (e.g., whether the subject is able to tolerate oral administration), etc.

The exact amount of CRISPRi composition required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is CRISPRi system of any preceding aspect can be added to a pharmaceutically acceptable carrier selected from an excipient, a diluent, a salt, a buffer, a stabilizer, a lipid, an emulsion, a nanoparticle, and a cream. One or more active agents (e.g. CRISPRi system) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the CRISPRi composition is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the CRISPRi composition is administered daily. In some embodiments, the CRISPRi composition is administered every day, every 2 days, every 3 days, every 4 days, every 5 days, every 6 days, every 7 days, or more. In some embodiments, the CRISPRi composition is administered every week, every 2 weeks, every 3 weeks, every 4 weeks, or more. In some embodiments, the CRISPRi composition is administered every month, every 2 months, every 3 months, every 4 months, every 5 months, every 6 months, every 7 months, every 8 months, every 9 months, every 10 months, every 11 months, every 12 months, or more. In some embodiments, the CRISPRi composition is administered every year, every 2 years, every 3 years, every 4 years, every 5 years, or more.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Engineering Next-Generation CRISPRi Repressors for Highly Efficient Mammalian Gene Regulation

CRISPR interference (CRISPRi), the repurposing of the RNA-guided endonuclease dCas9 as a programmable transcriptional repressor, is a powerful genetic tool enabling highly specific repression (knockdown) of gene expression. Despite the system's adoption, CRISPRi platforms still suffer from incomplete knockdown and significant performance variability across cell lines and gene targets. The disclosure herein describes the discovery and characterization of exceptionally potent repressor domain fusions that offer best-in-class gene knockdown efficacy across diverse mammalian cell lines. It is also established here that these variants' best-in-class capability to silence target genes, investigate cellular determinants that control performance while demonstrating enhanced function across a panel of diverse cells lines, and demonstrate that novel truncations of the MeCP2 repressor domain results in vastly improved gene knockdown efficiency.

The ability to reduce or silence gene expression is vital for performing robust whole-genome genetic screens, discovering non-coding transcriptional regulatory motifs, and tuning cellular function in mammalian cells. CRISPR interference (CRISPRi) has emerged as a powerful method enabling site-specific transcriptional repression. The CRISPRi system typically employs two components: 1) a fusion protein combining catalytically dead Cas9 (dCas9) with one or more transcriptional repressor domains that recruit regulatory co-factors natively expressed in mammalian cells, and 2) a single guide RNA (sgRNA) that recognizes DNA sequences through base-pair complementarity, leading the dCas9-repressor fusion to DNA loci with high specificity. When directed toward a target gene promoter, the CRISPRi repressor induces local epigenetic remodeling resulting in reduced gene expression. CRISPRi has proved effective for a broad range of applications, including discovering networks regulating cellular metabolism and signaling, perturbing disease markers in neurons, investigating signaling in primary human T cells, and interrogating genetic vulnerabilities in cancer cells.

CRISPRi platforms possess several advantages over nuclease-active CRISPR-Cas9 systems, which rely on targeted double-stranded DNA breaks within coding regions to eliminate functional protein expression. CRISPRi does induce DNA damage or activate endogenous DNA repair (or apoptotic) pathways, both of which can confound large-scale screens, particularly in sensitive hosts such as stem cells or when targeting high copy number genomic loci. Similarly, whereas Cas9-mediated gene knockouts are irreversible and can often generate cell subpopulations with in-frame indels, partial knockouts yielding fully functional proteins, or initiate nonsense-associated alternative splicing, CRISPRi enables more homogenous, and reversible, gene expression control. These properties can allow for titratable gene expression to map phenotypes to precise levels of individual gene knockdown. CRISPRi systems also permit mapping of regulatory elements and interrogation of non-coding RNAs.

Despite these advantages, several technical limitations hinder the utility of CRISPRi platforms, including poor or moderate knockdown efficiency of targeted genes, widespread functional variance across cell lines or lineages, and notable sgRNA stochasticity. Originally, CRISPRi platforms used only the dCas9 protein directed to a gene's transcription start site to sterically block RNA polymerase passage, but today, almost all CRISPRi platforms utilize fusions of dCas9 with the Kruppel-associated box (KRAB) domain from the human protein KOX1 (ZNF10). This domain, the first functionally characterized CRISPRi repressor, is traditionally known as KRAB but here called KOX1(KRAB) to prevent ambiguity. Previous work has shown that CRISPRi knockdown efficiency can be improved by combining KOX1(KRAB) with additional repressor domains, most notably methyl-CpG binding protein 2 (MeCP2). Also, recent reports showed that alternative KRAB domains from other human proteins, notably ZIM3(KRAB), confer improved gene silencing. Despite the relative outperformance of “gold standard repressors” KOX1(KRAB)-MeCP2 and ZIM3(KRAB) compared to KOX1(KRAB), the associated CRISPRi platforms can still suffer from inefficient knockdown and variable performance across cell lines and gene targets.

Herein, these challenges are addressed by assembling and screening combinatorial libraries of repressor domains to identify high-efficacy variants. Several novel CRISPRi systems were created boasting the highest gene knockdown efficiency reported to-date. The superior performance of these dCas9-repressor fusions for characterizing gene-phenotype relationships in cancer cells and silencing gene expression was demonstrated in a broad panel of mammalian cell lines. Finally, functional domains of MeCP2 were explored, and a truncated MeCP2 domain was identified that, when fused to KRAB domains, significantly improves CRISPRi activity compared to the canonical MeCP2 repressor.

### Results

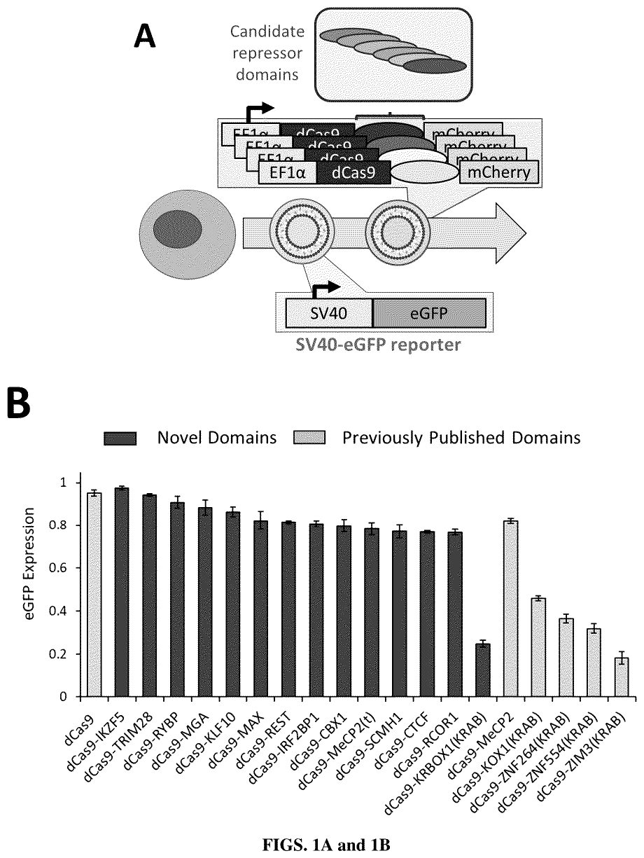

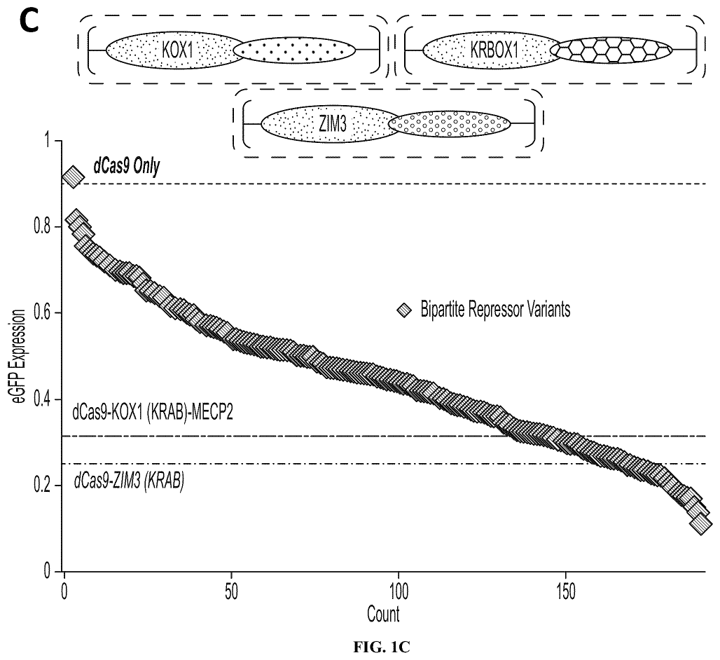

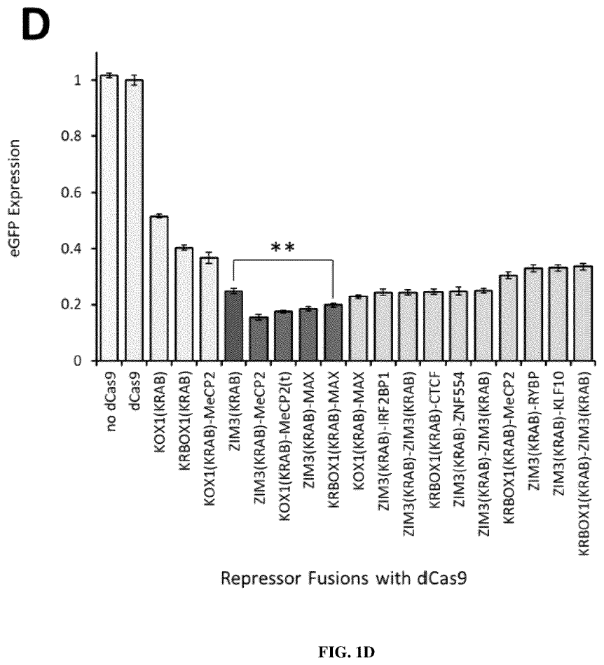

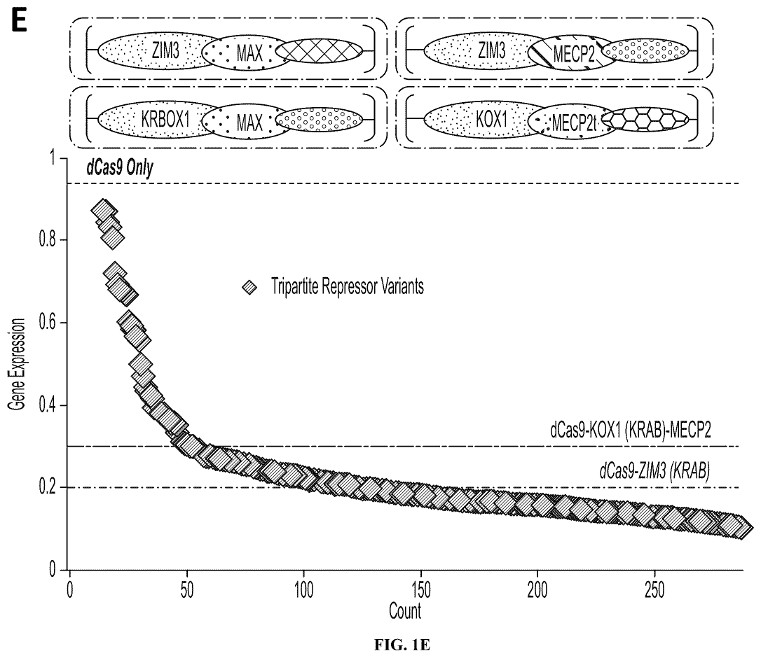

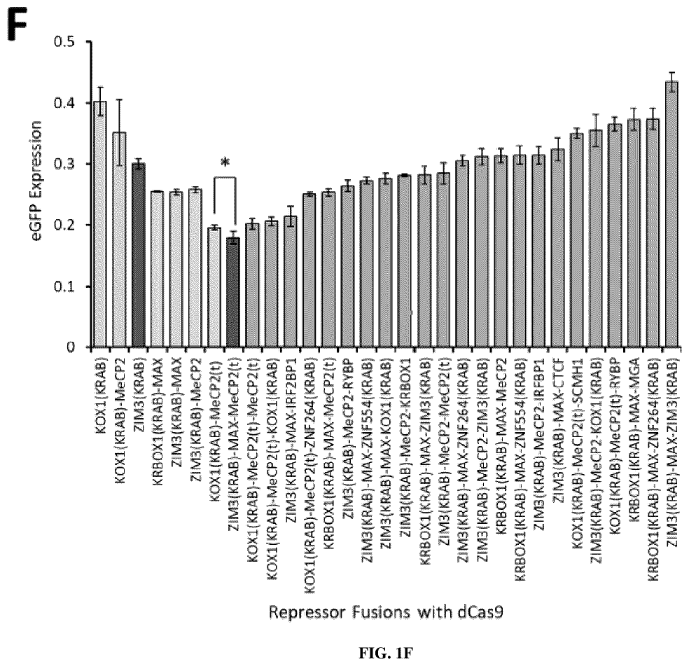

Screening and Characterizing Novel, Best-in-Class CRISPRi Repressors. To design improved CRISPRi systems, putative CRISPRi-compatible transcriptional repressor domains were first selected from a recent tiling library, in which several non-KRAB repressor domains from human proteins were described that had comparable or stronger reported activity than MeCP2, the partner of KOX1(KRAB) in the canonical dCas9-KOX1(KRAB)-MeCP2 CRISPRi system. Because these repressor domains were not tested for their ability to mediate transcriptional repression in the context of a CRISPRi system, i.e., when fused to dCas9, 11 high-confidence domains were first selected and tested for their utility for transcriptional repression in a CRISPRi system using a reporter assay in HEK293T cells (FIG. 1A). Three additional transcriptional regulatory proteins (RCOR1, IKZF5, MAX) were also noticed to predict interactions with these putative repressors or previously described domains, so it was contemplated that they also are paired with dCas9 for highly potent CRISPR-mediated gene knockdown.

Each of the 14 candidate repressor domains were fused to the C-terminus of dCas9, recruited each repressor to two distinct sites on an SV40 promoter regulating expression of enhanced green fluorescent protein (eGFP), and measured resultant eGFP expression levels using flow cytometry. All candidate dCas9-repressor fusions exhibited improved gene knockdown compared to dCas9 alone, and several domains (e.g., CTCF or SCMH1) exhibited comparable activity to MeCP2 when fused to dCas9. Interestingly, the 80AA truncated MeCP2 domain (referred to here as MeCP2(t) for clarity) achieved similar levels of gene knockdown compared to the full-length MeCP2 repressor domain (FIG. 1A). In addition, dCas9-KRBOX1(KRAB) achieved significantly improved eGFP knockdown compared to dCas9-KOX1(KRAB), further highlighting the strong transcriptional repressor activity highly conserved across the 350+ KRAB domains encoded in human proteins.

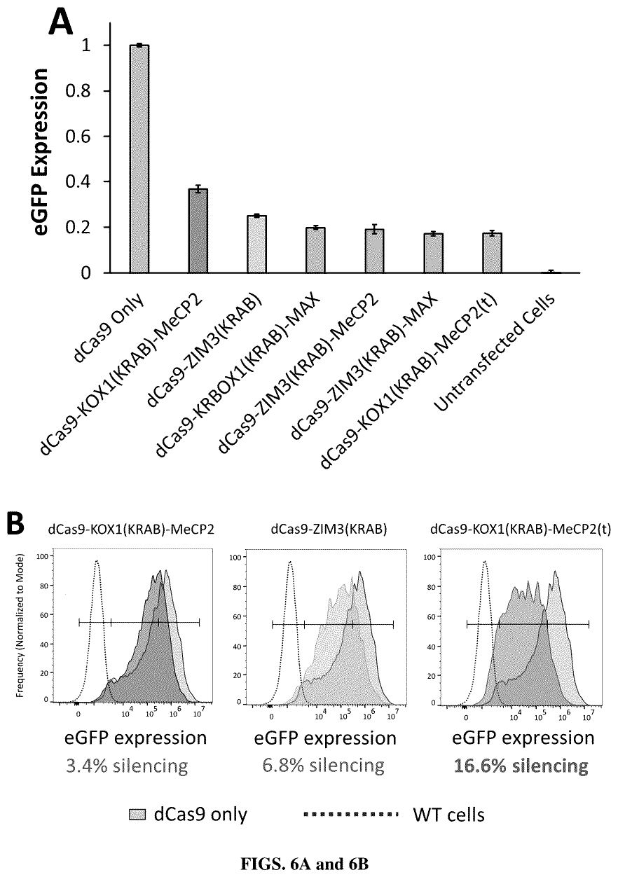

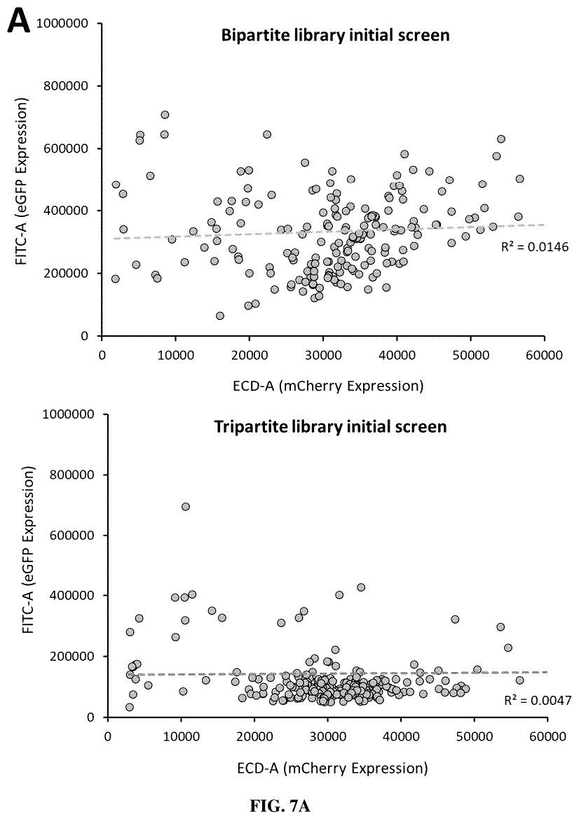

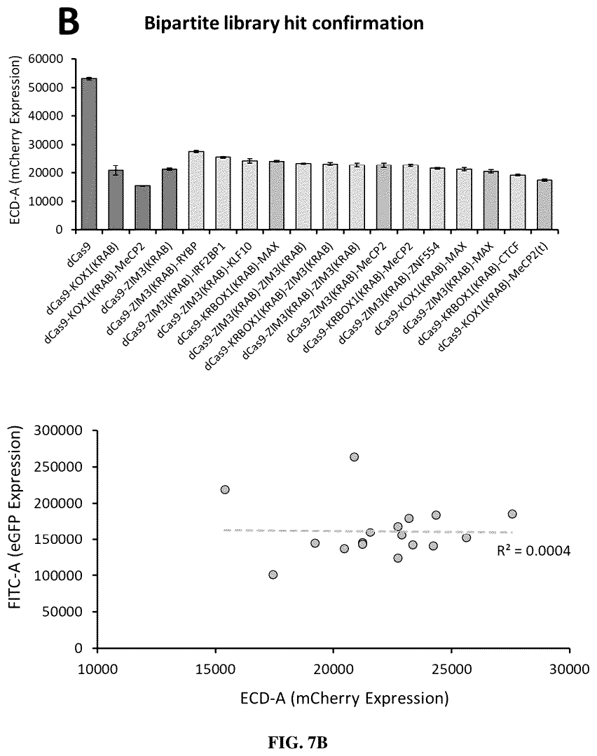

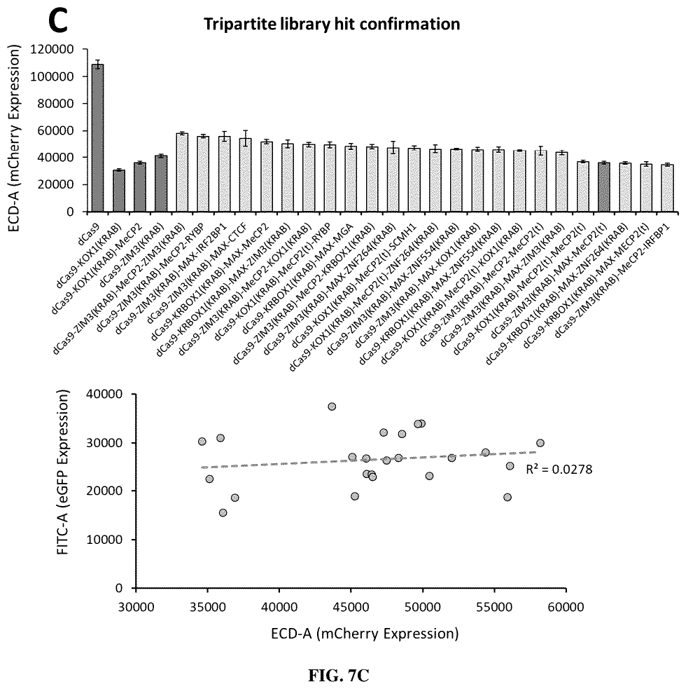

Next, it was evaluated if attaching multiple repressor domains to dCas9 synergistically improves gene knockdown. A library of bipartite repressors was generated by combining three KRAB domains (the newly described KRBOX1(KRAB), the best-in-class ZIM3(KRAB), and the historically utilized KOX1(KRAB)) with both KRAB and non-KRAB domains from initial experiments (FIG. 1B). Many bipartite repressor fusions exhibit improved capability over current gold standards, significantly outperforming dCas9-KOX1(KRAB)-MeCP2 and dCas9-ZIM3(KRAB) (FIG. 1C). Four unique repressor combinations (dCas9-KRBOX1(KRAB)-MAX, dCas9-ZIM3(KRAB)-MAX, dCas9-ZIM3(KRAB)-MeCP2, and dCas9-KOX1(KRAB)-MeCP2(t)) achieved significantly improved knockdown (˜20-30% better) compared to ZIM3(KRAB), the top performing CRISPRi system to date (FIG. 1D and FIG. 6). Performance of these novel fusions was not a function of endogenous expression levels (FIG. 7), and interestingly, no dual-KRAB domain fusions exhibited improved activity over repressors employing one KRAB domain (FIG. 1D), highlighting the importance of being able to recruit secondary co-factors to further gene knockdown.

Encouraged by these results, next a library of tripartite repressors was generated to determine if adding a third domain could further enhance performance of the most potent variants. A combinatorial library was designed fusing each of the four top-performing bipartite repressors (dCas9-KRBOX1(KRAB)-MAX, dCas9-ZIM3(KRAB)-MAX, dCas9-ZIM3(KRAB)-MeCP2, and dCas9-KOX1(KRAB)-MeCP2(t)) with both KRAB and non-KRAB domains (FIG. 1E). One novel tripartite fusions (dCas9-ZIM3(KRAB)-MAX-MeCP2(t)) exhibited superior knockdown efficiency compared to all four of the high-efficacy bipartite variants (FIG. 1F). The tripartite repressor fusions suffered from lower expression levels in HEK293T cells than bipartite or single dCas9-repressor combinations, limiting their ability to silence target gene expression (FIG. 7). In addition, individual members of tripartite fusions could have recruited identical co-factors or experienced misfolding or steric blockage of key domains for mediating protein-protein interactions.

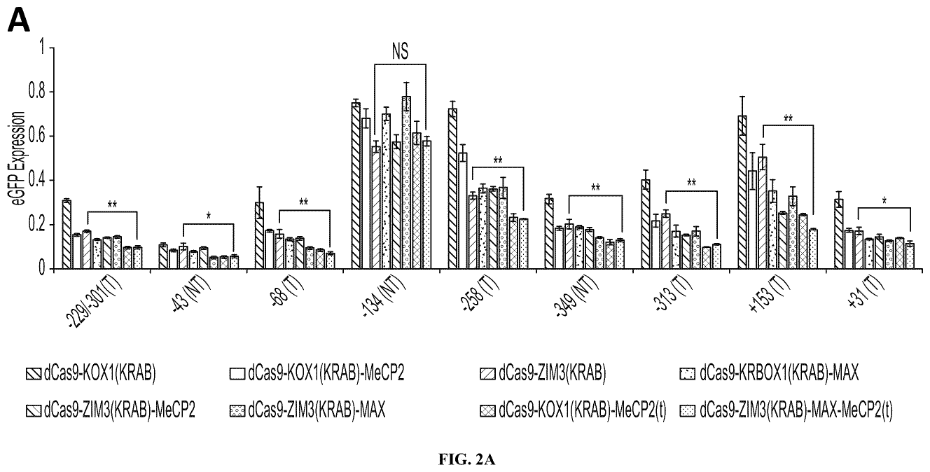

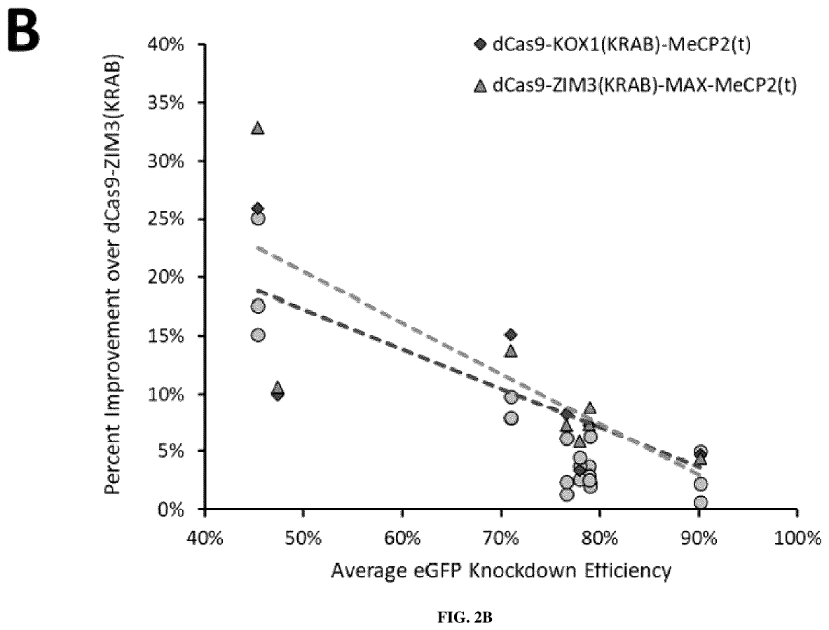

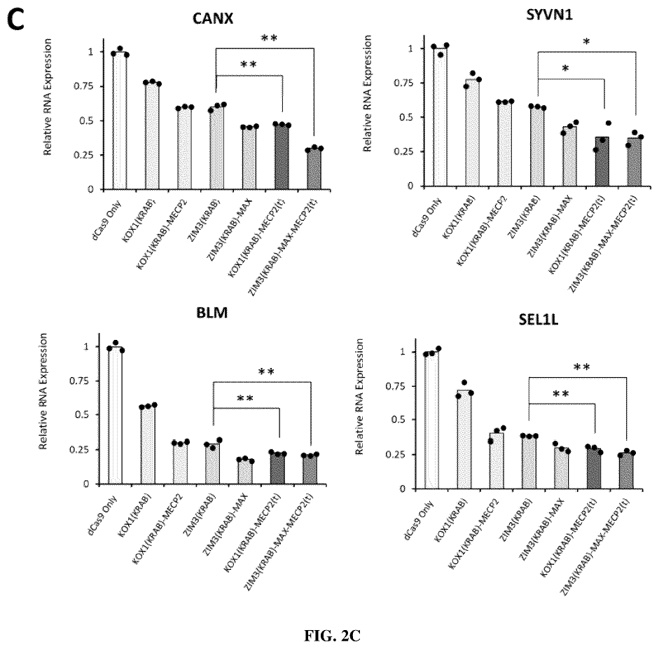

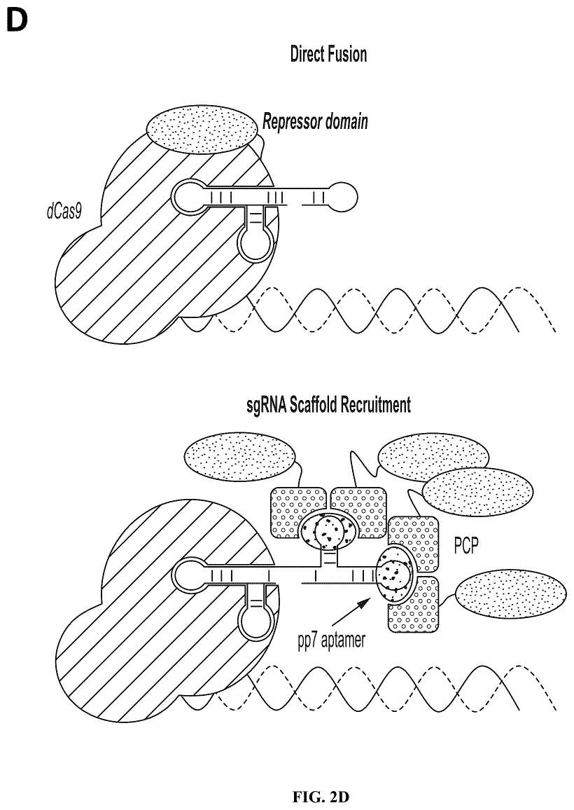

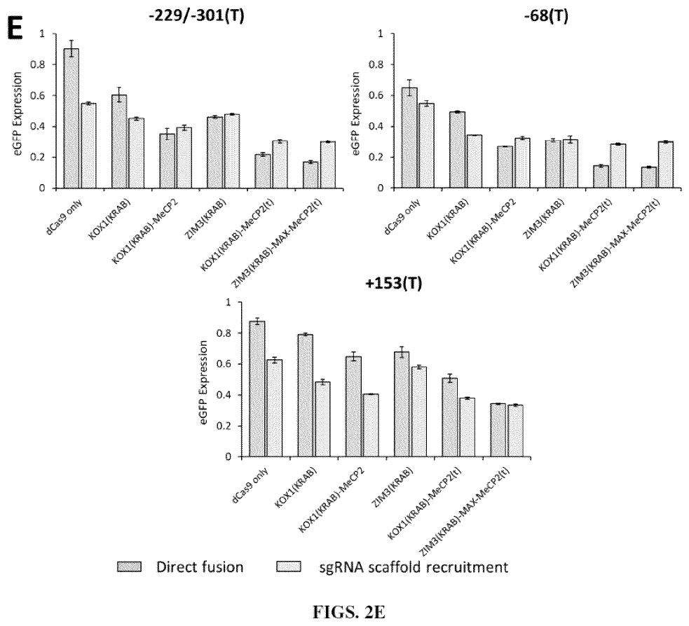

Evaluating CRISPRi Efficacy Across Different Genetic Loci and Targeting Modalities. Despite recent advances in rational sgRNA design and activity prediction, a significant challenge in applying CRISPRi systems in mammalian cells is that their performance is significantly impacted by the selected sgRNA. To determine whether engineered variants reduce the stochasticity arising from these criteria, a panel of sgRNAs were constructed targeting a SV40 promoter—eGFP reporter protein construct on template and non-template strands, both upstream and downstream of the transcription start site (TSS), and used it to compare the eGFP protein knockdown mediated by novel repressors and prior domains. Promisingly, dCas9-ZIM3(KRAB)-MAX-MeCP2 showed significantly improved repression compared to dCas9-ZIM3(KRAB) for 8 out of 9 sgRNAs of the panel (FIG. 2A). The potent gene repression was also independent of both target position and DNA strand, indicating that variants' high activity was not merely an artifact of the dual-targeting reporter sgRNA. Furthermore, repressors demonstrated greater improvement in gene repression when utilizing sgRNAs that had poorer functionality for previously described CRISPRi effectors, showing that the optimized dCas9-repressor fusions generated herein partially mitigate individual sgRNA limitations (FIG. 2B).

Building on these initial studies employing a synthetic reporter, it was next sought to confirm that the top-performing dCas9-repressor fusions outperformed current CRISPRi effectors in silencing endogenous genes, as there is also known stochasticity in performance of CRISPRi effectors in a gene by gene manner. The three top-performing novel variants, dCas9-ZIM3(KRAB)-MAX, dCas9-KOX1(KRAB)-MeCP2(t), and dCas9-ZIM3(KRAB)-MAX-MeCP2(t) were co-transfected into HEK293T cells with sgRNAs targeting one of four endogenous genes and then quantified gene knockdown using quantitative PCR with reverse transcription (RT-qPCR) in successfully transduced cells (sgRNA⁺/dCas9-repressor⁺). The dCas9-KOX1(KRAB)-MeCP2(t) and dCas9-ZIM3(KRAB)-MAX-MeCP2(t) effectors induced the strongest gene knockdown across all four loci tested (FIG. 2C) and also reduced gene-gene heterogeneity in CRISPRi activity compared to the dCas9-ZIM3(KRAB) gold standard (e.g., between 40 and 75% knockdown for ZIM3(KRAB) versus between 65% and 80% knockdown for ZIM3(KRAB)-MAX-MeCP2(t)). Some residual variation in degree of gene knockdown likely originates from individual sgRNA binding dynamics or local chromatin architecture. Importantly, all analyses were performed in comparison to a dCas9-only control to help differentiate between repressor-mediated expression knockdown or steric-blockade from dCas9-DNA binding.

CRISPRi systems typically employ direct fusion of repressor proteins to dCas9 though, alternative complexation strategies can be employed. To determine if novel repressor protein fusions are still effective to mediate gene knockdown in a scaffold-based effector recruitment system, the top-performing repressors were genetically fused to PP7 capsid protein (PCP) and sgRNAs encoding PP7 aptamers were utilized. This targeting approach was further compared with direct dCas9 fusions. Promisingly, top repressors recruited via PCP-PP7 aptamer binding still outperformed prior best in class effectors, although they were generally less effective than their corresponding dCas9 fusions, particularly for KOX1(KRAB)-MeCP2(t) and ZIM3(KRAB)-MAX-MeCP2(t) (FIG. 2D). Direct fusions and scaffold recruitment performed comparably when dCas9 was targeted to be downstream of the eGFP gene's TSS. This result likely relates to the PCP-PP7 system (which recruits four PCP-repressor domains simultaneously) sterically inhibits RNA polymerase progression more than dCas9 fusions alone.

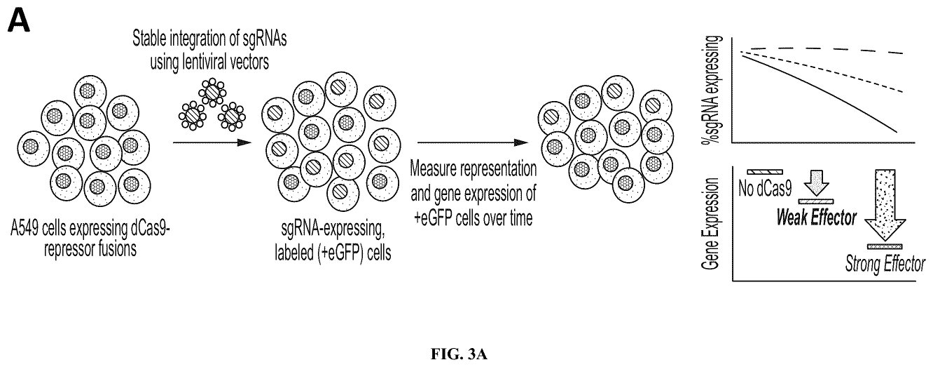

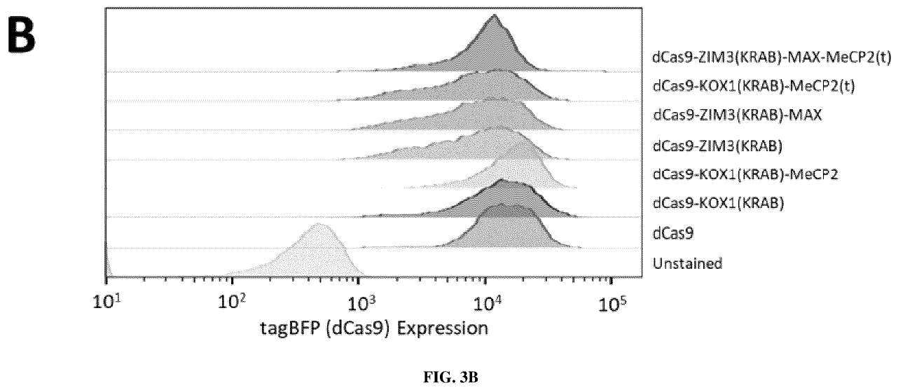

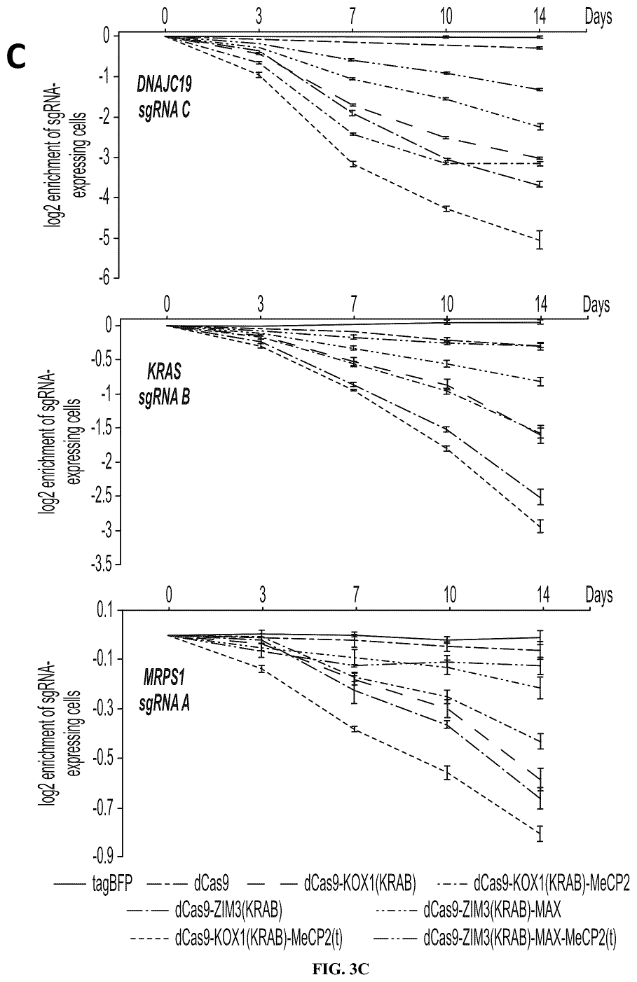

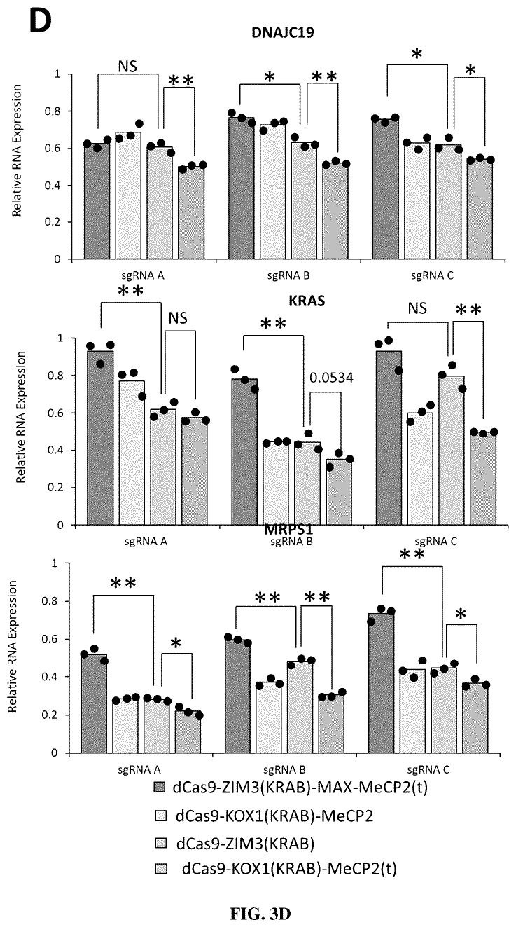

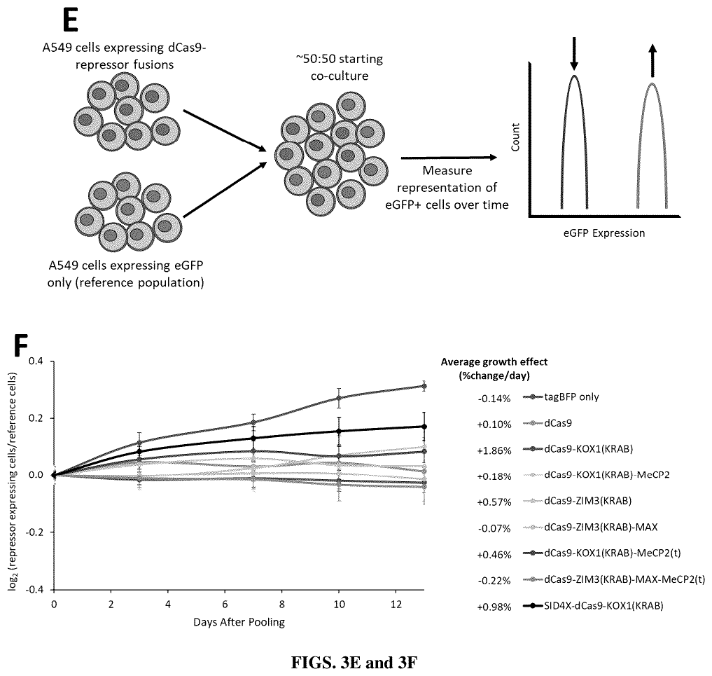

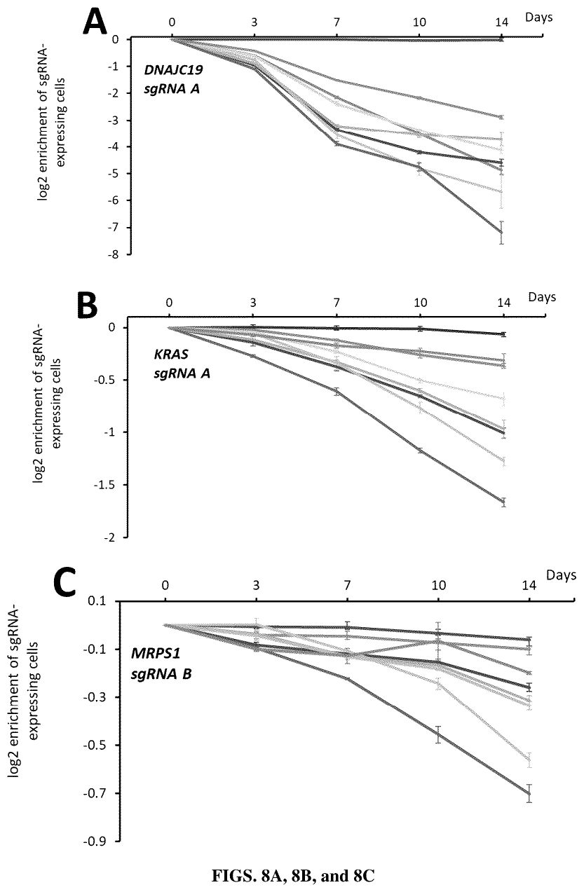

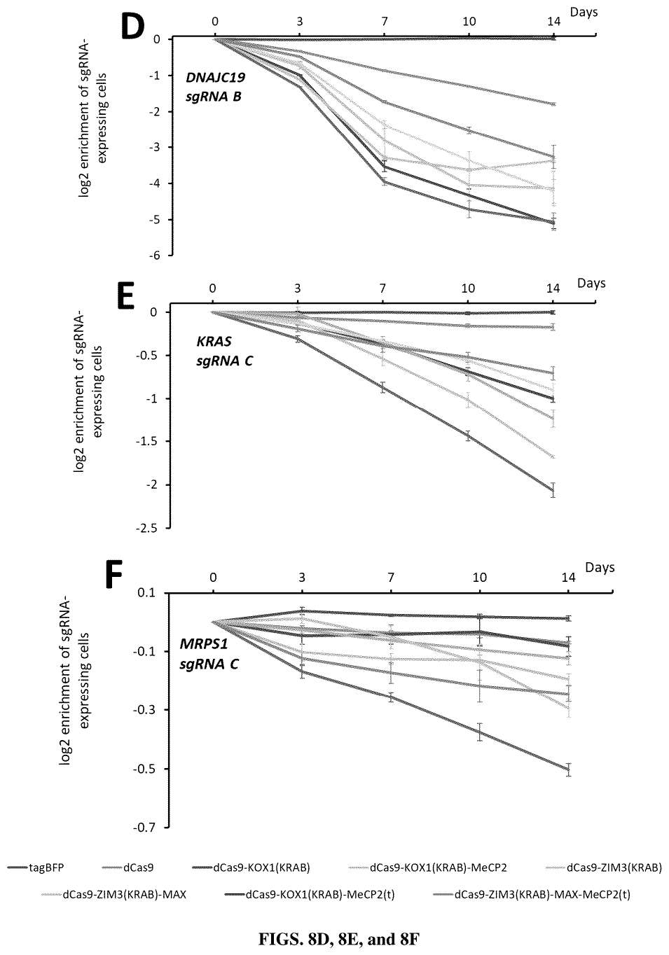

KOX1-MeCP2(t) Outperforms Existing Tools for Quantifying Gene Essentiality. Next, it was sought to evaluate the efficacy of novel repressor fusions by targeting essential genes, genes required for sustained cell growth and survival, and quantifying phenotype and gene expression changes. A549 cells were generated to constitutively express dCas9-repressor fusions and used competitive growth assays to measure proliferation rates. Each dCas9-repressor-expressing A549 cell line was tranduced with lentiviral cassettes bearing a single sgRNA and simultaneously expressed puromycin resistance and eGFP fluorescent tag to readily identify sgRNA-expressing cells. In separate experiments, three different genes were targeted using three sgRNAs each: (i) mitochondrial co-chaperone DNAJC19 (highly essential), (ii) GTPase and oncogene KRAS (moderately essential), and (iii) small ribosomal subunit protein MRPS11 (marginally essential). Post-transduction, the representation of eGFP-positive cells was monitered, presuming that cells bearing higher-activity repressor domains saw accelerated depletion of sgRNA (eGFP) expressing cells (FIG. 3A). A549 cells expressing dCas9-KOX1(KRAB)-MeCP2(t) experienced the highest levels of depletion across all tested sgRNAs, highlighting its improved repressive capability over current gold-standards dCas9-ZIM3(KRAB) and dCas9-KOX1(KRAB)-MeCP2 (FIGS. 3B, 3C, and 8). To further link phenotypic effects from CRISPRi perturbation to individual gene knockdown efficiencies, RNA expression levels were quantified for all three essential target genes using RT-qPCR. dCas9-KOX1(KRAB)-MeCP2(t) showed the strongest repression for 7 out of 9 targeting sgRNAs, correlating well with proliferation rates measured in A549 cells (FIG. 3D).

Because individual repressor domains fused to dCas9 for CRISPRi-mediated gene silencing are transcription factors recruiting factors co-regulating global transcriptional programs, their overexpression introduces undesired, non-specific effects on cellular function. To determine if novel dCas9-repressor variants impacted cellular proliferation, relative growth rates of cell lines was measured with integrated and constitutively expressed variants using a normalized co-culture assay (FIG. 3E). The dCas9-repressor-expressing A549 cell lines was seeded in a ˜1:1 ratio with A549 cells engineered with eGFP only (A549-eGFP), and then used flow cytometry to measure the representation of eGFP-positive cells over time. An A549-eGFP cells as an internal control was used to normalize the impact of cellular engineering and subsequent selection, and to aid in assessing effects of silencing on dropout rates of effector-expressing cells. Cell lines harboring the majority of repressor variants proliferated at the same rate as A549-eGFP cells, indicating that long-term expression of these fusions induces insignificant toxicity (FIG. 3F). Interestingly, stable integration of dCas9-KOX1(KRAB) (˜1.7% per day) and SID4x-dCas9-KOX1(KRAB) (˜1% per day) exhibited slight increases in growth compared to A549-eGFP control cells. This is contrary to previous work in K562 cells, which reported negligible for dCas9-KOX1(KRAB) and significant proliferation losses (˜6% per day) for SID4x-dCas9-KOX1(KRAB). These results showed that the engineered variants produce insignificant cellular toxicity from long-term overexpression (at least in A549 cells), presenting minimal risk to confound phenotype screens.

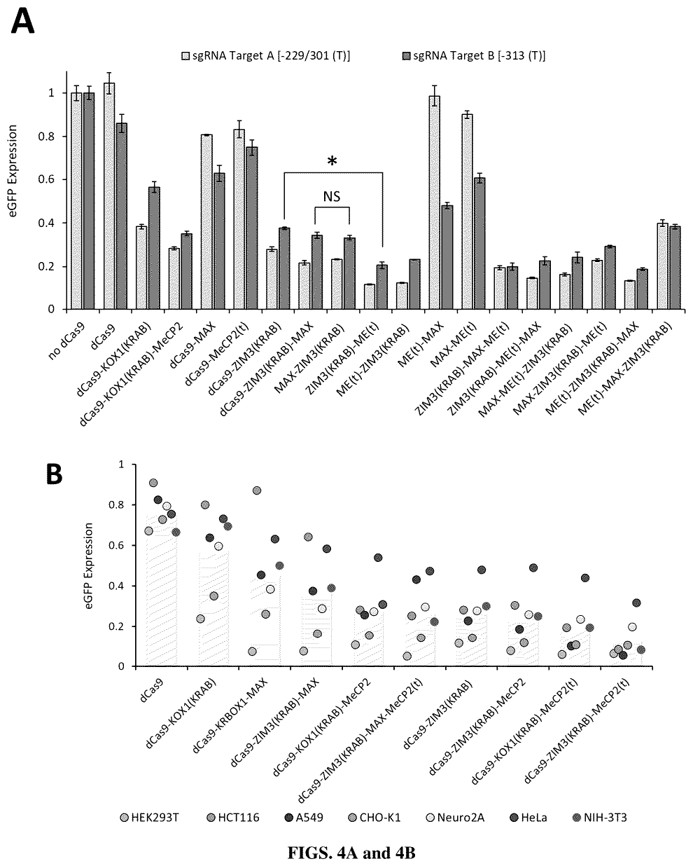

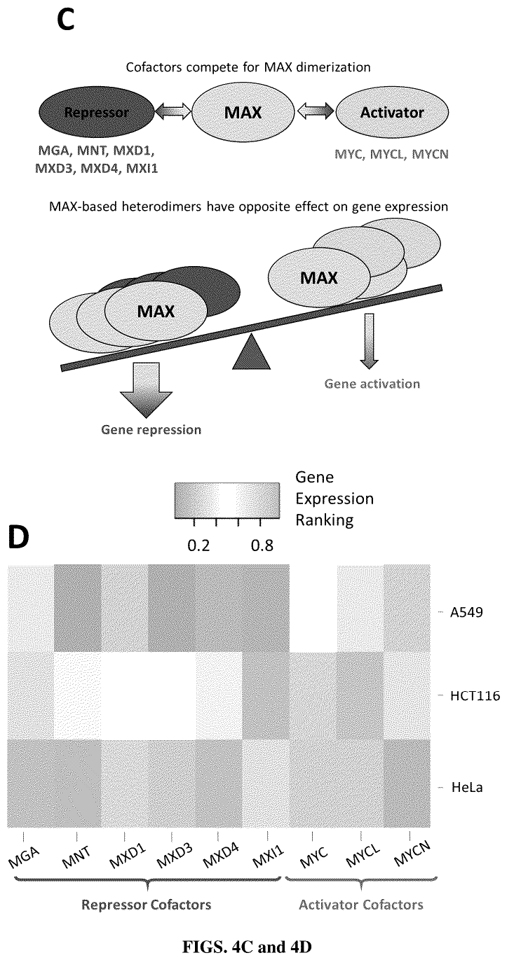

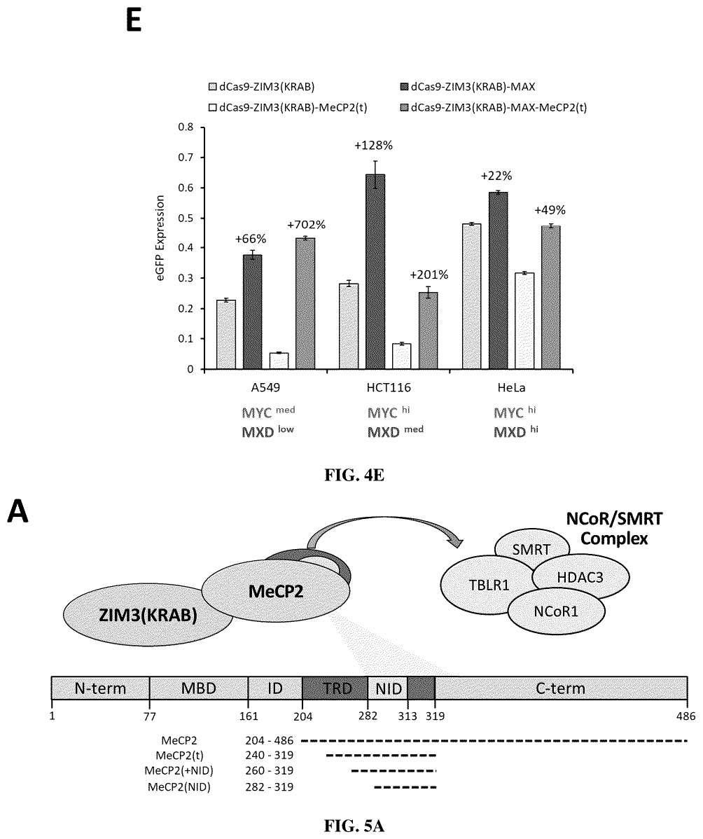

Investigating Domain Order Allows Isolation of Promising Repressor. To evaluate the impact of repressor order in dCas9-based fusions, which could cause misfolding or interference of proper effector recruitment, constructs were designed by fusing dCas9 to all possible combinatorial fusions of ZIM3(KRAB), MAX, and MeCP2(t) and assayed their gene silencing activity in HEK293T cells using two distinct sgRNA chaperons (FIG. 4A). Contrary to previous work characterizing multi-component transcriptional activator fusions, negligible changes in CRISPRi activity were observed when altering the order of both bipartite and tripartite fusion (FIG. 4A). Interestingly, the fusion dCas9-ZIM3(KRAB)-MeCP2(t) exhibited significantly improved knockdown compared to dCas9-ZIM3(KRAB), this new variant was continued to be used for additional studies.

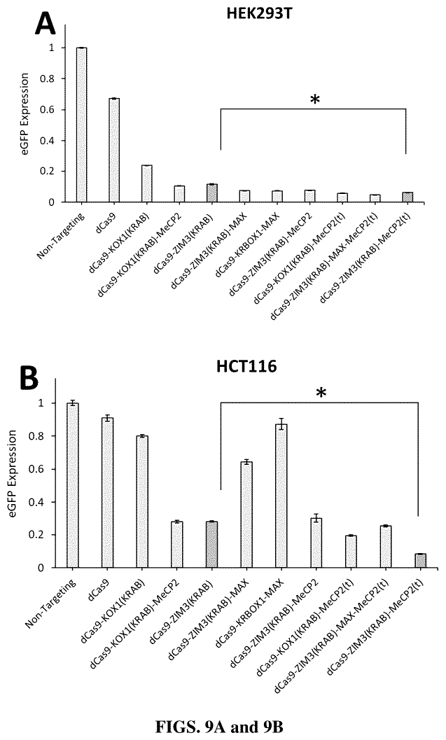

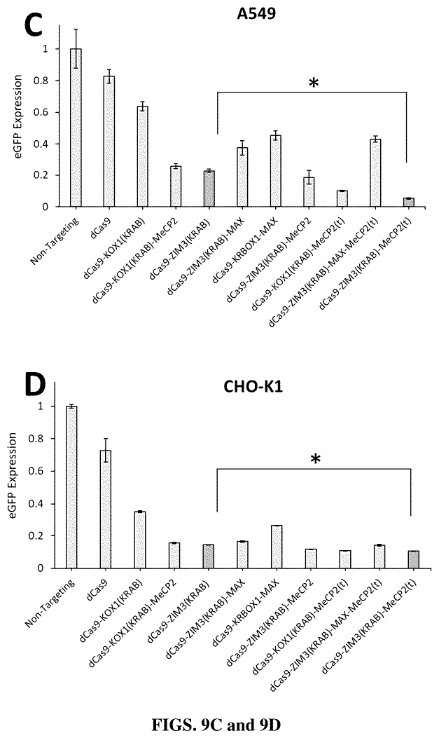

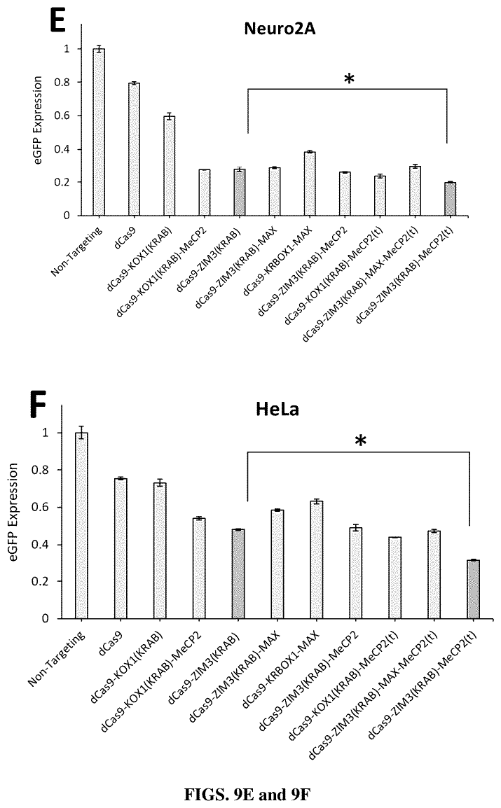

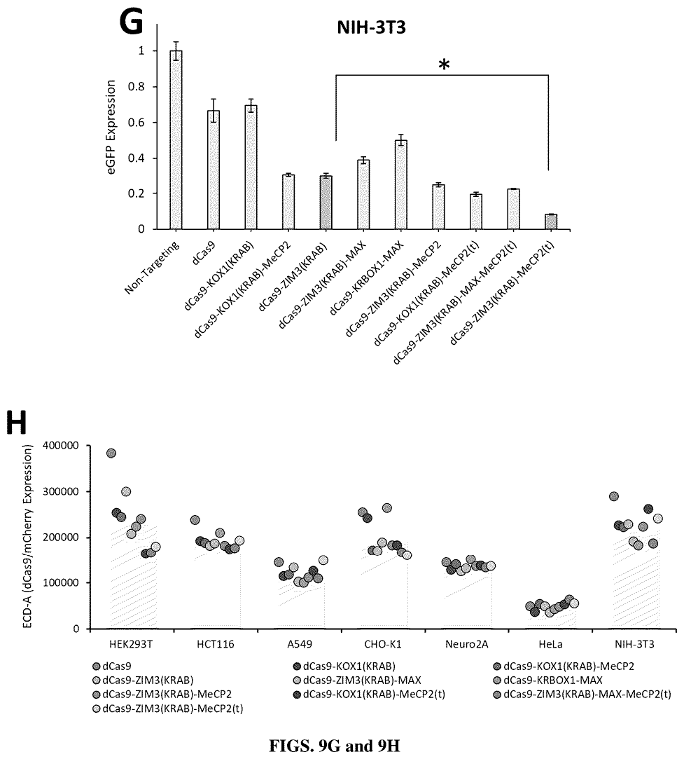

Novel Repressor Fusions Demonstrate Robust Activity Across Cell Lines. To further delve into cell-line dependent performance of repressor variants, a side-by-side comparisons of published gold-standard repressors and the novel fusions was conducted in 7 diverse mammalian cell lines: A549 (human lung adenocarcinoma), CHO-K1 (Chinese hamster ovary), HCT116 (human colon carcinoma), HEK293T (human embryonic kidney), HeLa (human cervical carcinoma), Neuro2A (mouse neuroblasts), and NIH-3T3 (mouse embryonic fibroblast). Using reporter co-transfection assays, dCas9-ZIM3(KRAB)-MeCP2(t) exhibited the strongest gene silencing across all cell lines tested (FIGS. 4B and 9A). The two most potent repressors, dCas9-ZIM3(KRAB)-MeCP2(t) and dCas9-KOX1(KRAB)-MeCP2(t), generated herein, outperformed dCas9-ZIM3(KRAB), the current CRISPRi gold standard, by an average of 50% and 31%, respectively, across cell lines (FIG. 4B), demonstrating their robust ability to mediate gene knockdown regardless of cell background.

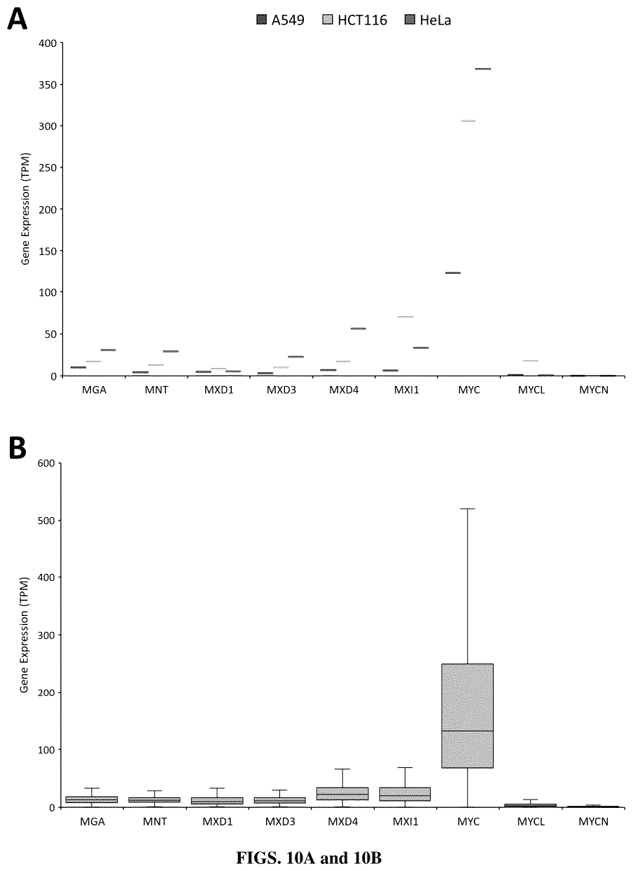

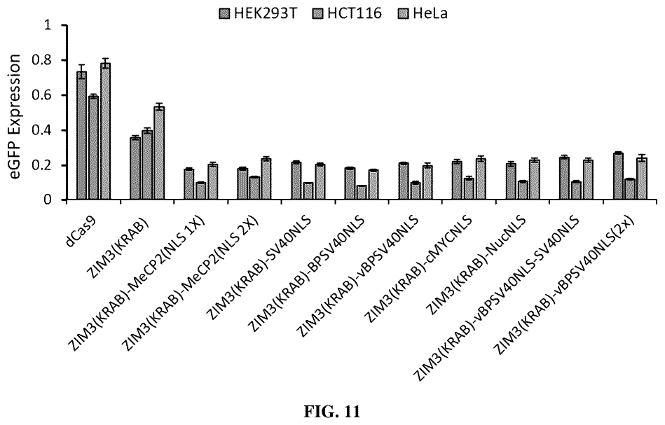

Co-Factor-Mediated Cell-Specific Performance Determinants. It was observed that the novel repressors variants that contained a MAX domain, which mediated gene knockdown exceptionally well in HEK293T cells, functioned markedly worse in other cell lines (FIGS. 3E and 9A). For instance, extremely potent activity of dCas9-ZIM3(KRAB)-MeCP2(t) was seen across cell lines that was markedly reduced in the dCas9-ZIM3(KRAB)-MAX-MeCP2(t) variant. Analysis of dCas9-repressor expression levels shows that this discrepancy does not result from poor effector expression (FIG. 9B). Therefore, it was contemplated that the MAX domain exhibits context-dependent transcriptional regulatory activity, dependent on its recruited co-factors, which has significant cell-line variability in their expression. There are nine high-confidence co-factors that dimerize with MAX, of which three facilitate transcriptional activation (MYC, MYCL, MYCN) and six initiate repression (MGA, MNT, MXD1, MXI1, MXD3, MXD4) (FIG. 4C). Using transcriptomics datasets, levels of the MAX-binding co-factors were quantified in A549, HCT116, and HeLa cells, three lines in which MAX harboring fusions performed significantly worse than their ‘non-MAX’ counterparts (FIGS. 4D and 10). HeLa cells had particularly high expression of MYC, which is known to be a particularly potent activator, and while A549 and HCT116 cells had lower MYC expression, they also had much lower relative expression of repression-mediating co-factors, when normalized by MYC expression level. Therefore, it is possible that either high MYC expression or a low ratio of repressing to activating cofactors reduce the gene knockdown activity of MAX-containing effectors on a cell-line dependent basis.

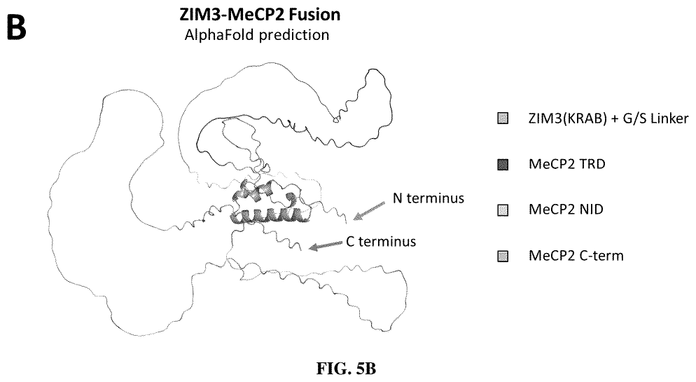

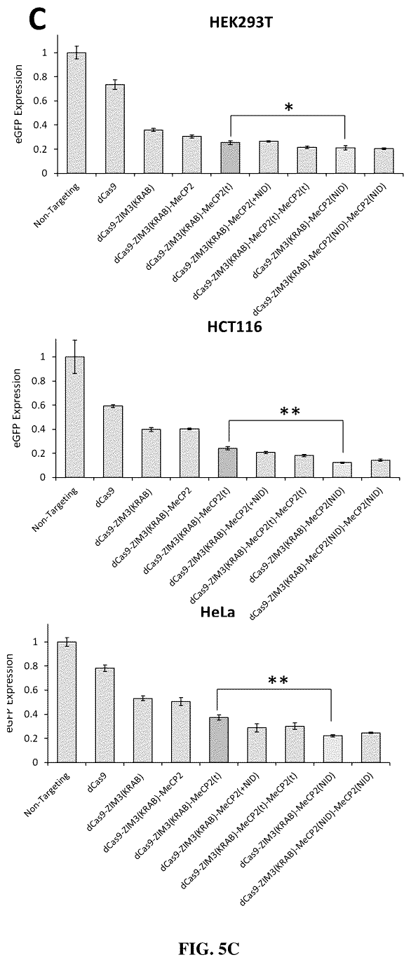

Further Truncating MeCP2 Leads to Improved Synergy with KRAB Domains. In comparisons of CRISPRi systems across mammalian cell lines, it was consistently observed that KRAB domain-based repressor fusions with MeCP2(t) significantly outperformed fusions employing the canonical MeCP2 transcriptional repressor domain, hereafter named MeCP2(full) for clarity (FIGS. 4B and 5A). MeCP2 mediates transcriptional silencing by recruiting both nuclear receptor corepressor (NCoR) and silencing mediator of retinoic acid and thyroid receptors (SMRT) through a ˜30AA motif known as the NcoR/SMRT interaction domain (NID). Because both MeCP2(t) and MeCP2(full) domains fully include this motif, it was contemplated as to what mechanistic factors underpin these two domains' discrepant performance.

An AlphaFold 2.0⁵⁶ protein structure predictions of three dCas9-repressor fusion proteins: ZIM3(KRAB), ZIM3(KRAB)-MeCP2(TRD), ZIM3(KRAB)-MeCP2(t) (FIG. 5B), was generated. These predictions revealed that MeCP2(TRD) and MeCP2(t) contain highly disordered C-terminal structure, which is common for transcription factors, but contributes to reduced accessibility of the NID domain. Therefore, a new bipartite fusion partnering ZIM3(KRAB) with only the MeCP2 NID domain (dCas9-ZIM3(KRAB)-MeCP2(NID)) was designed and it was compared with other ZIM3(KRAB)-based CRISPRi effectors using eGFP reporter assays in HEK293T, HeLa, and HCT116 cell lines. The dCas9-ZIM3(KRAB)-MeCP2(NID) significantly outperformed even dCas9-ZIM3(KRAB)-MeCP2(t) in all three cell lines (FIG. 5C). Compared to dCas9-ZIM3(KRAB) or dCas9-ZIM3(KRAB)-MeCP2, dCas9-ZIM3(KRAB)-MeCP2(NID) improved knockdown levels by more than two-fold in HCT116 and Hela cells, allowing for a reduction in gene expression approaching 80%. The ZIM3(KRAB) prior best-in-class variant enabled 50 to 60% knockdown. Therefore, this novel bipartite repressor confers best-in-class repression efficiency, both with smaller size (enabling delivery with package-limited viral vectors) and generalizable performance across cell lines.

### DISCUSSION

The superior performance of bipartite and tripartite variants, both reported here and elsewhere for CRISPR-dCas9 transcriptional activators, results from combining distinct, yet complimentary, mechanisms for modifying local epigenetic signatures. However, the understanding of how these individual repressor domains functionally work together to silence gene expression remains ambiguous. KRAB domains, known for their near-ubiquitous strong repressive activity, are almost exclusively implemented in CRISPRi applications in mammalian cells. KRAB domains effectively modulate transcription by interacting with TRIM28/KAP1. Although the screening efforts revealed that KRAB domains' have relatively strong gene knockdown efficiency that can be enhanced through addition of MeCP2 or MAX in HEK293T, no other domains conferred additional benefit when combined with KRAB-domains. These results show that KRAB-induced gene silencing may have few accessible synergistic mechanisms that can augment their function. Still, the present disclosure provides significant improvement to initial best-in-class repressor fusion by performing a relatively simple, sequence/motif guided truncation analysis. Specifically, it was shown that an initial and a secondary truncation of the canonical MeCP2 domain, yield MeCP2(t) and MeCP2(NID), respectively, in combination with the ZIM3(KRAB) domain bestowed excellent gene knockdown across cell lines. The truncations show that the superior potency of dCas9-ZIM3(KRAB)-MeCP2(NID) originates from improved accessibility of the NID motif to MeCP2's NCoR/SMRT cofactors or accessibility for TRIM28/HP1α recruitment by ZIM3(KRAB) by eliminating unnecessary coding regions.

Recent efforts chronicling repressor domains provide valuable resources for discovering entirely new CRISPRi systems with diverse mechanisms of action, modes of temporal control, and activity levels. The present disclosure identifies a small panel of diverse CRISPRi-compatible, non-KRAB domains with comparable repression efficiency to MeCP2. To expand on these findings, it would be instructive to significantly expand the panel of effective dCas9-compatible, non-KRAB dCas9-repressor domains, build a comprehensive understanding of their individual activities and co-factor identities, and construct multi-domain libraries to permit discovery of additional high-activity repressor combinations. Incorporating knowledge of recruited co-factors, cooperative epigenetic modifications, and repressor-repressor affinities enables greater means of rational design, permitting development of CRISPRi effector panels with well-characterized, diverse gene silencing efficiency and kinetics. Such efforts can help overcome any technical limitations associated with an upper limit on performance optimization of KRAB-based CRISPRi systems.

Improved characterization of fused transcriptional effectors is important not only for building enhanced synthetic biology tools, but also understanding the functional role of natural transcription factors with multiple effector domains. Emerging work has begun exploring the context-dependent behavior of several transcription factors by quantifying their affinities for their recruited co-factors. The results comparing bipartite and tripartite variants containing the MAX-domain across cell lines highlights this combinatorial crosstalk and demonstrates the importance of considering cell-cell differences in co-factor expression levels when selecting CRISPRi repressors for a given application. Additional analyses correlating endogenous co-factor (and dCas9-repressor) expression levels across a broad panel of mammalian cells helps clarify mechanistic relationships driving gene knockdown performance and predict optimal CRISPRi repressors for a given cell line.

Together, this work presents several novel CRISPRi repressors with best-in-class gene silencing efficiency. After demonstrating how combinatorial fusion of repressor domains can enhance gene knockdown, it is illustrated how a rational reduction of fusion protein size can further enhance CRISPRi function. In particular, the dCas9-ZIM3(KRAB)-MeCP2(NID) repressor, in which the MeCP2(NID) domain has been reduced in amino acid length by more than seven-fold, displayed the highest level of gene knockdown in every cell line tested. The repressor variants disclosed herein can enhance the efficacy of large-scale genotype-phenotype screens and aid in development of robust cellular engineering tools to build fundamental understanding of multi-modal transcriptional regulation in mammalian cells.

### Materials and Methods

Cell Culture. HEK293T, NIH-3T3, Neuro2A, and HeLa cell lines were maintained in DMEM/High Glucose (Cytiva) supplemented with 10% FBS (Fisher Scientific) and 1% Penicillin-Streptomycin (Millipore-Sigma). HCT116 cells (ATCC, CCL-247) were cultured in McCoy's 5A Modified Medium (Gibco) with 10% FBS and 1% Penicillin-Streptomycin. CHO-K1 and A549 (both parental and CRISPRi repressor-expressing) cell lines were maintained in DMEM/F-12 supplemented with 10% FBS and 1% Penicillin-Streptomycin. All cell lines were cultivated in 5% CO₂ at 37° C. and verified negative for Mycoplasma contamination on a semi-annual basis (every ˜6 months) using the Universal Mycoplasma Detection Kit (ATCC).

Plasmid Construction for CRISPRi Repressors and sgRNAs. Individual repressor domains for this study were acquired by PCR-amplification from a single-strand cDNA library. In short, total RNA from 10⁷ HEK293T cells was first purified using TRIzol Reagent (Invitrogen) and reverse-transcribed using a SuperScript VILO cDNA Synthesis Kit (Invitrogen). Repressor domains were then PCR-amplified from this first-strand cDNA pool using KOD Hot Start Polymerase (Novagen) using 250 ng of cDNA product per reaction with cycling conditions in-line with the manufacturer's protocol.

CRISPRi dCas9-repressor fusion plasmids for transient expression were constructed by inserting individual repressor domains into a custom Golden Gate compatible base vector (pEF1α-dCas9-mCherry) derived from the plasmid pSMART-sgRNA (Addgene #80427). This custom base vector, constructed via Gibson Assembly from the pSMART backbone digested with BamHI and XbaI (New England Biolabs), contains the EF1α promoter driving expression of human codon-optimized Streptococcus pyogenes dCas9 (with 1 N-terminal and 2 C-terminal SV40NLS elements), a C-terminal GS-rich linker with Esp3I restriction sites allowing insertion of various effector domains, and a P2A-mCherry marker enabling quantification of expression levels via flow cytometry. Golden Gate Assembly was employed for cloning single, bipartite, and tripartite dCas9-repressor fusions for analysis.

Constructs enabling stable integration of CRISPRi repressors were derived from an in-house custom base vector (pLV-dCas9-tagBFP). Briefly, this base vector uses a spleen focus-forming virus (SFFV) promoter with an upstream ubiquitous chromatin-opening element (UCOE) to drive expression of Streptococcus pyogenes dCas9, internal SV40NLS tags, a G/S-rich linker with Esp3I restriction sites to enable insertion of additional repressor domains, and a C-terminal tagBFP fluorescent marker linked via a T2A self-cleaving peptide. This plasmid was built by PCR-amplifying all requisite parts and inserting them with Gibson Assembly into lentiviral backbone pLeGO-C (Addgene #27348) linearized by digestion with XbaI and EcoRI (New England Biolabs). From this base vector, Golden Gate Assembly was used for building all lentiviral dCas9-repressor fusion constructs, and these vectors were transformed via electroporation into NEB Stable E. Coli (New England Biolabs) to prevent plasmid recombination during subsequent cloning steps.

The sgRNAs targeting either eGFP or individual endogenous genes for transient expression were cloned into a vector (pSMART-sgRNA-SV40-eGFP) constructed by adding the SV40-eGFP cassette via Gibson Assembly into the pSMART-sgRNA backbone. Following guide design, individual sgRNA constructs were made by annealing two complimentary oligonucleotides (Eurofins Genomics) containing the full sgRNA sequences and appropriate overhangs, then ligating the oligo product with pSMART-sgRNA-SV40-eGFP backbone pre-digested with Esp3I (New England Biolabs). Constructs for sgRNA integration were cloned using the identical ligation method into a custom lentiviral vector (pLV-sgRNA-EFlx-eGFP-T2A-PuroR) originally made by inserting PCR-amplified EFla, eGFP, and puromycin resistance marker within pXPR_050 (Addgene #96925) linearized by digestion with MluI and XmaI (New England Biolabs).

CRISPRi Reporter Knockdown ReporterAssays. For reporter assays, CRISPRi activity was quantified by co-transfecting two plasmids in HEK293T cells. Briefly, CRISPRi repressors encoded on mCherry-tagged plasmids (1) were mixed with a reporter plasmid (2) containing an sgRNA (GAAAGTCCCCAGGCTCCCCAGC (SEQ ID NO: 134)) recruiting the repressor to two sites on the proximal simian virus 40 (SV40) promoter regulating eGFP. HEK293T cells were initially seeded in either 24-well (150,000 cells/well, single repressor characterization) or 96-well plates (25,000 cells/well, all other experiments), then 24 hours later transfected with TransIT-LT1 (Mirus Bio) aligning with the manufacturer's protocol. Both eGFP and mCherry fluorescent markers were assayed 48 h later by using a Cytoflex S flow cytometer (Beckman Coulter). Analysis was excluded to cells expressing mCherry to control for variable transfection efficiency, and eGFP median intensity within this gated population was quantified for each group as a proxy for CRISPRi activity. Co-transfection experiments in other cell lines were completed using the same plasmids, assay design, and analysis technique. Respective lines were seeded within 24-well plates, and 24 h later transfected using TransIT-X2 Dynamic Delivery System (Mirus Bio) at the following DNA: reagent ratios as recommended by the supplier: A549(1:2), CHO-K1(1:2), HeLa(1:3), HCT116(1:2), Neuro2A(1:3), and NIH3T3(1:3).

Repressor Domain Library Screening. Library screening of bipartite and tripartite repressor fusion constructs was performed through reverse transfection of HEK293T cells. Following pooled Golden Gate assembly, libraries were transformed via standard electroporation into competent DH10β E. coli (New England Biolabs), subsequently plated, and single colonies were individually picked and purified (Qiagen). All isolated plasmids were normalized to 100 ng/μL to improve transfection efficiency uniformity in the screens. Transfections were next completed by adding 400 ng of each plasmid into individual wells of a tissue-culture treated 96-well plate (Falcon) and then adding a mixture of OptiMEM I Serum-Free Medium (18 uL/well) and TransIT-LT1 (1.2 ul/well) into each well as specified by the manufacturer's protocol. After a 20 min incubation at room temperature, HEK293T cells (50,000/well) were pipetted unto the assembled transfection complexes in each well. 48 h post-transfection, eGFP knockdown efficiency for each well was analyzed using the Cytoflex S flow cytometer. Preliminary screens utilized one biological replicate (one independent transfection), and before follow-up studies all hits were analyzed by Sanger sequencing to verify repressor identity and sequence fidelity.

Lentivirus Production. For large scale batches (dCas9-expressing constructs), lentivirus was produced in two 10-cm dishes by co-transfecting HEK293T cells with pMD2.G (Addgene #12259), psPAX2 (Addgene #12260), and transfer vector at a ratio of 1:4:5 (by mass). Transfections were performed using TransIT-LT1 (Mirus) adhering to the manufacturer's suggested protocol. 48 h after transfection, cell supernatants were collected, centrifuged at 1000×g for 5 min, filtered through 0.45 m syringe filters, precipitated with PEG-it™ viral precipitation solution (System Biosciences), and resuspended in ice-cold PBS before long-term storage at −80° C. For small scale batches (sgRNA cassettes), lentivirus was instead produced in 6-well plates using the same procedure and reagents, with quantities scaled down by cell surface area.

Transductions and Stable Cell Line Generation. A549 cell lines stably expressing various CRISPRi repressor domains or control fluorescent proteins were generated by first seeding parental A549 cells (60,000 cells/well) into 12-well plates, then transducing the cells at low MOI (˜0.2) in media containing 8 μg/mL polybrene (Millipore-Sigma). 24 h post-transduction, cells were thoroughly washed with PBS, then expanded for 3 days in T25 flasks. After recovery, CRISPRi repressor-expressing cells (marked by a BFP fluorescent marker) were sorted using BD FACS Melody into 96 well plates and expanded. All A549 repressor-expressing cell lines were validated through PCR-based analysis of each line's genomic DNA (gDNA) to confirm successful repressor integration. Briefly, gDNA was isolated from each cell line using a GeneJET Genomic DNA Purification Kit (Thermo Scientific), then viral transgenes were PCR amplified using KOD Hot Start Polymerase (Novagen) and subsequently analyzed by Sanger Sequencing to confirm repressor identity.

Cell Proliferation Assays. Internally controlled cellular growth assays were employed to evaluate the impact of stably expressing CRISPRi dCas9-repressor fusions in A549 cells. In short, this technique quantifies cell growth differences between cell populations of interest (expressing CRISPRi effectors linked to tagBFP via T2A driven by an SFFV promoter) and a reference cell population generated from the same parental line (expressing eGFP driven by an SFFV promoter). After producing all requisite A549 cells lines, cells from each repressor-expressing line were mixed with reference eGFP-expressing cells at a 1:1 ratio within 96 well plates. Immediately following mixing and periodically for 13 days, all cell line co-cultures were analyzed using the Cytoflex S to compute the ratio of eGFP⁺ cells to tagBFP⁺ cells to quantify growth effects over time.

A similar method was used to measure the effects of individual essential gene-targeting sgRNAs on cellular growth in A549 cells. Here, cell proliferation differences were quantified by stably integrating sgRNA constructs tagged with fluorescent markers (eGFP and puromycin resistance linked by a T2A, driven by an EF1α promoter) and evaluating the ratio of eGFP⁺ to eGFP⁻ cells over time. Strong and intermediate-efficiency sgRNA sequences were selected based on their relative efficacy from published whole-genome CRISPRi screens. Repressor-expressing A549 cell lines were first transduced with each sgRNA cassette (3 transductions per group) in 96-well plates with a lentiviral dose required to successfully integrate sgRNAs in ˜50% of host cells. 48 h after transduction, and every 2-3 days thereafter, all plates were sub-cultivated into new 96-well plates, and remaining cells analyzed by flow cytometry on the Cytoflex S to measure the representation of eGFP⁺ cells within each well.

RT-qPCR Gene Expression Analysis. Cell populations expressing both CRISPRi repressors and sgRNAs were selected with different methods to minimize variances in transfection/transduction efficiency prior to RNA extraction. For endogenous gene targeting in HEK293T cells, 500,000 cells/well were seeded into 6-well plates, and the following day co-transfected with a 1:2 (by mass) mixture of plasmids encoding sgRNAs (and eGFP) and dCas9-repressor fusions (marked with P2A-mCherry markers). All sgRNAs were selected to recognize −100 to +200 bp proximal to each target gene's TSS. 48 h post-transfection, 50,000 eGFP⁺/mCherry⁺ double-positive cells were sorted using BD FACS Melody, seeded into 48-well plates, and recovered for 24 h prior to RNA collection. For A549 cell lines stably expressing CRISPRi repressors, cells were seeded at 20,000 cells/well into 48-well plates in biological triplicates, and 24 h later transduced at low MOI (˜0.5) using the same sgRNA-expressing lentivirus (marked by eGFP and puromycin resistance connected by a T2A self-cleaving peptide) used in the cell proliferation assays. Transduced cells were recovered in fresh culture media at 24 h post-transduction, and the next day were re-seeded with 3 μg/ml puromycin and grown for 4d to select for sgRNA-expressing cells leading up to RNA extraction.

Total RNA was extracted and stored using TRIzol Reagent (Invitrogen) and subsequently purified using RNeasy Micro Kits (Qiagen). To quantify mRNA abundances, reactions containing 50 ng total RNA were set up with Universal One-Step RT-qPCR Kit (New England Biolabs) in 96-well plates. All plates were analyzed on a StepOnePlus Real-Time PCR System (Applied Biosystems) with the following cycling conditions: 55° C. for 10 min, 95° C. for 1 min, 40 cycles of 95° C. for 10 s then 60° C. for 60 s (+plate read), then a final 60° C.-95° C. melt curve. RNA relative abundances, normalized to the housekeeping gene IPO8⁶⁷, were then computed using the 2-ΔΔCₜmethod. Primers were designed to span exon-exon junctions of each gene target.

Software. FlowJo (version 9) was used to process and analyze data acquired from all flow cytometry experiments.

Statistics and Reproducibility. For studies evaluating CRISPRi-mediated knockdown of either reporter or endogenous genes, at least three independent biological replicates (separate transfections) per condition were used. All replicate counts, and statistical tests to identify significance are indicated in each figure caption within the manuscript.

### Example 2: dCas-Repressor Fusions with Nuclear Localization Signals (NLS)

To investigate the impact of nuclear localization on dCas9-repressor performance, various NLS elements were fused to the C-terminus of dCas9-ZIM3(KRAB) and the knockdown efficiency of each construct was tested using a SV40-eGFP reporter assay in HEK293T, HCT116, and HeLa cell lines (FIG. 11). It was determined that all NLS additions significantly enhanced gene knockdown, resulting in an average improvement of ˜50%, regardless of cell type. These results indicate current gold standard repressors can be improved through additional means besides utilizing different, or truncated, effector domains, and that nuclear localization are a significant variable influencing CRISPRi efficacy in mammalian systems.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES
1.
dCas9 DNA
SEQ ID NO: 1
ATGGACAAGAAGTATTCTATCGGACTGGCCATCGGGACTAATAGC
GTCGGGTGGGCCGTGATCACTGACGAGTACAAGGTGCCCTCTAAG
AAGTTCAAGGTGCTCGGGAACACCGACCGGCATTCCATCAAGAAA
AATCTGATCGGAGCTCTCCTCTTTGATTCAGGGGAGACCGCTGAA
GCAACCCGCCTCAAGCGGACTGCTAGACGGCGGTACACCAGGAGG
AAGAACCGGATTTGTTACCTTCAAGAGATATTCTCCAACGAAATG
GCAAAGGTCGACGACAGCTTCTTCCATAGGCTGGAAGAATCATTC
CTCGTGGAAGAGGATAAGAAGCATGAACGGCATCCCATCTTCGGT
AATATCGTCGACGAGGTGGCCTATCACGAGAAATACCCAACCATC
TACCATCTTCGCAAAAAGCTGGTGGACTCAACCGACAAGGCAGAC
CTCCGGCTTATCTACCTGGCCCTGGCCCACATGATCAAGTTCAGA
GGCCACTTCCTGATCGAGGGCGACCTCAATCCTGACAATAGCGAT
GTGGATAAACTGTTCATCCAGCTGGTGCAGACTTACAACCAGCTC
TTTGAAGAGAACCCCATCAATGCAAGCGGAGTCGATGCCAAGGCC
ATTCTGTCAGCCCGGCTGTCAAAGAGCCGCAGACTTGAGAATCTT
ATCGCTCAGCTGCCGGGTGAAAAGAAAAATGGACTGTTCGGGAAC
CTGATTGCTCTTTCACTTGGGCTGACTCCCAATTTCAAGTCTAAT
TTCGACCTGGCAGAGGATGCCAAGCTGCAACTGTCCAAGGACACC
TATGATGACGATCTCGACAACCTCCTGGCCCAGATCGGTGACCAA
TACGCCGACCTTTTCCTTGCTGCTAAGAATCTTTCTGACGCCATC
CTGCTGTCTGACATTCTCCGCGTGAACACTGAAATCACCAAGGCC
CCTCTTTCAGCTTCAATGATTAAGCGGTATGATGAGCACCACCAG
GACCTGACCCTGCTTAAGGCACTCGTCCGGCAGCAGCTTCCGGAG
AAGTACAAGGAAATCTTCTTTGACCAGTCAAAGAATGGATACGCC
GGCTACATCGACGGAGGTGCCTCCCAAGAGGAATTTTATAAGTTT
ATCAAACCTATCCTTGAGAAGATGGACGGCACCGAAGAGCTCCTC
GTGAAACTGAATCGGGAGGATCTGCTGCGGAAGCAGCGCACTTTC
GACAATGGGAGCATTCCCCACCAGATCCATCTTGGGGAGCTTCAC
GCCATCCTTCGGCGCCAAGAGGACTTCTACCCCTTTCTTAAGGAC
AACAGGGAGAAGATTGAGAAAATTCTCACTTTCCGCATCCCCTAC
TACGTGGGACCCCTCGCCAGAGGAAATAGCCGGTTTGCTTGGATG
ACCAGAAAGTCAGAAGAAACTATCACTCCCTGGAACTTCGAAGAG
GTGGTGGACAAGGGAGCCAGCGCTCAGTCATTCATCGAACGGATG
ACTAACTTCGATAAGAACCTCCCCAATGAGAAGGTCCTGCCGAAA
CATTCCCTGCTCTACGAGTACTTTACCGTGTACAACGAGCTGACC
AAGGTGAAATATGTCACCGAAGGGATGAGGAAGCCCGCATTCCTG
TCAGGCGAACAAAAGAAGGCAATTGTGGACCTTCTGTTCAAGACC
AATAGAAAGGTGACCGTGAAGCAGCTGAAGGAGGACTATTTCAAG
AAAATTGAATGCTTCGACTCTGTGGAGATTAGCGGGGTCGAAGAT
CGGTTCAACGCAAGCCTGGGTACCTACCATGATCTGCTTAAGATC
ATCAAGGACAAGGATTTTCTGGACAATGAGGAGAACGAGGACATC
CTTGAGGACATTGTCCTGACTCTCACTCTGTTCGAGGACCGGGAA
ATGATCGAGGAGAGGCTTAAGACCTACGCCCATCTGTTCGACGAT
AAAGTGATGAAGCAACTTAAACGGAGAAGATATACCGGATGGGGA
CGCCTTAGCCGCAAACTCATCAACGGAATCCGGGACAAACAGAGC
GGAAAGACCATTCTTGATTTCCTTAAGAGCGACGGATTCGCTAAT
CGCAACTTCATGCAACTTATCCATGATGATTCCCTGACCTTTAAG
GAGGACATCCAGAAGGCCCAAGTGTCTGGACAAGGTGACTCACTG
CACGAGCATATCGCAAATCTGGCTGGTTCACCCGCTATTAAGAAG
GGTATTCTCCAGACCGTGAAAGTCGTGGACGAGCTGGTCAAGGTG
ATGGGTCGCCATAAACCAGAGAACATTGTCATCGAGATGGCCAGG
GAAAACCAGACTACCCAGAAGGGACAGAAGAACAGCAGGGAGCGG
ATGAAAAGAATTGAGGAAGGGATTAAGGAGCTCGGGTCACAGATC
CTTAAAGAGCACCCGGTGGAAAACACCCAGCTTCAGAATGAGAAG
CTCTATCTGTACTACCTTCAAAATGGACGCGATATGTATGTGGAC
CAAGAGCTTGATATCAACAGGCTCTCAGACTACGACGTGGACGCC
ATCGTCCCTCAGAGCTTCCTCAAAGACGACTCAATTGACAATAAG
GTGCTGACTCGCTCAGACAAGAACCGGGGAAAGTCAGATAACGTG
CCCTCAGAGGAAGTCGTGAAAAAGATGAAGAACTATTGGCGCCAG
CTTCTGAACGCAAAGCTGATCACTCAGCGGAAGTTCGACAATCTC
ACTAAGGCTGAGAGGGGCGGACTGAGCGAACTGGACAAAGCAGGA
TTCATTAAACGGCAACTTGTGGAGACTCGGCAGATTACTAAACAT
GTCGCCCAAATCCTTGACTCACGCATGAATACCAAGTACGACGAA
AACGACAAACTTATCCGCGAGGTGAAGGTGATTACCCTGAAGTCC
AAGCTGGTCAGCGATTTCAGAAAGGACTTTCAATTCTACAAAGTG
CGGGAGATCAATAACTATCATCATGCTCATGACGCATATCTGAAT
GCCGTGGTGGGAACCGCCCTGATCAAGAAGTACCCAAAGCTGGAA
AGCGAGTTCGTGTACGGAGACTACAAGGTCTACGACGTGCGCAAG
ATGATTGCCAAATCTGAGCAGGAGATCGGAAAGGCCACCGCAAAG
TACTTCTTCTACAGCAACATCATGAATTTCTTCAAGACCGAAATC
ACCCTTGCAAACGGTGAGATCCGGAAGAGGCCGCTCATCGAGACT
AATGGGGAGACTGGCGAAATCGTGTGGGACAAGGGCAGAGATTTC
GCTACCGTGCGCAAAGTGCTTTCTATGCCTCAAGTGAACATCGTG
AAGAAAACCGAGGTGCAAACCGGAGGCTTTTCTAAGGAATCAATC
CTCCCCAAGCGCAACTCCGACAAGCTCATTGCAAGGAAGAAGGAT
TGGGACCCTAAGAAGTACGGCGGATTCGATTCACCAACTGTGGCT
TATTCTGTCCTGGTCGTGGCTAAGGTGGAAAAAGGAAAGTCTAAG
AAGCTCAAGAGCGTGAAGGAACTGCTGGGTATCACCATTATGGAG
CGCAGCTCCTTCGAGAAGAACCCAATTGACTTTCTCGAAGCCAAA
GGTTACAAGGAAGTCAAGAAGGACCTTATCATCAAGCTCCCAAAG
TATAGCCTGTTCGAACTGGAGAATGGGCGGAAGCGGATGCTCGCC
TCCGCTGGCGAACTTCAGAAGGGTAATGAGCTGGCTCTCCCCTCC
AAGTACGTGAATTTCCTCTACCTTGCAAGCCATTACGAGAAGCTG
AAGGGGAGCCCCGAGGACAACGAGCAAAAGCAACTGTTTGTGGAG
CAGCATAAGCATTATCTGGACGAGATCATTGAGCAGATTTCCGAG
TTTTCTAAACGCGTCATTCTCGCTGATGCCAACCTCGATAAAGTC
CTTAGCGCATACAATAAGCACAGAGACAAACCAATTCGGGAGCAG
GCTGAGAATATCATCCACCTGTTCACCCTCACCAATCTTGGTGCC
CCTGCCGCATTCAAGTACTTCGACACCACCATCGACCGGAAACGC
TATACCTCCACCAAAGAAGTGCTGGACGCCACCCTCATCCACCAG
AGCATCACCGGACTTTACGAAACTCGGATTGACCTCTCACAGCTC
GGAGGTGAT
2.
dCas9 protein
SEQ ID NO: 2
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDAIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD
3.
Linker between dCas9 and repressor domain DNA
SEQ ID NO: 3
GGCTCCGGAAGTGGGTCTAGAGGTGGAGCC
4.
Linker between dCas9 and repressor domain peptide
SEQ ID NO: 4
GSGSGSRGGA
5.
Linker between two or more repressor
domains DNA (in bipartite and
tripartite fusions)
SEQ ID NO: 5
GGCTCTGGCAGCGCTTCTGCTGGA
6.
Linker between two or more repressor
domains peptide (in bipartite and
tripartite fusions)
SEQ ID NO: 6
GSGSASAG
7.
Linker between two or more repressor
domains DNA (in tripartite fusions)
SEQ ID NO: 7
GGCAGCGCTTCTGCTGGA
8.
Linker between two or more repressor
domains peptide (in tripartite
fusions)
SEQ ID NO: 8
GSASAG
9.
KRBOX1 DNA
SEQ ID NO: 9
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
10.
KRBOX1 peptide
SEQ ID NO: 10
MTAVSLTTRPQESVAFEDVAVYFTTKEWAIMVPAERALYRDVMLE
NYEAVAFVVPPTSKPALVSHLEQGKESCFTQPQGVLSRNDWRAGW
11.
TRIM28 DNA
SEQ ID NO: 11
CACTGCGGCGTGTGCAGAGAGCGCCTGCGACCCGAGAGGGAGCCC
CGCCTGCTGCCCTGTTTGCACTCGGCCTGTAGTGCCTGCTTAGGG
CCCGCGGCCCCCGCCGCCGCCAACAGCTCGGGGGACGGCGGGGCG
GCGGGCGACGGCACCGTGGTGGACTGTCCCGTGTGCAAGCAACAG
TGCTTCTCCAAAGACATCGTGGAGAATTATTTCATGCGTGATAGT
GGCAGCAAGGCTGCCACCGACGCCCAGGATGCGAACCAGTGCTGC
ACTAGCTGTGAGGATAATGCCCCAGCCACCAGCTACTGTGTGGAG
TGCTCGGAGCCTCTGTGTGAGACCTGTGTAGAGGCGCACCAGCGG
GTGAAGTACACCAAGGACCATACTGTGCGCTCTACTGGGCCAGCC
AAGTCTCGGGATGGTGAACGTACTGTCTATTGCAACGTACACAAG
CATGAACCCCTTGTGCTGTTTTGTGAGAGCTGTGATACTCTCACC
TGCCGAGACTGCCAGCTCAATGCCCACAAGGACCACCAGTACCAG
TTCTTAGAGGATGCAGTGAGGAACCAGCGCAAGCTCCTGGCCTCA
CTGGTGAAGCGCCTTGGGGACAAACATGCAACATTGCAGAAGAGC
ACCAAGGAGGTTCGCAGCTCAATCCGCCAGGTGTCTGACGTACAG
AAGCGTGTGCAAGTGGATGTCAAGATGGCCATCCTGCAGATCATG
AAGGAGCTGAATAAGCGGGGCCGTGTGCTGGTCAATGATGCCCAG
AAGGTGACTGAGGGGCAGCAGGAGCGCCTGGAGCGGCAGCACTGG
ACCATGACCAAGATCCAGAAGCACCAGGAGCACATTCTGCGCTTT
GCCTCTTGGGCTCTGGAGAGTGACAACAACACAGCCCTTTTGCTT
TCTAAGAAGTTGATCTACTTCCAGCTGCACCGGGCCCTCAAGATG
12.
TRIM28 peptide
SEQ ID NO: 12
HCGVCRERLRPEREPRLLPCLHSACSACLGPAAPAAANSSGDGGA
AGDGTVVDCPVCKQQCFSKDIVENYFMRDSGSKAATDAQDANQCC
TSCEDNAPATSYCVECSEPLCETCVEAHQRVKYTKDHTVRSTGPA
KSRDGERTVYCNVHKHEPLVLFCESCDTLTCRDCQLNAHKDHQYQ
FLEDAVRNQRKLLASLVKRLGDKHATLQKSTKEVRSSIRQVSDVQ
KRVQVDVKMAILQIMKELNKRGRVLVNDAQKVTEGQQERLERQHW
TMTKIQKHQEHILRFASWALESDNNTALLLSKKLIYFQLHRALKM
13.
RYBP DNA
SEQ ID NO: 13
GATCCTCCTAGTGAAGCAAACAGCATACAGTCTGCAAATGCTACA
ACAAAGACCAGCGAAACAAATCACACCTCAAGGCCCCGGCTGAAA
AACGTGGACAGGAGCACTGCACAGCAGTTGGCAGTAACTGTGGGC
AACGTCACCGTCATTATCACAGACTTTAAGGAAAAGACTCGCTCC
TCATCGACATCCTCATCCACAGTGACCTCCAGTGCAGGGTCAGAA
CAGCAGAACCAGAGCAGCTCGGGGTCAGAGAGCACAGACAAGGGC
TCCTCCCGTTCCTCCACGCCAAAGGGCGACATG
14.
RYBP peptide
SEQ ID NO: 14
DPPSEANSIQSANATTKTSETNHTSRPRLKNVDRSTAQQLAVTVG
NVTVIITDFKEKTRSSSTSSSTVTSSAGSEQQNQSSSGSESTDKG
SSRSSTPKGDM
15.
CBX1 DNA
SEQ ID NO: 15
GATTCTGAAGATAAGGGAGAGGAGAGCAAACCAAAGAAGAAGAAA
GAAGAGTCAGAAAAGCCACGAGGCTTTGCTCGAGGTTTGGAGCCG
GAGCGGATTATTGGAGCTACAGACTCCAGTGGAGAGCTCATGTTC
CTGATGAAATGGAAAAACTCTGATGAGGCTGACCTGGTCCCTGCC
AAGGAAGCCAATGTCAAGTGCCCACAGGTTGTCATATCCTTCTAT
GAGGAAAGGCTGACGTGGCATTCCTACCCCTCGGAGGATGATGAC
AAAAAAGATGACAAGAAC
16.
CBX1 peptide
SEQ ID NO: 16
DSEDKGEESKPKKKKEESEKPRGFARGLEPERIIGATDSSGELMF
LMKWKNSDEADLVPAKEANVKCPQVVISFYEERLTWHSYPSEDDD
KKDDKN
17.
MeCP2(t) DNA
SEQ ID NO: 17
ACCACATCCACCCAGGTCATGGTGATCAAACGCCCCGGCAGGAAG
CGAAAAGCTGAGGCCGACCCTCAGGCCATTCCCAAGAAACGGGGC
CGAAAGCCGGGGAGTGTGGTGGCAGCCGCTGCCGCCGAGGCCAAA
AAGAAAGCCGTGAAGGAGTCTTCTATCCGATCTGTGCAGGAGACC
GTACTCCCCATCAAGAAGCGCAAGACCCGGGAGACCGTCAGCATC
GAGGTCAAGGAAGTG
18.
MeCP2(t) peptide
SEQ ID NO: 18
TTSTQVMVIKRPGRKRKAEADPQAIPKKRGRKPGSVVAAAAAEAK
KKAVKESSIRSVQETVLPIKKRKTRETVSIEVKEV
19.
SCMH1 DNA
SEQ ID NO: 19
TCCCCAGGGTCGGACCGATACCTGGAGAGCCGCGATGCCTCTCGA
CTGAGTGGCCGGGACCCCTCCTCATGGACAGTCGAGGATGTGATG
CAGTTTGTCCGGGAAGCTGATCCTCAGCTTGGACCCCACGCTGAC
CTGTTTCGCAAACACGAGATCGATGGCAAGGCCCTGCTGCTGCTG
CGCAGTGACATGATGATGAAGTACATGGGCCTGAAGCTGGGGCCT
GCACTCAAGCTCTCCTACCACATTGACCGGCTGAAGCAGGGCAAG
TTC
20.
SCMH1 peptide
SEQ ID NO: 20
SPGSDRYLESRDASRLSGRDPSSWTVEDVMQFVREADPQLGPHAD
LFRKHEIDGKALLLLRSDMMMKYMGLKLGPALKLSYHIDRLKQGK
F
21.
CTCF DNA
SEQ ID NO: 21
GTTGTAAATATGGAGGAACAGCCCATAAACATAGGAGAACTTCAG
CTTGTTCAAGTACCTGTTCCTGTGACTGTACCTGTTGCTACCACT
TCAGTAGAAGAACTTCAGGGGGCTTATGAAAATGAAGTGTCTAAA
GAGGGCCTTGCGGAAAGTGAACCCATGATATGCCACACCCTACCT
TTGCCTGAAGGGTTTCAGGTGGTTAAAGTGGGGGCCAATGGAGAG
GTGGAGACACTAGAACAAGGGGAACTTCCACCCCAGGAAGATCCT
AGTTGGCAAAAAGACCCAGACTATCAGCCACCAGCCAAAAAAACA
AAGAAAACCAAAAAGAGC
22.
CTCF peptide
SEQ ID NO: 22
VVNMEEQPINIGELQLVQVPVPVTVPVATTSVEELQGAYENEVSK
EGLAESEPMICHTLPLPEGFQVVKVGANGEVETLEQGELPPQEDP
SWQKDPDYQPPAKKTKKTKKS
23.
REST DNA
SEQ ID NO: 23
GGCATCCACAGCCATGAAGGAAGTGACCTAAGTGACAACATGTCA
GAGGGTAGTGATGATTCTGGATTGCATGGGGCTCGGCCAGTTCCA
CAAGAATCTAGCAGAAAAAATGCAAAGGAAGCCTTGGCAGTCAAA
GCGGCTAAGGGAGATTTTGTTTGTATCTTCTGTGATCGTTCTTTC
AGAAAGGGAAAAGATTACAGCAAACACCTCAATCGCCATTTGGTT
AATGTGTACTATCTTGAA
24.
REST peptide
SEQ ID NO: 24
GIHSHEGSDLSDNMSEGSDDSGLHGARPVPQESSRKNAKEALAVK
AAKGDFVCIFCDRSFRKGKDYSKHLNRHLVNVYYLE
25.
MGA DNA
SEQ ID NO: 25
CAGCCGTCCTGTACTCACATCTCTGCAGATGAAAAAGCAGCTGAA
AGGAGTCGAAAGGCTCCACCAATTCCTCTAAAACTGAAGCCTGAT
TACTGGAGTGACAAACTACAGAAAGAAGCAGAAGCGTTTGCTTAT
TATCGCCGGACACACACTGCCAATGAGCGGCGGCGGCGTGGTGAA
ATGAGGGATCTCTTTGAGAAATTAAAGATCACATTGGGATTACTT
CATTCTTCCAAGGTTTCCAAAAGTCTCATTCTTACTCGAGCCTTC
AGTGAAATTCAGGGACTAACAGATCAGGCAGACAAATTGATAGGA
CAGAAAAATCTCCTGACTCGAAAACGGAATATTCTGATACGGAAA
GTA
26.
MGA peptide
SEQ ID NO: 26
QPSCTHISADEKAAERSRKAPPIPLKLKPDYWSDKLQKEAEAFAY
YRRTHTANERRRRGEMRDLFEKLKITLGLLHSSKVSKSLILTRAF
SEIQGLTDQADKLIGQKNLLTRKRNILIRKV
27.
KLF10 DNA
SEQ ID NO: 27
ATGGCACCAGCGCCATCTACTGTACACTTCAAGTCACTCTCAGAT
ACTGCCAAACCTCACATTGCCGCACCTTTCAAAGAGGAAGAAAAG
AGCCCAGTATCTGCCCCCAAACTCCCCAAAGCTCAGGCAACAAGT
GTGATTCGTCATACAGCTGATGCCCAGCTATGTAACCACCAGACC
TGCCCAATGAAAGCAGCCAGCATCCTCAACTATCAGAACAATTCT
TTTA
28.
KLF10 peptide
SEQ ID NO: 28
MAPAPSTVHFKSLSDTAKPHIAAPFKEEEKSPVSAPKLPKAQATS
VIRHTADAQLCNHQTCPMKAASILNYQNNSFRRRTHLNVEAARKNI
GAAGAAGAACCCACCTAAATGTTGAGGCTGCAAGAAAGAACATA
29.
IRF2BP1 DNA
SEQ ID NO: 29
GCGTCTGTGCAGGCGTCCCGCCGCCAGTGGTGCTACCTGTGCGAC
CTGCCCAAGATGCCGTGGGCCATGGTGTGGGACTTCAGCGAGGCC
GTGTGTCGCGGCTGCGTGAACTTCGAGGGCGCGGACCGCATCGAA
CTGCTCATCGATGCCGCCCGCCAGCTCAAGCGCAGCCACGTGCTC
CCCGAGGGCCGCTCGCCCGGGCCCCCGGCCCTTAAGCACCCGGCC
ACCAAGGACCTGGCG
30.
IRF2BP1 peptide
SEQ ID NO: 30
ASVQASRRQWCYLCDLPKMPWAMVWDFSEAVCRGCVNFEGADRIE
LLIDAARQLKRSHVLPEGRSPGPPALKHPATKDLA
31.
MAX DNA
SEQ ID NO: 31
AGCGATAACGATGACATCGAGGTGGAGAGCGACGAAGAGCAACCG
AGGTTTCAATCTGCGGCTGACAAACGGGCTCATCATAATGCACTG
GAACGAAAACGTAGGGACCACATCAAAGACAGCTTTCACAGTTTG
CGGGACTCAGTCCCATCACTCCAAGGAGAGAAGGCATCCCGGGCC
CAAATCCTAGACAAAGCCACAGAATATATCCAGTATATGCGAAGG
AAAAACCACACACACCAGCAAGATATTGACGACCTCAAGCGGCAG
AATGCTCTTCTGGAGCAGCAAGTCCGTGCACTGGAGAAGGCGAGG
TCAAGTGCCCAACTGCAGACCAACTACCCCTCCTCAGACAACAGC
CTCTACACCAACGCCAAGGGCAGCACCATCTCTGCCTTCGATGGG
GGCTCGGACTCCAGCTCGGAGTCTGAGCCTGAAGAGCCCCAAAGC
AGGAAGAAGCTCCGGATGGAGGCCAGC
32.
MAX peptide
SEQ ID NO: 32
SDNDDIEVESDEEQPRFQSAADKRAHHNALERKRRDHIKDSFHSL
RDSVPSLQGEKASRAQILDKATEYIQYMRRKNHTHQQDIDDLKRQ
NALLEQQVRALEKARSSAQLQTNYPSSDNSLYTNAKGSTISAFDG
GSDSSSESEPEEPQSRKKLRMEAS
33.
IKZF5 DNA
SEQ ID NO: 33
AGCACTCCCAGCATAGGAAACAGCCAGCCAAGCACCCCAGCCCCA
GCCCTGCCGGTCCAGGACCCTCAGCTTCTGCACCACTGCCAGCAC
TGTGATATGTACTTTGCAGACAACATCCTTTACACTATTCATATG
GGATGTCATGGGTATGAAAATCCTTTTCAGTGTAATATATGTGGA
TGCAAATGTAAAAACAAGTATGATTTTGCCTGTCATTTTGCAAGA
GGGCAACATAACCAACAT
34.
IKZF5 peptide
SEQ ID NO: 34
STPSIGNSQPSTPAPALPVQDPQLLHHCQHCDMYFADNILYTIHM
GCHGYENPFQCNICGCKCKNKYDFACHFARGQHNQH
35.
RCOR1 DNA
SEQ ID NO: 35
CCCAATGGCAACAGCAGCAGCAACTCCTGGGAGGAAGGCAGCTCG
GGCTCGTCCAGCGACGAGGAGCACGGTGGCGGTGGCATGAGGGTC
GGACCCCAGTACCAGGCGGTGGTGCCCGACTTCGACCCCGCCAAA
CTGGCAAGACGCAGTCAAGAACGGGACAATCTTGGCATGTTGGTC
TGGTCACCCAATCAAAATCTGTCAGAAGCAAAGTTGGATGAATAC
ATTGCCATTGCCAAAGAAAAGCATGGGTACAACATGGAACAGGCT
CTTGGGATGCTCTTCTGGCATAAACATAATATCGAAAAGTCATTG
GCTGATTTGCCCAACTTTACCCCTTTCCCAGATGAGTGGACTGTG
GAAGATAAAGTCTTATTTGAGCAAGCCTTTAGTTTTCATGGGAAA
ACTTTTCATAGAATCCAACAAATGCTTCCAGATAAATCTATAGCA
AGTCTGGTGAAATTTTACTATTCTTGGAAGAAGACGAGGACTAAA
ACTAGTGTGATGGATCGCCATGCCCGGAAACAAAAACGGGAGCGG
GAGGAGAGCGAGGATGAACTGGAAGAGGCAAATGGAAACAATCCC
ATTGACATTGAGGTTGATCAAAACAAGGAAAGCAAAAAGGAGGTT
CCCCCTACTGAGACAGTTCCTCAGGTCAAAAAAGAAAAACATAGC
ACACAAGCTAAAAATAGAGCAAAAAGGAAACCTCCAAAAGGAATG
TTTCTTTCTCAAGAAGATGTGGAGGCTGTTTCTGCCAATGCCACT
GCTGCTACCACGGTGCTGAGACAACTAGACATGGAATTGGTTTCA
GTCAAACGACAGATCCAGAATATTAAACAGACAAACAGTGCTCTC
AAAGAAAAACTTGATGGTGGAATAGAACCATATCGACTTCCAGAG
GTCATTCAGAAATGTAAT
36.
RCOR1 peptide
SEQ ID NO: 36
PNGNSSSNSWEEGSSGSSSDEEHGGGGMRVGPQYQAVVPDFDPAK
LARRSQERDNLGMLVWSPNQNLSEAKLDEYIAIAKEKHGYNMEQA
LGMLFWHKHNIEKSLADLPNFTPFPDEWTVEDKVLFEQAFSFHGK
TFHRIQQMLPDKSIASLVKFYYSWKKTRTKTSVMDRHARKQKRER
EESEDELEEANGNNPIDIEVDQNKESKKEVPPTETVPQVKKEKHS
TQAKNRAKRKPPKGMFLSQEDVEAVSANATAATTVLRQLDMELVS
VKRQIQNIKQTNSALKEKLDGGIEPYRLPEVIQKCN
37.
ZIM3(KRAB)-MeCP2 DNA
SEQ ID NO: 37
ATGAACAATTCCCAGGGAAGAGTGACCTTCGAGGATGTCACTGTG
AACTTCACCCAGGGGGAGTGGCAGCGGCTGAATCCCG
AACAGAGAAACTTGTACAG
GGATGTGATGCTGGAGAATTACAGCAACCTTGTCTCTGTGGGACA
AGGGGAAACCACCAAACCCGATGTGATCTTGAGGTTGGAACAAGG
AAAGGAGCCGTGGTTGGAGGAAGAGGAAGTGCTGGGAAGTGGCCG
TGCAGAAAAAAATGGGGACATTGGAGGGCAGATTTGGAAGCCAAA
GGATGTGAAAGAGAGTCTCGGCTCTGGCAGCGCTTCTGCTGGAGA
AGCCTCAGTGCAGGTGAAAAGGGTGCTGGAAAAATCCCCCGGCAA
ACTCCTCGTGAAGATGCCCTTCCAGGCTTCCCCTGGCGGAAAAGG
TGAAGGGGGTGGCGCAACCACATCTGCCCAGGTCATGGTCATCAA
GCGACCTGGAAGGAAAAGAAAGGCCGAGGCTGACCCTCAGGCCAT
TCCAAAGAAACGGGGACGCAAGCCAGGGTCCGTGGTCGCAGCTGC
AGCAGCTGAGGCTAAGAAAAAGGCAGTGAAGGAAAGCTCCATCCG
CAGTGTGCAGGAGACTGTCCTGCCCATCAAGAAGAGGAAGACTAG
GGAGACCGTGTCCATCGAGGTCAAAGAAGTGGTCAAGCCCCTGCT
CGTGTCCACCCTGGGCGAAAAATCTGGAAAGGGGCTCAAAACATG
CAAGTCACCTGGACGGAAAAGCAAGGAGTCTAGTCCAAAGGGGCG
CTCAAGCTCCGCTTCTAGTCCCCCTAAAAAGGAACACCATCACCA
TCACCATCACGCCGAGTCTCCTAAGGCTCCTATGCCACTGCTCCC
ACCACCTCCACCACCTGAGCCACAGTCAAGCGAAGACCCCATCAG
CCCACCCGAGCCTCAGGATCTGTCCTCTAGTATTTGCAAAGAGGA
AAAGATGCCCAGAGCAGGCAGCCTGGAGAGTGATGGCTGTCCAAA
AGAACCCGCCAAGACCCAGCCTATGGTGGCAGCCGCTGCAACTAC
CACCACAACCACAACTACCACAGTGGCCGAAAAATACAAGCATCG
CGGCGAGGGCGAACGAAAGGACATTGTGTCAAGCTCCATGCCCAG
ACCTAACCGGGAGGAACCAGTCGATAGTAGGACACCCGTGACTGA
GAGAGTCTCA
38.
ZIM3(KRAB)-MeCP2 peptide
SEQ ID NO: 38
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVS
39.
KOX1(KRAB)-MeCP2(t) DNA
SEQ ID NO: 39
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTG
40.
KOX1(KRAB)-MeCP2(t) peptide
SEQ ID NO: 40
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEV
41.
ZIM3(KRAB)-MAX DNA
SEQ ID NO: 41
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGC
42.
ZIM3(KRAB)-MAX peptide
SEQ ID NO: 42
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEAS
43.
KRBOX1(KRAB)-MAX DNA
SEQ ID NO: 43
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGC
44.
KRBOX1(KRAB)-MAX peptide
SEQ ID NO: 44
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEAS
45.
KOX1(KRAB)-MAX DNA
SEQ ID NO: 45
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAAGCGAT
AACGATGACATCGAGGTGGAGAGCGACGAAGAGCAACCGAGGTTT
CAATCTGCGGCTGACAAACGGGCTCATCATAATGCACTGGAACGA
AAACGTAGGGACCACATCAAAGACAGCTTTCACAGTTTGCGGGAC
TCAGTCCCATCACTCCAAGGAGAGAAGGCATCCCGGGCCCAAATC
CTAGACAAAGCCACAGAATATATCCAGTATATGCGAAGGAAAAAC
CACACACACCAGCAAGATATTGACGACCTCAAGCGGCAGAATGCT
CTTCTGGAGCAGCAAGTCCGTGCACTGGAGAAGGCGAGGTCAAGT
GCCCAACTGCAGACCAACTACCCCTCCTCAGACAACAGCCTCTAC
ACCAACGCCAAGGGCAGCACCATCTCTGCCTTCGATGGGGGCTCG
GACTCCAGCTCGGAGTCTGAGCCTGAAGAGCCCCAAAGCAGGAAG
AAGCTCCGGATGGAGGCCAGC
46.
KOX(KRAB)-MAX peptide
SEQ ID NO: 46
RTLVTFKDVFVDFTREEWKLLDTAQQILYRNVMLENYKNLVSLGY
QLTKPDVILRLEKGEEPWLVGSGSASAGSDNDDIEVESDEEQPRF
QSAADKRAHHNALERKRRDHIKDSFHSLRDSVPSLQGEKASRAQI
LDKATEYIQYMRRKNHTHQQDIDDLKRQNALLEQQVRALEKARSS
AQLQTNYPSSDNSLYTNAKGSTISAFDGGSDSSSESEPEEPQSRK
KLRMEAS
47.
ZIM3(KRAB)-IRF2BP1 DNA
SEQ ID NO: 47
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGCGTCTGTGCAGGCGTCCCGCCGCCAGTGGTGCTAC
CTGTGCGACCTGCCCAAGATGCCGTGGGCCATGGTGTGGGACTTC
AGCGAGGCCGTGTGTCGCGGCTGCGTGAACTTCGAGGGCGCGGAC
CGCATCGAACTGCTCATCGATGCCGCCCGCCAGCTCAAGCGCAGC
CACGTGCTCCCCGAGGGCCGCTCGCCCGGGCCCCCGGCCCTTAAG
CACCCGGCCACCAAGGACCTGGCG
48.
ZIM3(KRAB)-IRF2BP1 peptide
SEQ ID NO: 48
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGASVQASRRQWCYLCDLPKMPWAMVWDF
SEAVCRGCVNFEGADRIELLIDAARQLKRSHVLPEGRSPGPPALK
HPATKDLA
49.
ZIM3(KRAB)-ZIM3(KRAB) DNA
SEQ ID NO: 49
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAATGAACAATTCCCAGGGAAGAGTGACCTTCGAGGAT
GTCACTGTGAACTTCACCCAGGGGGAGTGGCAGCGGCTGAATCCC
GAACAGAGAAACTTGTACAGGGATGTGATGCTGGAGAATTACAGC
AACCTTGTCTCTGTGGGACAAGGGGAAACCACCAAACCCGATGTG
ATCTTGAGGTTGGAACAAGGAAAGGAGCCGTGGTTGGAGGAAGAG
GAAGTGCTGGGAAGTGGCCGTGCAGAAAAAAATGGGGACATTGGA
GGGCAGATTTGGAAGCCAAAGGATGTGAAAGAGAGTCTC
50.
ZIM3(KRAB)-ZIM3(KRAB) peptide
SEQ ID NO: 50
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGMNNSQGRVTFEDVTVNFTQGEWQRLNP
EQRNLYRDVMLENYSNLVSVGQGETTKPDVILRLEQGKEPWLEEE
EVLGSGRAEKNGDIGGQIWKPKDVKESL
51.
KRBOX1(KRAB)-CTCF DNA
SEQ ID NO: 51
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAGTTGTAAATATGGAGGAACAG
CCCATAAACATAGGAGAACTTCAGCTTGTTCAAGTACCTGTTCCT
GTGACTGTACCTGTTGCTACCACTTCAGTAGAAGAACTTCAGGGG
GCTTATGAAAATGAAGTGTCTAAAGAGGGCCTTGCGGAAAGTGAA
CCCATGATATGCCACACCCTACCTTTGCCTGAAGGGTTTCAGGTG
GTTAAAGTGGGGGCCAATGGAGAGGTGGAGACACTAGAACAAGGG
GAACTTCCACCCCAGGAAGATCCTAGTTGGCAAAAAGACCCAGAC
TATCAGCCACCAGCCAAAAAAACAAAGAAAACCAAAAAGAGC
52.
KRBOX1(KRAB)-CTCF peptide
SEQ ID NO: 52
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGVVNMEEQPINIGELQLVQVPVPVTVPVATTSVEELQG
AYENEVSKEGLAESEPMICHTLPLPEGFQVVKVGANGEVETLEQG
ELPPQEDPSWQKDPDYQPPAKKTKKTKKS
53.
ZIM3(KRAB)-ZNF554(KRAB) DNA
SEQ ID NO: 53
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGATTTTCCCAAGAGGAGAGAATGGCTGCTGGGTACCTG
CCCCGCTGGTCCCAGGAATTAGTAACCTTTGAGGACGTGTCCATG
GACTTCTCCCAGGAGGAGTGGGAGTTGCTGGAGCCTGCTCAGAAG
AACCTGTACAGAGAGGTGATGCTGGAGAACTACAGGAACGTGGTC
TCCCTGGAAGCCTTGAAGAACCAATGTACTGATGTGGGGATTAAA
GAGGGTCCACTTTCCCCAGCACAAACCTCACAAGTCACTAGTCTT
TCCTCATGGACGGGGTATTTACTTTTTCAACCAGTGGCTTCTTCC
CACTTGGAGCAAAGAGAAGCCCTGTGGATAGAGGAAAAAGGAACT
CCTCAAGCCTCCTGTTCAGATTGGATGACTGTACTAAGAAACCAA
GACTCAACTTACAAGAAGGTGGCTTTGCAGGAG
54.
ZIM3(KRAB)-ZNF554(KRAB) peptide
SEQ ID NO: 54
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGFSQEERMAAGYLPRWSQELVTFEDVSM
DFSQEEWELLEPAQKNLYREVMLENYRNVVSLEALKNQCTDVGIK
EGPLSPAQTSQVTSLSSWTGYLLFQPVASSHLEQREALWIEEKGT
PQASCSDWMTVLRNQDSTYKKVALQE
55.
KRBOX1(KRAB)-MeCP2 DNA
SEQ ID NO: 55
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAGAAGCCTCAGTGCAGGTGAAA
AGGGTGCTGGAAAAATCCCCCGGCAAACTCCTCGTGAAGATGCCC
TTCCAGGCTTCCCCTGGCGGAAAAGGTGAAGGGGGTGGCGCAACC
ACATCTGCCCAGGTCATGGTCATCAAGCGACCTGGAAGGAAAAGA
AAGGCCGAGGCTGACCCTCAGGCCATTCCAAAGAAACGGGGACGC
AAGCCAGGGTCCGTGGTCGCAGCTGCAGCAGCTGAGGCTAAGAAA
AAGGCAGTGAAGGAAAGCTCCATCCGCAGTGTGCAGGAGACTGTC
CTGCCCATCAAGAAGAGGAAGACTAGGGAGACCGTGTCCATCGAG
GTCAAAGAAGTGGTCAAGCCCCTGCTCGTGTCCACCCTGGGCGAA
AAATCTGGAAAGGGGCTCAAAACATGCAAGTCACCTGGACGGAAA
AGCAAGGAGTCTAGTCCAAAGGGGCGCTCAAGCTCCGCTTCTAGT
CCCCCTAAAAAGGAACACCATCACCATCACCATCACGCCGAGTCT
CCTAAGGCTCCTATGCCACTGCTCCCACCACCTCCACCACCTGAG
CCACAGTCAAGCGAAGACCCCATCAGCCCACCCGAGCCTCAGGAT
CTGTCCTCTAGTATTTGCAAAGAGGAAAAGATGCCCAGAGCAGGC
AGCCTGGAGAGTGATGGCTGTCCAAAAGAACCCGCCAAGACCCAG
CCTATGGTGGCAGCCGCTGCAACTACCACCACAACCACAACTACC
ACAGTGGCCGAAAAATACAAGCATCGCGGCGAGGGCGAACGAAAG
GACATTGTGTCAAGCTCCATGCCCAGACCTAACCGGGAGGAACCA
GTCGATAGTAGGACACCCGTGACTGAGAGAGTCTCA
56.
KRBOX1(KRAB)-MeCP2 peptide
SEQ ID NO: 56
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGEASVQVKRVLEKSPGKLLVKMPFQASPGGKGEGGGAT
TSAQVMVIKRPGRKRKAEADPQAIPKKRGRKPGSVVAAAAAEAKK
KAVKESSIRSVQETVLPIKKRKTRETVSIEVKEVVKPLLVSTLGE
KSGKGLKTCKSPGRKSKESSPKGRSSSASSPPKKEHHHHHHHAES
PKAPMPLLPPPPPPEPQSSEDPISPPEPQDLSSSICKEEKMPRAG
SLESDGCPKEPAKTQPMVAAAATTTTTTTTTVAEKYKHRGEGERK
DIVSSSMPRPNREEPVDSRTPVTERVS
57.
ZIM3(KRAB)-RYBP DNA
SEQ ID NO: 57
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGATCCTCCTAGTGAAGCAAACAGCATACAGTCTGCA
AATGCTACAACAAAGACCAGCGAAACAAATCACACCTCAAGGCCC
CGGCTGAAAAACGTGGACAGGAGCACTGCACAGCAGTTGGCAGTA
ACTGTGGGCAACGTCACCGTCATTATCACAGACTTTAAGGAAAAG
ACTCGCTCCTCATCGACATCCTCATCCACAGTGACCTCCAGTGCA
GGGTCAGAACAGCAGAACCAGAGCAGCTCGGGGTCAGAGAGCACA
GACAAGGGCTCCTCCCGTTCCTCCACGCCAAAGGGCGACATG
58.
ZIM3(KRAB)-RYBP peptide
SEQ ID NO: 58
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGDPPSEANSIQSANATTKTSETNHTSRP
RLKNVDRSTAQQLAVTVGNVTVIITDFKEKTRSSSTSSSTVTSSA
GSEQQNQSSSGSESTDKGSSRSSTPKGDM
59.
ZIM3(KRAB)-KLF10 DNA
SEQ ID NO: 59
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAATGGCACCAGCGCCATCTACTGTACACTTCAAGTCA
CTCTCAGATACTGCCAAACCTCACATTGCCGCACCTTTCAAAGAG
GAAGAAAAGAGCCCAGTATCTGCCCCCAAACTCCCCAAAGCTCAG
GCAACAAGTGTGATTCGTCATACAGCTGATGCCCAGCTATGTAAC
CACCAGACCTGCCCAATGAAAGCAGCCAGCATCCTCAACTATCAG
AACAATTCTTTTAGAAGAAGAACCCACCTAAATGTTGAGGCTGCA
AGAAAGAACATA
60.
ZIM3(KRAB)-KLF10 peptide
SEQ ID NO: 60
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGMAPAPSTVHFKSLSDTAKPHIAAPFKE
EEKSPVSAPKLPKAQATSVIRHTADAQLCNHQTCPMKAASILNYQ
NNSFRRRTHLNVEAARKNI
61.
KRBOX1(KRAB)-ZIM3(KRAB) DNA
SEQ ID NO: 61
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAATGAACAATTCCCAGGGAAGA
GTGACCTTCGAGGATGTCACTGTGAACTTCACCCAGGGGGAGTGG
CAGCGGCTGAATCCCGAACAGAGAAACTTGTACAGGGATGTGATG
CTGGAGAATTACAGCAACCTTGTCTCTGTGGGACAAGGGGAAACC
ACCAAACCCGATGTGATCTTGAGGTTGGAACAAGGAAAGGAGCCG
TGGTTGGAGGAAGAGGAAGTGCTGGGAAGTGGCCGTGCAGAAAAA
AATGGGGACATTGGAGGGCAGATTTGGAAGCCAAAGGATGTGAAA
GAGAGTCTC
62.
KRBOX1(KRAB)-ZIM3(KRAB) peptide
SEQ ID NO: 62
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGMNNSQGRVTFEDVTVNFTQGEWQRLNPEQRNLYRDVM
LENYSNLVSVGQGETTKPDVILRLEQGKEPWLEEEEVLGSGRAEK
NGDIGGQIWKPKDVKESL
63.
ZIM3(KRAB)-MAX-MeCP2(t) DNA
SEQ ID NO: 63
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGAACCACATCCACCCAGGTCATGGTGATCAAACGCCCC
GGCAGGAAGCGAAAAGCTGAGGCCGACCCTCAGGCCATTCCCAAG
AAACGGGGCCGAAAGCCGGGGAGTGTGGTGGCAGCCGCTGCCGCC
GAGGCCAAAAAGAAAGCCGTGAAGGAGTCTTCTATCCGATCTGTG
CAGGAGACCGTACTCCCCATCAAGAAGCGCAAGACCCGGGAGACC
GTCAGCATCGAGGTCAAGGAAGTG
64.
ZIM3(KRAB)-MAX-MeCP2(t) peptide
SEQ ID NO: 64
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGTTSTQVMVIKRPGRKRKAEADPQAIPKKRGRKPGSVVAAAAA
EAKKKAVKESSIRSVQETVLPIKKRKTRETVSIEVKEV
65.
KOX(KRAB)-MeCP2(t)-MeCP2(t) DNA
SEQ ID NO: 65
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTGGGCAGCGCTTCTGCTGGAACCACATCCACCCAGGTC
ATGGTGATCAAACGCCCCGGCAGGAAGCGAAAAGCTGAGGCCGAC
CCTCAGGCCATTCCCAAGAAACGGGGCCGAAAGCCGGGGAGTGTG
GTGGCAGCCGCTGCCGCCGAGGCCAAAAAGAAAGCCGTGAAGGAG
TCTTCTATCCGATCTGTGCAGGAGACCGTACTCCCCATCAAGAAG
CGCAAGACCCGGGAGACCGTCAGCATCGAGGTCAAGGAAGTG
66.
KOX(KRAB)-MeCP2(t)-MeCP2(t) peptide
SEQ ID NO: 66
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEVGSASAGTTSTQVMVIKRPGRKRKAEAD
PQAIPKKRGRKPGSVVAAAAAEAKKKAVKESSIRSVQETVLPIKK
RKTRETVSIEVKEV
67.
KOX1(KRAB)-MeCP2(t)-KOX1(KRAB) DNA
SEQ ID NO: 67
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTGGGCAGCGCTTCTGCTGGACGGACACTGGTGACCTTC
AAGGATGTGTTTGTGGACTTCACCAGGGAGGAGTGGAAGCTGCTG
GACACTGCTCAGCAGATCCTGTACAGAAATGTGATGCTGGAGAAC
TATAAGAACCTGGTTTCCTTGGGTTATCAGCTTACTAAGCCAGAT
GTGATCCTCCGGTTGGAGAAGGGAGAAGAGCCCTGGCTGGTG
68.
KOX1(KRAB)-MeCP2(t)-KOX1(KRAB) peptide
SEQ ID NO: 68
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEVGSASAGRTLVTFKDVFVDFTREEWKLL
DTAQQILYRNVMLENYKNLVSLGYQLTKPDVILRLEKGEEPWLV
69.
ZIM3(KRAB)-MAX-IRF2BP1 DNA
SEQ ID NO: 69
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGAGCGTCTGTGCAGGCGTCCCGCCGCCAGTGGTGCTAC
CTGTGCGACCTGCCCAAGATGCCGTGGGCCATGGTGTGGGACTTC
AGCGAGGCCGTGTGTCGCGGCTGCGTGAACTTCGAGGGCGCGGAC
CGCATCGAACTGCTCATCGATGCCGCCCGCCAGCTCAAGCGCAGC
CACGTGCTCCCCGAGGGCCGCTCGCCCGGGCCCCCGGCCCTTAAG
CACCCGGCCACCAAGGACCTGGCG
70.
ZIM3(KRAB)-MAX-IRF2BP1 peptide
SEQ ID NO: 70
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGASVQASRRQWCYLCDLPKMPWAMVWDFSEAVCRGCVNFEGAD
RIELLIDAARQLKRSHVLPEGRSPGPPALKHPATKDLA
71.
KOX1(KRAB)-MeCP2(t)-ZNF264(KRAB) DNA
SEQ ID NO: 71
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTGGGCAGCGCTTCTGCTGGAGCGGCAGCGGTGCTGACG
GACCGGGCCCAGGTGTCTGTGACCTTTGATGATGTGGCTGTGACT
TTCACCAAGGAGGAGTGGGGGCAGCTGGACCTAGCTCAGCGGACC
CTGTACCAGGAGGTGATGCTGGAAAACTGTGGGCTCCTGGTGTCT
CTGGGGTGTCCTGTTCCCAAAGCTGAGCTGATCTGCCACCTAGAG
CATGGGCAGGAGCCATGGACCAGGAAGGAAGACCTCTCCCAAGAC
ACCTGTCCAGGCGACAAAGGAAAACCTAAGACCACAGAACCTACC
ACTTGTGAGCCAGCCTTGTCAGAG
72.
KOX1(KRAB)-MeCP2(t)-ZNF264(KRAB) peptide
SEQ ID NO: 72
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEVGSASAGAAAVLTDRAQVSVTFDDVAVT
FTKEEWGQLDLAQRTLYQEVMLENCGLLVSLGCPVPKAELICHLE
HGQEPWTRKEDLSQDTCPGDKGKPKTTEPTTCEPALSE
73.
KRBOX1(KRAB)-MAX-MeCP2(t) DNA
SEQ ID NO: 73
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGAACCACATCCACCCAGGTCATG
GTGATCAAACGCCCCGGCAGGAAGCGAAAAGCTGAGGCCGACCCT
CAGGCCATTCCCAAGAAACGGGGCCGAAAGCCGGGGAGTGTGGTG
GCAGCCGCTGCCGCCGAGGCCAAAAAGAAAGCCGTGAAGGAGTCT
TCTATCCGATCTGTGCAGGAGACCGTACTCCCCATCAAGAAGCGC
AAGACCCGGGAGACCGTCAGCATCGAGGTCAAGGAAGTG
74.
KRBOX1(KRAB)-MAX-MeCP2(t) peptide
SEQ ID NO: 74
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGTTSTQVM
VIKRPGRKRKAEADPQAIPKKRGRKPGSVVAAAAAEAKKKAVKES
SIRSVQETVLPIKKRKTRETVSIEVKEV
75.
ZIM3(KRAB)-MeCP2-RYBP DNA
SEQ ID NO: 75
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGAGATCCT
CCTAGTGAAGCAAACAGCATACAGTCTGCAAATGCTACAACAAAG
ACCAGCGAAACAAATCACACCTCAAGGCCCCGGCTGAAAAACGTG
GACAGGAGCACTGCACAGCAGTTGGCAGTAACTGTGGGCAACGTC
ACCGTCATTATCACAGACTTTAAGGAAAAGACTCGCTCCTCATCG
ACATCCTCATCCACAGTGACCTCCAGTGCAGGGTCAGAACAGCAG
AACCAGAGCAGCTCGGGGTCAGAGAGCACAGACAAGGGCTCCTCC
CGTTCCTCCACGCCAAAGGGCGACATG
76.
ZIM3(KRAB)-MeCP2-RYBP peptide
SEQ ID NO: 76
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGDP
PSEANSIQSANATTKTSETNHTSRPRLKNVDRSTAQQLAVTVGNV
TVIITDFKEKTRSSSTSSSTVTSSAGSEQQNQSSSGSESTDKGSS
RSSTPKGDM
77.
ZIM3(KRAB)-MAX-ZNF554(KRAB) DNA
SEQ ID NO: 77
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGATTTTCCCAAGAGGAGAGAATGGCTGCTGGGTACCTG
CCCCGCTGGTCCCAGGAATTAGTAACCTTTGAGGACGTGTCCATG
GACTTCTCCCAGGAGGAGTGGGAGTTGCTGGAGCCTGCTCAGAAG
AACCTGTACAGAGAGGTGATGCTGGAGAACTACAGGAACGTGGTC
TCCCTGGAAGCCTTGAAGAACCAATGTACTGATGTGGGGATTAAA
GAGGGTCCACTTTCCCCAGCACAAACCTCACAAGTCACTAGTCTT
TCCTCATGGACGGGGTATTTACTTTTTCAACCAGTGGCTTCTTCC
CACTTGGAGCAAAGAGAAGCCCTGTGGATAGAGGAAAAAGGAACT
CCTCAAGCCTCCTGTTCAGATTGGATGACTGTACTAAGAAACCAA
GACTCAACTTACAAGAAGGTGGCTTTGCAGGAG
78.
ZIM3(KRAB)-MAX-ZNF554(KRAB) peptide
SEQ ID NO: 78
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGFSQEERMAAGYLPRWSQELVTFEDVSMDFSQEEWELLEPAQK
NLYREVMLENYRNVVSLEALKNQCTDVGIKEGPLSPAQTSQVTSL
SSWTGYLLFQPVASSHLEQREALWIEEKGTPQASCSDWMTVLRNQ
DSTYKKVALQE
79.
ZIM3(KRAB)-MAX-KOX1(KRAB) DNA
SEQ ID NO: 79
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGACGGACACTGGTGACCTTCAAGGATGTGTTTGTGGAC
TTCACCAGGGAGGAGTGGAAGCTGCTGGACACTGCTCAGCAGATC
CTGTACAGAAATGTGATGCTGGAGAACTATAAGAACCTGGTTTCC
TTGGGTTATCAGCTTACTAAGCCAGATGTGATCCTCCGGTTGGAG
AAGGGAGAAGAGCCCTGGCTGGTG
80.
ZIM3(KRAB)-MAX-KOX1(KRAB) peptide
SEQ ID NO: 80
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGRTLVTFKDVFVDFTREEWKLLDTAQQILYRNVMLENYKNLVS
LGYQLTKPDVILRLEKGEEPWLV
81.
ZIM3(KRAB)-MeCP2-KRBOX1(KRAB) DNA
SEQ ID NO: 81
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGAATGACA
GCTGTGTCCTTAACAACCAGGCCCCAGGAATCAGTGGCTTTTGAG
GACGTGGCTGTGTACTTCACTACGAAGGAATGGGCCATCATGGTG
CCTGCCGAGAGGGCCTTGTACAGGGATGTGATGCTGGAGAACTAT
GAGGCTGTGGCCTTTGTAGTGCCACCCACTTCCAAACCAGCTTTG
GTCTCTCATCTGGAGCAAGGGAAAGAGTCCTGTTTCACCCAGCCA
CAGGGAGTCCTAAGCAGGAATGACTGGAGAGCAGGCTGG
82.
ZIM3(KRAB)-MeCP2-KRBOX1(KRAB) peptide
SEQ ID NO: 82
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGMT
AVSLTTRPQESVAFEDVAVYFTTKEWAIMVPAERALYRDVMLENY
EAVAFVVPPTSKPALVSHLEQGKESCFTQPQGVLSRNDWRAGW
83.
KRBOX1(KRAB)-MAX-ZIM3(KRAB) DNA
SEQ ID NO: 83
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGAATGAACAATTCCCAGGGAAGA
GTGACCTTCGAGGATGTCACTGTGAACTTCACCCAGGGGGAGTGG
CAGCGGCTGAATCCCGAACAGAGAAACTTGTACAGGGATGTGATG
CTGGAGAATTACAGCAACCTTGTCTCTGTGGGACAAGGGGAAACC
ACCAAACCCGATGTGATCTTGAGGTTGGAACAAGGAAAGGAGCCG
TGGTTGGAGGAAGAGGAAGTGCTGGGAAGTGGCCGTGCAGAAAAA
AATGGGGACATTGGAGGGCAGATTTGGAAGCCAAAGGATGTGAAA
GAGAGTCTC
84.
KRBOX1(KRAB)-MAX-ZIM3(KRAB) peptide
SEQ ID NO: 84
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGMNNSQGR
VTFEDVTVNFTQGEWQRLNPEQRNLYRDVMLENYSNLVSVGQGET
TKPDVILRLEQGKEPWLEEEEVLGSGRAEKNGDIGGQIWKPKDVK
ESL
85.
ZIM3(KRAB)-MeCP2-MeCP2(t) DNA
SEQ ID NO: 85
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTG
86.
ZIM3(KRAB)-MeCP2-MeCP2(t) peptide
SEQ ID NO: 86
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGTT
STQVMVIKRPGRKRKAEADPQAIPKKRGRKPGSVVAAAAAEAKKK
AVKESSIRSVQETVLPIKKRKTRETVSIEVKEV
87.
ZIM3(KRAB)-MAX-ZNF264(KRAB) DNA
SEQ ID NO: 87
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGAGCGGCAGCGGTGCTGACGGACCGGGCCCAGGTGTCT
GTGACCTTTGATGATGTGGCTGTGACTTTCACCAAGGAGGAGTGG
GGGCAGCTGGACCTAGCTCAGCGGACCCTGTACCAGGAGGTGATG
CTGGAAAACTGTGGGCTCCTGGTGTCTCTGGGGTGTCCTGTTCCC
AAAGCTGAGCTGATCTGCCACCTAGAGCATGGGCAGGAGCCATGG
ACCAGGAAGGAAGACCTCTCCCAAGACACCTGTCCAGGCGACAAA
GGAAAACCTAAGACCACAGAACCTACCACTTGTGAGCCAGCCTTG
TCAGAG
88.
ZIM3(KRAB)-MAX-ZNF264(KRAB) peptide
SEQ ID NO: 88
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGAAAVLTDRAQVSVTFDDVAVTFTKEEWGQLDLAQRTLYQEVM
LENCGLLVSLGCPVPKAELICHLEHGQEPWTRKEDLSQDTCPGDK
GKPKTTEPTTCEPALSE
89.
ZIM3(KRAB)-MeCP2-ZIM3(KRAB) DNA
SEQ ID NO: 89
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGAATGAAC
AATTCCCAGGGAAGAGTGACCTTCGAGGATGTCACTGTGAACTTC
ACCCAGGGGGAGTGGCAGCGGCTGAATCCCGAACAGAGAAACTTG
TACAGGGATGTGATGCTGGAGAATTACAGCAACCTTGTCTCTGTG
GGACAAGGGGAAACCACCAAACCCGATGTGATCTTGAGGTTGGAA
CAAGGAAAGGAGCCGTGGTTGGAGGAAGAGGAAGTGCTGGGAAGT
GGCCGTGCAGAAAAAAATGGGGACATTGGAGGGCAGATTTGGAAG
CCAAAGGATGTGAAAGAGAGTCTC
90.
ZIM3(KRAB)-MeCP2-ZIM3(KRAB) peptide
SEQ ID NO: 90
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGMN
NSQGRVTFEDVTVNFTQGEWQRLNPEQRNLYRDVMLENYSNLVSV
GQGETTKPDVILRLEQGKEPWLEEEEVLGSGRAEKNGDIGGQIWK
PKDVKESL
91.
KRBOX1(KRAB)-MAX-MeCP2 DNA
SEQ ID NO: 91
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGAGAAGCCTCAGTGCAGGTGAAA
AGGGTGCTGGAAAAATCCCCCGGCAAACTCCTCGTGAAGATGCCC
TTCCAGGCTTCCCCTGGCGGAAAAGGTGAAGGGGGTGGCGCAACC
ACATCTGCCCAGGTCATGGTCATCAAGCGACCTGGAAGGAAAAGA
AAGGCCGAGGCTGACCCTCAGGCCATTCCAAAGAAACGGGGACGC
AAGCCAGGGTCCGTGGTCGCAGCTGCAGCAGCTGAGGCTAAGAAA
AAGGCAGTGAAGGAAAGCTCCATCCGCAGTGTGCAGGAGACTGTC
CTGCCCATCAAGAAGAGGAAGACTAGGGAGACCGTGTCCATCGAG
GTCAAAGAAGTGGTCAAGCCCCTGCTCGTGTCCACCCTGGGCGAA
AAATCTGGAAAGGGGCTCAAAACATGCAAGTCACCTGGACGGAAA
AGCAAGGAGTCTAGTCCAAAGGGGCGCTCAAGCTCCGCTTCTAGT
CCCCCTAAAAAGGAACACCATCACCATCACCATCACGCCGAGTCT
CCTAAGGCTCCTATGCCACTGCTCCCACCACCTCCACCACCTGAG
CCACAGTCAAGCGAAGACCCCATCAGCCCACCCGAGCCTCAGGAT
CTGTCCTCTAGTATTTGCAAAGAGGAAAAGATGCCCAGAGCAGGC
AGCCTGGAGAGTGATGGCTGTCCAAAAGAACCCGCCAAGACCCAG
CCTATGGTGGCAGCCGCTGCAACTACCACCACAACCACAACTACC
ACAGTGGCCGAAAAATACAAGCATCGCGGCGAGGGCGAACGAAAG
GACATTGTGTCAAGCTCCATGCCCAGACCTAACCGGGAGGAACCA
GTCGATAGTAGGACACCCGTGACTGAGAGAGTCTCA
92.
KRBOX1(KRAB)-MAX-MeCP2 peptide
SEQ ID NO: 92
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGEASVQVK
RVLEKSPGKLLVKMPFQASPGGKGEGGGATTSAQVMVIKRPGRKR
KAEADPQAIPKKRGRKPGSVVAAAAAEAKKKAVKESSIRSVQETV
LPIKKRKTRETVSIEVKEVVKPLLVSTLGEKSGKGLKTCKSPGRK
SKESSPKGRSSSASSPPKKEHHHHHHHAESPKAPMPLLPPPPPPE
PQSSEDPISPPEPQDLSSSICKEEKMPRAGSLESDGCPKEPAKTQ
PMVAAAATTTTTTTTTVAEKYKHRGEGERKDIVSSSMPRPNREEP
VDSRTPVTERVS
93.
KRBOX1(KRAB)-MAX-ZNF554(KRAB) DNA
SEQ ID NO: 93
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGATTTTCCCAAGAGGAGAGAATG
GCTGCTGGGTACCTGCCCCGCTGGTCCCAGGAATTAGTAACCTTT
GAGGACGTGTCCATGGACTTCTCCCAGGAGGAGTGGGAGTTGCTG
GAGCCTGCTCAGAAGAACCTGTACAGAGAGGTGATGCTGGAGAAC
TACAGGAACGTGGTCTCCCTGGAAGCCTTGAAGAACCAATGTACT
GATGTGGGGATTAAAGAGGGTCCACTTTCCCCAGCACAAACCTCA
CAAGTCACTAGTCTTTCCTCATGGACGGGGTATTTACTTTTTCAA
CCAGTGGCTTCTTCCCACTTGGAGCAAAGAGAAGCCCTGTGGATA
GAGGAAAAAGGAACTCCTCAAGCCTCCTGTTCAGATTGGATGACT
GTACTAAGAAACCAAGACTCAACTTACAAGAAGGTGGCTTTGCAG
GAG
94.
KRBOX1(KRAB)-MAX-ZNF554(KRAB) peptide
SEQ ID NO: 94
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGFSQEERM
AAGYLPRWSQELVTFEDVSMDESQEEWELLEPAQKNLYREVMLEN
YRNVVSLEALKNQCTDVGIKEGPLSPAQTSQVTSLSSWTGYLLFQ
PVASSHLEQREALWIEEKGTPQASCSDWMTVLRNQDSTYKKVALQ
E
95.
ZIM(KRAB)-MeCP2-IRFBP1 DNA
SEQ ID NO: 95
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGAGCGTCT
GTGCAGGCGTCCCGCCGCCAGTGGTGCTACCTGTGCGACCTGCCC
AAGATGCCGTGGGCCATGGTGTGGGACTTCAGCGAGGCCGTGTGT
CGCGGCTGCGTGAACTTCGAGGGCGCGGACCGCATCGAACTGCTC
ATCGATGCCGCCCGCCAGCTCAAGCGCAGCCACGTGCTCCCCGAG
GGCCGCTCGCCCGGGCCCCCGGCCCTTAAGCACCCGGCCACCAAG
GACCTGGCG
96.
ZIM(KRAB)-MeCP2-IRFBP1 peptide
SEQ ID NO: 96
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGAS
VQASRRQWCYLCDLPKMPWAMVWDFSEAVCRGCVNFEGADRIELL
IDAARQLKRSHVLPEGRSPGPPALKHPATKDLA
97.
ZIM3(KRAB)-MAX-CTCF DNA
SEQ ID NO: 97
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGAGTTGTAAATATGGAGGAACAGCCCATAAACATAGGA
GAACTTCAGCTTGTTCAAGTACCTGTTCCTGTGACTGTACCTGTT
GCTACCACTTCAGTAGAAGAACTTCAGGGGGCTTATGAAAATGAA
GTGTCTAAAGAGGGCCTTGCGGAAAGTGAACCCATGATATGCCAC
ACCCTACCTTTGCCTGAAGGGTTTCAGGTGGTTAAAGTGGGGGCC
AATGGAGAGGTGGAGACACTAGAACAAGGGGAACTTCCACCCCAG
GAAGATCCTAGTTGGCAAAAAGACCCAGACTATCAGCCACCAGCC
AAAAAAACAAAGAAAACCAAAAAGAGC
98.
ZIM3(KRAB)-MAX-CTCF peptide
SEQ ID NO: 98
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGVVNMEEQPINIGELQLVQVPVPVTVPVATTSVEELQGAYENE
VSKEGLAESEPMICHTLPLPEGFQVVKVGANGEVETLEQGELPPQ
EDPSWQKDPDYQPPAKKTKKTKKS
99.
KOX1(KRAB)-MeCP2(t)-SCMH1 DNA
SEQ ID NO: 99
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTGGGCAGCGCTTCTGCTGGATCCCCAGGGTCGGACCGA
TACCTGGAGAGCCGCGATGCCTCTCGACTGAGTGGCCGGGACCCC
TCCTCATGGACAGTCGAGGATGTGATGCAGTTTGTCCGGGAAGCT
GATCCTCAGCTTGGACCCCACGCTGACCTGTTTCGCAAACACGAG
ATCGATGGCAAGGCCCTGCTGCTGCTGCGCAGTGACATGATGATG
AAGTACATGGGCCTGAAGCTGGGGCCTGCACTCAAGCTCTCCTAC
CACATTGACCGGCTGAAGCAGGGCAAGTTC
100.
KOX1(KRAB)-MeCP2(t)-SCMH1 peptide
SEQ ID NO: 100
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEVGSASAGSPGSDRYLESRDASRLSGRDP
SSWTVEDVMQFVREADPQLGPHADLFRKHEIDGKALLLLRSDMMM
KYMGLKLGPALKLSYHIDRLKQGKF
101.
ZIM3(KRAB)-MeCP2-KOX(KRAB) DNA
SEQ ID NO: 101
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGAAGCCTCAGTGCAGGTGAAAAGGGTGCTGGAAAAA
TCCCCCGGCAAACTCCTCGTGAAGATGCCCTTCCAGGCTTCCCCT
GGCGGAAAAGGTGAAGGGGGTGGCGCAACCACATCTGCCCAGGTC
ATGGTCATCAAGCGACCTGGAAGGAAAAGAAAGGCCGAGGCTGAC
CCTCAGGCCATTCCAAAGAAACGGGGACGCAAGCCAGGGTCCGTG
GTCGCAGCTGCAGCAGCTGAGGCTAAGAAAAAGGCAGTGAAGGAA
AGCTCCATCCGCAGTGTGCAGGAGACTGTCCTGCCCATCAAGAAG
AGGAAGACTAGGGAGACCGTGTCCATCGAGGTCAAAGAAGTGGTC
AAGCCCCTGCTCGTGTCCACCCTGGGCGAAAAATCTGGAAAGGGG
CTCAAAACATGCAAGTCACCTGGACGGAAAAGCAAGGAGTCTAGT
CCAAAGGGGCGCTCAAGCTCCGCTTCTAGTCCCCCTAAAAAGGAA
CACCATCACCATCACCATCACGCCGAGTCTCCTAAGGCTCCTATG
CCACTGCTCCCACCACCTCCACCACCTGAGCCACAGTCAAGCGAA
GACCCCATCAGCCCACCCGAGCCTCAGGATCTGTCCTCTAGTATT
TGCAAAGAGGAAAAGATGCCCAGAGCAGGCAGCCTGGAGAGTGAT
GGCTGTCCAAAAGAACCCGCCAAGACCCAGCCTATGGTGGCAGCC
GCTGCAACTACCACCACAACCACAACTACCACAGTGGCCGAAAAA
TACAAGCATCGCGGCGAGGGCGAACGAAAGGACATTGTGTCAAGC
TCCATGCCCAGACCTAACCGGGAGGAACCAGTCGATAGTAGGACA
CCCGTGACTGAGAGAGTCTCAGGCAGCGCTTCTGCTGGACGGACA
CTGGTGACCTTCAAGGATGTGTTTGTGGACTTCACCAGGGAGGAG
TGGAAGCTGCTGGACACTGCTCAGCAGATCCTGTACAGAAATGTG
ATGCTGGAGAACTATAAGAACCTGGTTTCCTTGGGTTATCAGCTT
ACTAAGCCAGATGTGATCCTCCGGTTGGAGAAGGGAGAAGAGCCC
TGGCTGGTG
102.
ZIM3(KRAB)-MeCP2-KOX(KRAB) peptide
SEQ ID NO: 102
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGEASVQVKRVLEKSPGKLLVKMPFQASP
GGKGEGGGATTSAQVMVIKRPGRKRKAEADPQAIPKKRGRKPGSV
VAAAAAEAKKKAVKESSIRSVQETVLPIKKRKTRETVSIEVKEVV
KPLLVSTLGEKSGKGLKTCKSPGRKSKESSPKGRSSSASSPPKKE
HHHHHHHAESPKAPMPLLPPPPPPEPQSSEDPISPPEPQDLSSSI
CKEEKMPRAGSLESDGCPKEPAKTQPMVAAAATTTTTTTTTVAEK
YKHRGEGERKDIVSSSMPRPNREEPVDSRTPVTERVSGSASAGRT
LVTFKDVFVDFTREEWKLLDTAQQILYRNVMLENYKNLVSLGYQL
TKPDVILRLEKGEEPWLV
103.
KOX1(KRAB)-MeCP2(t)-RYBP DNA
SEQ ID NO: 103
CGGACACTGGTGACCTTCAAGGATGTGTTTGTGGACTTCACCAGG
GAGGAGTGGAAGCTGCTGGACACTGCTCAGCAGATCCTGTACAGA
AATGTGATGCTGGAGAACTATAAGAACCTGGTTTCCTTGGGTTAT
CAGCTTACTAAGCCAGATGTGATCCTCCGGTTGGAGAAGGGAGAA
GAGCCCTGGCTGGTGGGCTCTGGCAGCGCTTCTGCTGGAACCACA
TCCACCCAGGTCATGGTGATCAAACGCCCCGGCAGGAAGCGAAAA
GCTGAGGCCGACCCTCAGGCCATTCCCAAGAAACGGGGCCGAAAG
CCGGGGAGTGTGGTGGCAGCCGCTGCCGCCGAGGCCAAAAAGAAA
GCCGTGAAGGAGTCTTCTATCCGATCTGTGCAGGAGACCGTACTC
CCCATCAAGAAGCGCAAGACCCGGGAGACCGTCAGCATCGAGGTC
AAGGAAGTGGGCAGCGCTTCTGCTGGAGATCCTCCTAGTGAAGCA
AACAGCATACAGTCTGCAAATGCTACAACAAAGACCAGCGAAACA
AATCACACCTCAAGGCCCCGGCTGAAAAACGTGGACAGGAGCACT
GCACAGCAGTTGGCAGTAACTGTGGGCAACGTCACCGTCATTATC
ACAGACTTTAAGGAAAAGACTCGCTCCTCATCGACATCCTCATCC
ACAGTGACCTCCAGTGCAGGGTCAGAACAGCAGAACCAGAGCAGC
TCGGGGTCAGAGAGCACAGACAAGGGCTCCTCCCGTTCCTCCACG
CCAAAGGGCGACATG
104.
KOX1(KRAB)-MeCP2(t)-RYBP peptide
SEQ ID NO: 104
RTLVTFKDVFVDFTREEWKLLDTAQQILYRNVMLENYKNLVSLGY
QLTKPDVILRLEKGEEPWLVGSGSASAGTTSTQVMVIKRPGRKRK
AEADPQAIPKKRGRKPGSVVAAAAAEAKKKAVKESSIRSVQETVL
PIKKRKTRETVSIEVKEVGSASAGDPPSEANSIQSANATTKTSET
NHTSRPRLKNVDRSTAQQLAVTVGNVTVIITDFKEKTRSSSTSSS
TVTSSAGSEQQNQSSSGSESTDKGSSRSSTPKGDM
105.
KRBOX1(KRAB)-MAX-MGA DNA
SEQ ID NO: 105
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGACAGCCGTCCTGTACTCACATC
TCTGCAGATGAAAAAGCAGCTGAAAGGAGTCGAAAGGCTCCACCA
ATTCCTCTAAAACTGAAGCCTGATTACTGGAGTGACAAACTACAG
AAAGAAGCAGAAGCGTTTGCTTATTATCGCCGGACACACACTGCC
AATGAGCGGCGGCGGCGTGGTGAAATGAGGGATCTCTTTGAGAAA
TTAAAGATCACATTGGGATTACTTCATTCTTCCAAGGTTTCCAAA
AGTCTCATTCTTACTCGAGCCTTCAGTGAAATTCAGGGACTAACA
GATCAGGCAGACAAATTGATAGGACAGAAAAATCTCCTGACTCGA
AAACGGAATATTCTGATACGGAAAGTA
106.
KRBOX1(KRAB)-MAX-MGA peptide
SEQ ID NO: 106
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGQPSCTHI
SADEKAAERSRKAPPIPLKLKPDYWSDKLQKEAEAFAYYRRTHTA
NERRRRGEMRDLFEKLKITLGLLHSSKVSKSLILTRAFSEIQGLT
DQADKLIGQKNLLTRKRNILIRKV
107.
KRBOX1(KRAB)-MAX-ZNF264(KRAB) DNA
SEQ ID NO: 107
ATGACAGCTGTGTCCTTAACAACCAGGCCCCAGGAATCAGTGGCT
TTTGAGGACGTGGCTGTGTACTTCACTACGAAGGAATGGGCCATC
ATGGTGCCTGCCGAGAGGGCCTTGTACAGGGATGTGATGCTGGAG
AACTATGAGGCTGTGGCCTTTGTAGTGCCACCCACTTCCAAACCA
GCTTTGGTCTCTCATCTGGAGCAAGGGAAAGAGTCCTGTTTCACC
CAGCCACAGGGAGTCCTAAGCAGGAATGACTGGAGAGCAGGCTGG
GGCTCTGGCAGCGCTTCTGCTGGAAGCGATAACGATGACATCGAG
GTGGAGAGCGACGAAGAGCAACCGAGGTTTCAATCTGCGGCTGAC
AAACGGGCTCATCATAATGCACTGGAACGAAAACGTAGGGACCAC
ATCAAAGACAGCTTTCACAGTTTGCGGGACTCAGTCCCATCACTC
CAAGGAGAGAAGGCATCCCGGGCCCAAATCCTAGACAAAGCCACA
GAATATATCCAGTATATGCGAAGGAAAAACCACACACACCAGCAA
GATATTGACGACCTCAAGCGGCAGAATGCTCTTCTGGAGCAGCAA
GTCCGTGCACTGGAGAAGGCGAGGTCAAGTGCCCAACTGCAGACC
AACTACCCCTCCTCAGACAACAGCCTCTACACCAACGCCAAGGGC
AGCACCATCTCTGCCTTCGATGGGGGCTCGGACTCCAGCTCGGAG
TCTGAGCCTGAAGAGCCCCAAAGCAGGAAGAAGCTCCGGATGGAG
GCCAGCGGCAGCGCTTCTGCTGGAGCGGCAGCGGTGCTGACGGAC
CGGGCCCAGGTGTCTGTGACCTTTGATGATGTGGCTGTGACTTTC
ACCAAGGAGGAGTGGGGGCAGCTGGACCTAGCTCAGCGGACCCTG
TACCAGGAGGTGATGCTGGAAAACTGTGGGCTCCTGGTGTCTCTG
GGGTGTCCTGTTCCCAAAGCTGAGCTGATCTGCCACCTAGAGCAT
GGGCAGGAGCCATGGACCAGGAAGGAAGACCTCTCCCAAGACACC
TGTCCAGGCGACAAAGGAAAACCTAAGACCACAGAACCTACCACT
TGTGAGCCAGCCTTGTCAGAG
108.
KRBOX1(KRAB)-MAX-ZNF264(KRAB) peptide
SEQ ID NO: 108
MTAVSLTTRPQESVAFEDVAVYFTTKEWAIMVPAERALYRDVMLE
NYEAVAFVVPPTSKPALVSHLEQGKESCFTQPQGVLSRNDWRAGW
GSGSASAGSDNDDIEVESDEEQPRFQSAADKRAHHNALERKRRDH
IKDSFHSLRDSVPSLQGEKASRAQILDKATEYIQYMRRKNHTHQQ
DIDDLKRQNALLEQQVRALEKARSSAQLQTNYPSSDNSLYTNAKG
STISAFDGGSDSSSESEPEEPQSRKKLRMEASGSASAGAAAVLTD
RAQVSVTFDDVAVTFTKEEWGQLDLAQRTLYQEVMLENCGLLVSL
GCPVPKAELICHLEHGQEPWTRKEDLSQDTCPGDKGKPKTTEPTT
CEPALSE
109.
ZIM3(KRAB)-MAX-ZIM3(KRAB) DNA
SEQ ID NO: 109
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAGCGATAACGATGACATCGAGGTGGAGAGCGACGAA
GAGCAACCGAGGTTTCAATCTGCGGCTGACAAACGGGCTCATCAT
AATGCACTGGAACGAAAACGTAGGGACCACATCAAAGACAGCTTT
CACAGTTTGCGGGACTCAGTCCCATCACTCCAAGGAGAGAAGGCA
TCCCGGGCCCAAATCCTAGACAAAGCCACAGAATATATCCAGTAT
ATGCGAAGGAAAAACCACACACACCAGCAAGATATTGACGACCTC
AAGCGGCAGAATGCTCTTCTGGAGCAGCAAGTCCGTGCACTGGAG
AAGGCGAGGTCAAGTGCCCAACTGCAGACCAACTACCCCTCCTCA
GACAACAGCCTCTACACCAACGCCAAGGGCAGCACCATCTCTGCC
TTCGATGGGGGCTCGGACTCCAGCTCGGAGTCTGAGCCTGAAGAG
CCCCAAAGCAGGAAGAAGCTCCGGATGGAGGCCAGCGGCAGCGCT
TCTGCTGGAATGAACAATTCCCAGGGAAGAGTGACCTTCGAGGAT
GTCACTGTGAACTTCACCCAGGGGGAGTGGCAGCGGCTGAATCCC
GAACAGAGAAACTTGTACAGGGATGTGATGCTGGAGAATTACAGC
AACCTTGTCTCTGTGGGACAAGGGGAAACCACCAAACCCGATGTG
ATCTTGAGGTTGGAACAAGGAAAGGAGCCGTGGTTGGAGGAAGAG
GAAGTGCTGGGAAGTGGCCGTGCAGAAAAAAATGGGGACATTGGA
GGGCAGATTTGGAAGCCAAAGGATGTGAAAGAGAGTCTC
110.
ZIM3(KRAB)-MAX-ZIM3(KRAB) peptide
SEQ ID NO: 110
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGSDNDDIEVESDEEQPRFQSAADKRAHH
NALERKRRDHIKDSFHSLRDSVPSLQGEKASRAQILDKATEYIQY
MRRKNHTHQQDIDDLKRQNALLEQQVRALEKARSSAQLQTNYPSS
DNSLYTNAKGSTISAFDGGSDSSSESEPEEPQSRKKLRMEASGSA
SAGMNNSQGRVTFEDVTVNFTQGEWQRLNPEQRNLYRDVMLENYS
NLVSVGQGETTKPDVILRLEQGKEPWLEEEEVLGSGRAEKNGDIG
GQIWKPKDVKESL
111.
MeCP2(NID) DNA
SEQ ID NO: 111
GCCAAAAAGAAAGCCGTGAAGGAGTCTTCTATCCGATCTGTGCAG
GAGACCGTACTCCCCATCAAGAAGCGCAAGACCCGGGAGACCGTC
AGCATCGAGGTCAAGGAAGTG
112.
MeCP2(NID) peptide
SEQ ID NO: 112
AKKKAVKESSIRSVQETVLPIKKRKTRETVSIEVKEV
113.
ZIM3(KRAB)-MeCP2(NID) DNA
SEQ ID NO: 113
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAGCCAAAAAGAAAGCCGTGAAGGAGTCTTCTATCCGA
TCTGTGCAGGAGACCGTACTCCCCATCAAGAAGCGCAAGACCCGG
GAGACCGTCAGCATCGAGGTCAAGGAAGTG
114.
ZIM3(KRAB)-MeCP2(NID) peptide
SEQ ID NO: 114
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGAKKKAVKESSIRSVQETVLPIKKRKTR
ETVSIEVKEV
115.
IPO8 Forward Primer
SEQ ID NO: 115
GGCATACAGTTTAACCTGCCAC
116.
IPO8 Reverse Primer
SEQ ID NO: 116
CAGGAGAGGCATCATGTCTGTAA
117.
CANX Forward Primer
SEQ ID NO: 117
GATCCAGACGCAGAGAAACC
118.
CANX Reverse Primer
SEQ ID NO: 118
CATCCAGGAGCTGACTCACA
119.
SYVN1 Forward Primer
SEQ ID NO: 119
ACCAGCATCCCTAGCTCAGA
120.
SYVN1 Reverse Primer
SEQ ID NO: 120
TCCTCAGGCATCTCCTCTGT
121.
BLM Forward Primer
SEQ ID NO: 121
CAGACTCCGAAGGAAGTTGTATG
122.
BLM Reverse Primer
SEQ ID NO: 122
TTTGGGGTGGTGTAACAAATGAT
123.
SEL1L Forward Primer
SEQ ID NO: 123
GAGGGGGAAAGTGTCACAGA
124.
SELIL Reverse Primer
SEQ ID NO: 124
GGTCAAAGCTGGTTTCCGTA
125.
DNAJC19 Forward Primer
SEQ ID NO: 125
AGTGGTAGCAGTTGGACTGAC
126.
DNAJC19 Reverse Primer
SEQ ID NO: 126
GGCAGATTTTGGTAGGCTTTGAA
127.
KRAS Forward Primer
SEQ ID NO: 127
ACAGAGAGTGGAGGATGCTTT
128.
KRAS Reverse Primer
SEQ ID NO: 128
TTTCACACAGCCAGGAGTCTT
129.
MRPS11 Forward Primer
SEQ ID NO: 129
CATCCGAGTTGTGGTGAAAGG
130.
MRPS11 Reverse Primer
SEQ ID NO: 130
GATTGGGGTGTTGTCTGTGATT
131.
sgRNA scaffold
SEQ ID NO: 131
GTTTCAGAGCTACAGCAGAAATGCTGTAGCAAGTTGAAATAAGGC
TAGTCCGTTATCAACTTGAAAAAGTGGCACCGAGTCGGTGCTTTT
T
SEQ ID NO: 132
sgRNA scaffold with pp7 stem loop132.
GTTTAAGAGCTATGCTGCGAATACGAGGGAGCAGACGATATGGCG
TCGCTCCTCTCCACGAGAGCATATGGGCTCCGTGGTCTCGTACAC
CATCAGGGTACGTATCAGACACCATCAGGGTCTGCTCGTATTCGC
AGCATAGCAAGTTTAAATAAGGCTAGTCCGTTATCAACTTGAAAA
AGTGGCACCGAGTCGGTGC
133.
Non-targeting spacer sequence
SEQ ID NO: 133
GAAATGTGAGATCAGAGTAAT
134.
SV40-eGFP Reporter −229/301(T) spacer sequence
SEQ ID NO: 134
GAAAGTCCCCAGGCTCCCCAGC
135.
SV40−eGFP Reporter −43(NT) spacer sequence
SEQ ID NO: 135
CTACTTCTGGAATAGCTCAG
136.
SV40−eGFP Reporter −68(NT) spacer sequence
SEQ ID NO: 136
CTATTCCAGAAGTAGTGAGG
137.
SV40−eGFP Reporter −134(NT) spacer sequence
SEQ ID NO: 137
GCCATGGGGCGGAGAATGGG
138.
SV40−eGFP Reporter −258(T) spacer sequence
SEQ ID NO: 138
ATCTCAATTAGTCAGCAACC
139.
SV40−eGFP Reporter −349(NT) spacer sequence
SEQ ID NO: 139
CTAACTGACACACTCTAGAG
140.
SV40−eGFP Reporter −313(T) spacer sequence
SEQ ID NO: 140
TTAGGGTGTGGAAAGTCCCC
141.
SV40−eGFP Reporter +153(T) spacer sequence
SEQ ID NO: 141
CTGAAGTTCATCTGCACCAC
142.
SV40−eGFP Reporter +31(T) spacer sequence
SEQ ID NO: 142
GGGCGAGGAGCTGTTCACCG
143.
CANX spacer sequence
SEQ ID NO: 143
TCGGGCCTGTGAGGACCTCG
144.
SYVN spacer sequence
SEQ ID NO: 144
ACACCTCACTTCCGGCGGCG
145.
BLM spacer sequence
SEQ ID NO: 145
AGGAAACGGAAGAACCCGAG
146.
SELIL spacer sequence
SEQ ID NO: 146
ATACTGACCCGAGGACGCCG
147.
DNAJC19 sgRNA A spacer sequence
SEQ ID NO: 147
GGGATGAGCCGTGCTCCCGG
148.
DNAJC19 sgRNA B spacer sequence
SEQ ID NO: 148
GGGCGCCTGTGCTTGAGGTT
149.
DNAJC19 sgRNA C spacer sequence
SEQ ID NO: 149
GGTGCTGTGAAGATGTGTTA
150.
KRAS sgRNA A spacer sequence
SEQ ID NO: 150
GCGGCGAAGGTGGCGGCGGCT
151.
KRAS sgRNA B spacer sequence
SEQ ID NO: 151
GGCAGTGGCGGCGGCGAAGG
152.
KRAS sgRNA C spacer sequence
SEQ ID NO: 152
GCTCCCAGTCCGAAATGGCG
153.
MRPS11 sgRNA A spacer sequence
SEQ ID NO: 153
GCTGCAGACGGAAACTGACT
154.
MRPS11 sgRNA B spacer sequence
SEQ ID NO: 154
GGGGTCAATTCAAGTCATGC
155.
MRPS11 sgRNA C spacer sequence
SEQ ID NO: 155
GTGGCTCAAGGGACACGAGT
156.
MeCP2(NLS1X) DNA
SEQ ID NO: 156
ACCACATCCACCCAGGTCATGGTGATCAAACGCCCCGGCAGGAAG
CGAAAAGCTGAGGCCGACCCTCAGGCCATTCCCAAGAAACGGGGC
CGAAAGCCGGGGAGTGTG
157.
MeCP2(NLS1X) peptide
SEQ ID NO: 157
TTSTQVMVIKRPGRKRKAEADPQAIPKKRGRKPGSV
158.
ZIM3(KRAB)-MeCP2(NLS1X) DNA
SEQ ID NO: 158
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAACCACATCCACCCAGGTCATGGTGATCAAACGCCCC
GGCAGGAAGCGAAAAGCTGAGGCCGACCCTCAGGCCATTCCCAAG
AAACGGGGCCGAAAGCCGGGGAGTGTG
159.
ZIM3(KRAB)-MeCP2(NLS1X) peptide
SEQ ID NO: 159
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGTTSTQVMVIKRPGRKRKAEADPQAIPK
KRGRKPGSV
160.
MeCP2(NLS2X) DNA
SEQ ID NO: 160
ACCACATCCACCCAGGTCATGGTGATCAAACGCCCCGGCAGGAAG
CGAAAAGCTGAGGCCGACCCTCAGGCCATTCCCAAGAAACGGGGC
CGAAAGCCGGGGAGTGTGGGATCTGGGAAATCTGGGTCAGGAACC
ACATCCACCCAGGTCATGGTGATCAAACGCCCCGGCAGGAAGCGA
AAAGCTGAGGCCGACCCTCAGGCCATTCCCAAGAAACGGGGCCGA
AAGCCGGGGAGTGTG
161.
MeCP2(NLS2X) peptide
SEQ ID NO: 161
TTSTQVMVIKRPGRKRKAEADPQAIPKKRGRKPGSVGSGKSGSGT
TSTQVMVIKRPGRKRKAEADPQAIPKKRGRKPGSV
162.
ZIM3(KRAB)-MeCP2(NLS2X) DNA
SEQ ID NO: 162
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAACCACATCCACCCAGGTCATGGTGATCAAACGCCCC
GGCAGGAAGCGAAAAGCTGAGGCCGACCCTCAGGCCATTCCCAAG
AAACGGGGCCGAAAGCCGGGGAGTGTGGGATCTGGGAAATCTGGG
TCAGGAACCACATCCACCCAGGTCATGGTGATCAAACGCCCCGGC
AGGAAGCGAAAAGCTGAGGCCGACCCTCAGGCCATTCCCAAGAAA
CGGGGCCGAAAGCCGGGGAGTGTG
163.
ZIM3(KRAB)-MeCP2(NLS2X) peptide
SEQ ID NO: 163
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGTTSTQVMVIKRPGRKRKAEADPQAIPK
KRGRKPGSVGSGKSGSGTTSTQVMVIKRPGRKRKAEADPQAIPKK
RGRKPGSV
164.
SV40NLS DNA
SEQ ID NO: 164
CCGAAAAAGAAGCGTAAGGTT
165.
SEQ ID NO: 165
SV40NLS peptidePKKKRKV
166.
ZIM3(KRAB)-SV40NLS DNA
SEQ ID NO: 166
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGACCGAAAAAGAAGCGTAAGGTT
167.
ZIM3(KRAB)-SV40NLS peptide
SEQ ID NO: 167
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGPKKKRKV
168.
BPSV40NLS DNA
SEQ ID NO: 168
AAACGGACAGCCGACGGAAGCGAGTTCGAGTCACCAAAGAAGAAG
CGGAAAGTC
169.
BPSV40NLS peptide
SEQ ID NO: 169
KRTADGSEFESPKKKRKV
170.
ZIM3(KRAB)-BPSV40NLS DNA
SEQ ID NO: 170
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAAACGGACAGCCGACGGAAGCGAGTTCGAGTCACCA
AAGAAGAAGCGGAAAGTC
171.
ZIM3(KRAB)-BPSV40NLS peptide
SEQ ID NO: 171
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGKRTADGSEFESPKKKRKV
172.
vBPSV40NLS DNA
SEQ ID NO: 172
AAAAGAACCGCCGACGGCAGCGAGAAGCGCACCGCCGACAGCCAG
CACAGCACCCCCCCCAAGACCAAGCGCAAGGTG
173.
vBPSV40NLS peptide
SEQ ID NO: 173
KRTADGSEKRTADSQHSTPPKTKRKV
174.
ZIM3(KRAB)- vBPSV40NLS DNA
SEQ ID NO: 174
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAAAAGAACCGCCGACGGCAGCGAGAAGCGCACCGCC
GACAGCCAGCACAGCACCCCCCCCAAGACCAAGCGCAAGGTG
175.
ZIM3(KRAB)- vBPSV40NLS peptide
SEQ ID NO: 175
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGKRTADGSEKRTADSQHSTPPKTKRKV
176.
cMYCNLS DNA
SEQ ID NO: 176
CCCGCCGCCAAGCGCGTGAAGCTGGAC
177.
cMYCNLS peptide
SEQ ID NO: 177
PAAKRVKLD
178.
ZIM3(KRAB)-cMYCNLS DNA
SEQ ID NO: 178
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGACCCGCCGCCAAGCGCGTGAAGCTGGAC
179.
ZIM3(KRAB)- cMYCNLS peptide
SEQ ID NO: 179
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGPAAKRVKLD
180.
NucNLS DNA
SEQ ID NO: 180
AAACGCCCTGCCGCAACGAAGAAGGCTGGACAGGCGAAAAAGAAG
AAG
181.
NucNLS peptide
SEQ ID NO: 181
KRPAATKKAGQAKKKK
182.
ZIM3(KRAB)-NucNLS DNA
SEQ ID NO: 182
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAAACGCCCTGCCGCAACGAAGAAGGCTGGACAGGCG
AAAAAGAAGAAG
183.
ZIM3(KRAB)-NucNLS peptide
SEQ ID NO: 183
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGKRPAATKKAGQAKKKK
184.
vBPSV40-SV40NLS DNA
SEQ ID NO: 184
AAAAGAACCGCCGACGGCAGCGAGAAGCGCACCGCCGACAGCCAG
CACAGCACCCCCCCCAAGACCAAGCGCAAGGTGGGATCTGGGAAA
TCTGGGTCAGGACCGAAAAAGAAGCGTAAGGTT
185.
vBPSV40-SV40NLS peptide
SEQ ID NO: 185
KRTADGSEKRTADSQHSTPPKTKRKVGSGKSGSGPKKKRKV
186.
ZIM3(KRAB)-vBPSV40-SV40NLS DNA
SEQ ID NO: 186
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAAAAGAACCGCCGACGGCAGCGAGAAGCGCACCGCC
GACAGCCAGCACAGCACCCCCCCCAAGACCAAGCGCAAGGTGGGA
TCTGGGAAATCTGGGTCAGGACCGAAAAAGAAGCGTAAGGTT
187.
ZIM3(KRAB)-vBPSV40-SV40NLS peptide
SEQ ID NO: 187
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGKRTADGSEKRTADSQHSTPPKTKRKVG
SGKSGSGPKKKRKV
188.
vBPSV40NLS(2X) DNA
SEQ ID NO: 188
AAAAGAACCGCCGACGGCAGCGAGAAGCGCACCGCCGACAGCCAG
CACAGCACCCCCCCCAAGACCAAGCGCAAGGTGGGATCTGGGAAA
TCTGGGTCAGGAAAAAGAACCGCCGACGGCAGCGAGAAGCGCACC
GCCGACAGCCAGCACAGCACCCCCCCCAAGACCAAGCGCAAGGTG
189.
vBPSV40NLS(2X) peptide
SEQ ID NO: 189
KRTADGSEKRTADSQHSTPPKTKRKVGSGKSGSGKRTADGSEKRT
ADSQHSTPPKTKRKV
190.
ZIM3(KRAB)-vBPSV40NLS(2X) DNA
SEQ ID NO: 190
ATGAACAATTCCCAGGGAAGAGTGACCTTCGAGGATGTCACTGTG
AACTTCACCCAGGGGGAGTGGCAGCGGCTGAATCCCGAACAGAGA
AACTTGTACAGGGATGTGATGCTGGAGAATTACAGCAACCTTGTC
TCTGTGGGACAAGGGGAAACCACCAAACCCGATGTGATCTTGAGG
TTGGAACAAGGAAAGGAGCCGTGGTTGGAGGAAGAGGAAGTGCTG
GGAAGTGGCCGTGCAGAAAAAAATGGGGACATTGGAGGGCAGATT
TGGAAGCCAAAGGATGTGAAAGAGAGTCTCGGCTCTGGCAGCGCT
TCTGCTGGAAAAAGAACCGCCGACGGCAGCGAGAAGCGCACCGCC
GACAGCCAGCACAGCACCCCCCCCAAGACCAAGCGCAAGGTGGGA
TCTGGGAAATCTGGGTCAGGAAAAAGAACCGCCGACGGCAGCGAG
AAGCGCACCGCCGACAGCCAGCACAGCACCCCCCCCAAGACCAAG
CGCAAGGTG
191.
ZIM3(KRAB)-vBPSV40NLS(2X) peptide
SEQ ID NO: 191
MNNSQGRVTFEDVTVNFTQGEWQRLNPEQRNLYRDVMLENYSNLV
SVGQGETTKPDVILRLEQGKEPWLEEEEVLGSGRAEKNGDIGGQI
WKPKDVKESLGSGSASAGKRTADGSEKRTADSQHSTPPKTKRKVG
SGKSGSGKRTADGSEKRTADSQHSTPPKTKRKV

What is claimed is:

1. A CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

2. The CRISPRi system of claim 1, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

3. The CRISPRi system of claim 1, wherein the two or more repressor domains comprise SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, or a fragment thereof.

4. The CRISPRi system of claim 1, wherein the three or more repressor domains comprise SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 114, or a fragment thereof.

5. The CRISPRi system of claim 1, wherein the catalytically inactive nuclease comprises a dCas nuclease selected from a dCas9, dCas12a, and dCas13.

6. The CRISPRi system of claim 1, wherein the catalytically inactive nuclease comprises at least 90% sequence identity to SEQ ID NO: 2.

7. The CRISPRi system of claim 1, wherein the repressor fusion peptide is fused to a nuclear localization signal.

8. An expression vector comprising one or more nucleic acids encoding a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

9. The expression vector of claim 8, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

10. The expression vector of claim 8, wherein the one or more nucleic acids encodes the two or more repressor domains comprising SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, or a fragment thereof.

11. The expression vector of claim 8, wherein the one or more nucleic acids encodes the three or more repressor domains comprising SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO:89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 113, or a fragment thereof.

12. The expression vector of claim 8, wherein the one or more nucleic acid encoding the catalytically inactive nuclease comprises at least 90% sequence identity to SEQ ID NO: 1.

13. The expression vector of claim 8, wherein the one or more nucleic acids encodes the two or more repressor fusion peptides fused to a nuclear localization signal.

14. An engineered cell comprising a CRISPR interference (CRISPRi) system, wherein the CRISPRi system comprises a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

15. The cell of claim 14, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.

16. The cell of claim 14, wherein the two or more repressor domains comprise SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, or a fragment thereof.

17. The cell of claim 14, wherein the three or more repressor domains comprise SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 114, or a fragment thereof.

18. The cell of claim 14, wherein the catalytically inactive nuclease comprises at least 90% sequence identity to SEQ ID NO: 2.

19. The cell of claim 14, wherein the repressor fusion peptide is fused to a nuclear localization signal.

20. A method of decreasing gene expression, the method comprising administering a CRISPR interference (CRISPRi) system comprising a single guide RNA (sgRNA) and a catalytically inactive nuclease operably fused to a repressor fusion peptide, wherein the repressor fusion peptide comprises two or more repressor domains comprising KOX1, KRBOX1, ZIM3, or a fragment thereof, fused to MAX, MeCP2, MeCP2(t), TRIM28, RYBP, CBX1, SCMH1, CTCF, REST, MGA, KLF10, IRF2BP1, IKZF5, RCOR1, ZNF554, ZNF264, or a fragment thereof.

21. The method of claim 20, wherein the repressor fusion peptide comprises three or more repressor domains comprising any combination of KOX1, KRBOX1, ZIM3, MAX, MeCP2t, MeCP2, or a fragment thereof, fused to TRIM28, RYBP, CBX1, SCMH1, CTCF, REST, MGA, KLF10, IRF2BP1, IKZF5, RCOR1, ZNF554, ZNF264, or a fragment thereof.


SEQUENCE LISTING (191 sequences)

SEQ ID NO: 1 - DNA, 4104 bp, synthetic construct
  misc_feature 1..4104: note = Description of sequence: dCas9
  source 1..4104: mol_type = other DNA
atggacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggtgat

SEQ ID NO: 2 - protein, 1368 aa, synthetic construct
  REGION 1..1368: note = Description of sequence: dCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - DNA, 30 bp, synthetic construct
  misc_feature 1..30: note = Description of sequence: Linkers Employed in dCas9-repressor fusion constructs, Between dCas9 and Repressor Domain 1
  source 1..30: mol_type = other DNA
ggctccggaagtgggtctagaggtggagcc

SEQ ID NO: 4 - protein, 10 aa, synthetic construct
  REGION 1..10: note = Description of sequence: Linkers Employed in dCas9-repressor fusion constructs, Between dCas9 and Repressor Domain 1
GSGSGSRGGA

SEQ ID NO: 5 - DNA, 24 bp, synthetic construct
  misc_feature 1..24: note = Description of sequence: Linkers Employed in dCas9-repressor fusion constructs, Between Repressor Domain 1 and Repressor Domain 2 (in bipartite fusions and tripartite fusions)
  source 1..24: mol_type = other DNA
ggctctggcagcgcttctgctgga

SEQ ID NO: 6 - protein, 8 aa, synthetic construct
  REGION 1..8: note = Description of sequence: Linkers Employed in dCas9-repressor fusion constructs,Between Repressor Domain 1 and Repressor Domain 2 (in bipartite fusions and tripartite fusions)
GSGSASAG

SEQ ID NO: 7 - DNA, 18 bp, synthetic construct
  misc_feature 1..18: note = Description of sequence: Linkers Employed in dCas9-repressor fusion constructs, Between Repressor Domain 1 and Repressor Domain 2 (in tripartite fusions)
  source 1..18: mol_type = other DNA
ggcagcgcttctgctgga

SEQ ID NO: 8 - protein, 6 aa, synthetic construct
  REGION 1..6: note = Description of sequence: Linkers Employed in dCas9-repressor fusion constructs, Between Repressor Domain 1 and Repressor Domain 2 (in tripartite fusions)
GSASAG

SEQ ID NO: 9 - DNA, 270 bp, synthetic construct
  misc_feature 1..270: note = Description of sequence: Repressor Domains, KRBOX1
  source 1..270: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctgg

SEQ ID NO: 10 - protein, 90 aa, synthetic construct
  REGION 1..90: note = Description of sequence: Repressor Domains, KRBOX1
MTAVSLTTRPQESVAFEDVAVYFTTKEWAIMVPAERALYRDVMLENYEAVAFVVPPTSKPALVSHLEQGKESCFTQPQGVLSRNDWRAGW

SEQ ID NO: 11 - DNA, 945 bp, synthetic construct
  misc_feature 1..945: note = Description of sequence: Repressor Domains, TRIM28
  source 1..945: mol_type = other DNA
cactgcggcgtgtgcagagagcgcctgcgacccgagagggagccccgcctgctgccctgtttgcactcggcctgtagtgcctgcttagggcccgcggcccccgccgccgccaacagctcgggggacggcggggcggcgggcgacggcaccgtggtggactgtcccgtgtgcaagcaacagtgcttctccaaagacatcgtggagaattatttcatgcgtgatagtggcagcaaggctgccaccgacgcccaggatgcgaaccagtgctgcactagctgtgaggataatgccccagccaccagctactgtgtggagtgctcggagcctctgtgtgagacctgtgtagaggcgcaccagcgggtgaagtacaccaaggaccatactgtgcgctctactgggccagccaagtctcgggatggtgaacgtactgtctattgcaacgtacacaagcatgaaccccttgtgctgttttgtgagagctgtgatactctcacctgccgagactgccagctcaatgcccacaaggaccaccagtaccagttcttagaggatgcagtgaggaaccagcgcaagctcctggcctcactggtgaagcgccttggggacaaacatgcaacattgcagaagagcaccaaggaggttcgcagctcaatccgccaggtgtctgacgtacagaagcgtgtgcaagtggatgtcaagatggccatcctgcagatcatgaaggagctgaataagcggggccgtgtgctggtcaatgatgcccagaaggtgactgaggggcagcaggagcgcctggagcggcagcactggaccatgaccaagatccagaagcaccaggagcacattctgcgctttgcctcttgggctctggagagtgacaacaacacagcccttttgctttctaagaagttgatctacttccagctgcaccgggccctcaagatg

SEQ ID NO: 12 - protein, 315 aa, synthetic construct
  REGION 1..315: note = Description of sequence: Repressor Domains, TRIM28
HCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKM

SEQ ID NO: 13 - DNA, 303 bp, synthetic construct
  misc_feature 1..303: note = Description of sequence: Repressor Domains, RYBP
  source 1..303: mol_type = other DNA
gatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatg

SEQ ID NO: 14 - protein, 101 aa, synthetic construct
  REGION 1..101: note = Description of sequence: Repressor Domains, RYBP
DPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDM

SEQ ID NO: 15 - DNA, 288 bp, synthetic construct
  misc_feature 1..288: note = Description of sequence: Repressor Domains, CBX1
  source 1..288: mol_type = other DNA
gattctgaagataagggagaggagagcaaaccaaagaagaagaaagaagagtcagaaaagccacgaggctttgctcgaggtttggagccggagcggattattggagctacagactccagtggagagctcatgttcctgatgaaatggaaaaactctgatgaggctgacctggtccctgccaaggaagccaatgtcaagtgcccacaggttgtcatatccttctatgaggaaaggctgacgtggcattcctacccctcggaggatgatgacaaaaaagatgacaagaac

SEQ ID NO: 16 - protein, 96 aa, synthetic construct
  REGION 1..96: note = Description of sequence: Repressor Domains, CBX1
DSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 17 - DNA, 240 bp, synthetic construct
  misc_feature 1..240: note = Description of sequence: Repressor Domains, MeCP2(t)
  source 1..240: mol_type = other DNA
accacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 18 - protein, 80 aa, synthetic construct
  REGION 1..80: note = Description of sequence: Repressor Domains, MeCP2(t)
TTSTQVMVIKRPGRKRKAEADPQAIPKKRGRKPGSVVAAAAAEAKKKAVKESSIRSVQETVLPIKKRKTRETVSIEVKEV

SEQ ID NO: 19 - DNA, 273 bp, synthetic construct
  misc_feature 1..273: note = Description of sequence: Repressor Domains, SCMH1
  source 1..273: mol_type = other DNA
tccccagggtcggaccgatacctggagagccgcgatgcctctcgactgagtggccgggacccctcctcatggacagtcgaggatgtgatgcagtttgtccgggaagctgatcctcagcttggaccccacgctgacctgtttcgcaaacacgagatcgatggcaaggccctgctgctgctgcgcagtgacatgatgatgaagtacatgggcctgaagctggggcctgcactcaagctctcctaccacattgaccggctgaagcagggcaagttc

SEQ ID NO: 20 - protein, 91 aa, synthetic construct
  REGION 1..91: note = Description of sequence: Repressor Domains, SCMH1
SPGSDRYLESRDASRLSGRDPSSWTVEDVMQFVREADPQLGPHADLFRKHEIDGKALLLLRSDMMMKYMGLKLGPALKLSYHIDRLKQGKF

SEQ ID NO: 21 - DNA, 333 bp, synthetic construct
  misc_feature 1..333: note = Description of sequence: Repressor Domains, CTCF
  source 1..333: mol_type = other DNA
gttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagc

SEQ ID NO: 22 - protein, 111 aa, synthetic construct
  REGION 1..111: note = Description of sequence: Repressor Domains, CTCF
VVNMEEQPINIGELQLVQVPVPVTVPVATTSVEELQGAYENEVSKEGLAESEPMICHTLPLPEGFQVVKVGANGEVETLEQGELPPQEDPSWQKDPDYQPPAKKTKKTKKS

SEQ ID NO: 23 - DNA, 243 bp, synthetic construct
  misc_feature 1..243: note = Description of sequence: Repressor Domains, REST
  source 1..243: mol_type = other DNA
ggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaa

SEQ ID NO: 24 - protein, 81 aa, synthetic construct
  REGION 1..81: note = Description of sequence: Repressor Domains, REST
GIHSHEGSDLSDNMSEGSDDSGLHGARPVPQESSRKNAKEALAVKAAKGDFVCIFCDRSFRKGKDYSKHLNRHLVNVYYLE

SEQ ID NO: 25 - DNA, 363 bp, synthetic construct
  misc_feature 1..363: note = Description of sequence: Repressor Domains, MGA
  source 1..363: mol_type = other DNA
cagccgtcctgtactcacatctctgcagatgaaaaagcagctgaaaggagtcgaaaggctccaccaattcctctaaaactgaagcctgattactggagtgacaaactacagaaagaagcagaagcgtttgcttattatcgccggacacacactgccaatgagcggcggcggcgtggtgaaatgagggatctctttgagaaattaaagatcacattgggattacttcattcttccaaggtttccaaaagtctcattcttactcgagccttcagtgaaattcagggactaacagatcaggcagacaaattgataggacagaaaaatctcctgactcgaaaacggaatattctgatacggaaagta

SEQ ID NO: 26 - protein, 121 aa, synthetic construct
  REGION 1..121: note = Description of sequence: Repressor Domains, MGA
QPSCTHISADEKAAERSRKAPPIPLKLKPDYWSDKLQKEAEAFAYYRRTHTANERRRRGEMRDLFEKLKITLGLLHSSKVSKSLILTRAFSEIQGLTDQADKLIGQKNLLTRKRNILIRKV

SEQ ID NO: 27 - DNA, 229 bp, synthetic construct
  misc_feature 1..229: note = Description of sequence: Repressor Domains, KLF10
  source 1..229: mol_type = other DNA
atggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattctttta

SEQ ID NO: 28 - protein, 135 aa, synthetic construct
  REGION 1..135: note = Description of sequence: Repressor Domains, KLF10
MAPAPSTVHFKSLSDTAKPHIAAPFKEEEKSPVSAPKLPKAQATSVIRHTADAQLCNHQTCPMKAASILNYQNNSFRRRTHLNVEAARKNIGAAGAAGAACCCACCTAAATGTTGAGGCTGCAAGAAAGAACATA

SEQ ID NO: 29 - DNA, 240 bp, synthetic construct
  misc_feature 1..240: note = Description of sequence: Repressor Domains, IRF2BP1
  source 1..240: mol_type = other DNA
gcgtctgtgcaggcgtcccgccgccagtggtgctacctgtgcgacctgcccaagatgccgtgggccatggtgtgggacttcagcgaggccgtgtgtcgcggctgcgtgaacttcgagggcgcggaccgcatcgaactgctcatcgatgccgcccgccagctcaagcgcagccacgtgctccccgagggccgctcgcccgggcccccggcccttaagcacccggccaccaaggacctggcg

SEQ ID NO: 30 - protein, 80 aa, synthetic construct
  REGION 1..80: note = Description of sequence: Repressor Domains, IRF2BP1
ASVQASRRQWCYLCDLPKMPWAMVWDFSEAVCRGCVNFEGADRIELLIDAARQLKRSHVLPEGRSPGPPALKHPATKDLA

SEQ ID NO: 31 - DNA, 477 bp, synthetic construct
  misc_feature 1..477: note = Description of sequence: Repressor Domains, MAX
  source 1..477: mol_type = other DNA
agcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagc

SEQ ID NO: 32 - protein, 159 aa, synthetic construct
  REGION 1..159: note = Description of sequence: Repressor Domains, MAX
SDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEAS

SEQ ID NO: 33 - DNA, 243 bp, synthetic construct
  misc_feature 1..243: note = Description of sequence: Repressor Domains, IKZF5
  source 1..243: mol_type = other DNA
agcactcccagcataggaaacagccagccaagcaccccagccccagccctgccggtccaggaccctcagcttctgcaccactgccagcactgtgatatgtactttgcagacaacatcctttacactattcatatgggatgtcatgggtatgaaaatccttttcagtgtaatatatgtggatgcaaatgtaaaaacaagtatgattttgcctgtcattttgcaagagggcaacataaccaacat

SEQ ID NO: 34 - protein, 81 aa, synthetic construct
  REGION 1..81: note = Description of sequence: Repressor Domains, IKZF5
STPSIGNSQPSTPAPALPVQDPQLLHHCQHCDMYFADNILYTIHMGCHGYENPFQCNICGCKCKNKYDFACHFARGQHNQH

SEQ ID NO: 35 - DNA, 918 bp, synthetic construct
  misc_feature 1..918: note = Description of sequence: Repressor Domains, RCOR1
  source 1..918: mol_type = other DNA
cccaatggcaacagcagcagcaactcctgggaggaaggcagctcgggctcgtccagcgacgaggagcacggtggcggtggcatgagggtcggaccccagtaccaggcggtggtgcccgacttcgaccccgccaaactggcaagacgcagtcaagaacgggacaatcttggcatgttggtctggtcacccaatcaaaatctgtcagaagcaaagttggatgaatacattgccattgccaaagaaaagcatgggtacaacatggaacaggctcttgggatgctcttctggcataaacataatatcgaaaagtcattggctgatttgcccaactttacccctttcccagatgagtggactgtggaagataaagtcttatttgagcaagcctttagttttcatgggaaaacttttcatagaatccaacaaatgcttccagataaatctatagcaagtctggtgaaattttactattcttggaagaagacgaggactaaaactagtgtgatggatcgccatgcccggaaacaaaaacgggagcgggaggagagcgaggatgaactggaagaggcaaatggaaacaatcccattgacattgaggttgatcaaaacaaggaaagcaaaaaggaggttccccctactgagacagttcctcaggtcaaaaaagaaaaacatagcacacaagctaaaaatagagcaaaaaggaaacctccaaaaggaatgtttctttctcaagaagatgtggaggctgtttctgccaatgccactgctgctaccacggtgctgagacaactagacatggaattggtttcagtcaaacgacagatccagaatattaaacagacaaacagtgctctcaaagaaaaacttgatggtggaatagaaccatatcgacttccagaggtcattcagaaatgtaat

SEQ ID NO: 36 - protein, 306 aa, synthetic construct
  REGION 1..306: note = Description of sequence: Repressor Domains, RCOR1
PNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCN

SEQ ID NO: 37 - DNA, 1191 bp, synthetic construct
  misc_feature 1..1191: note = Description of sequence: ZIM3(KRAB)-MeCP2
  source 1..1191: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctca

SEQ ID NO: 38 - protein, 397 aa, synthetic construct
  REGION 1..397: note = Description of sequence: ZIM3(KRAB)-MeCP2
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 39 - DNA, 459 bp, synthetic construct
  misc_feature 1..459: note = Description of sequence: KOX1(KRAB)-MeCP2(t)
  source 1..459: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 40 - protein, 153 aa, synthetic construct
  REGION 1..153: note = Description of sequence: KOX1(KRAB)-MeCP2(t)
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEV

SEQ ID NO: 41 - DNA, 801 bp, synthetic construct
  misc_feature 1..801: note = Description of sequence: ZIM3(KRAB)-MAX
  source 1..801: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagc

SEQ ID NO: 42 - protein, 267 aa, synthetic construct
  REGION 1..267: note = Description of sequence: ZIM3(KRAB)-MAX
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEAS

SEQ ID NO: 43 - DNA, 771 bp, synthetic construct
  misc_feature 1..771: note = Description of sequence: KRBOX1(KRAB)-MAX
  source 1..771: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagc

SEQ ID NO: 44 - protein, 257 aa, synthetic construct
  REGION 1..257: note = Description of sequence: KRBOX1(KRAB)-MAX
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEAS

SEQ ID NO: 45 - DNA, 696 bp, synthetic construct
  misc_feature 1..696: note = Description of sequence: KOX1(KRAB)-MAX
  source 1..696: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagc

SEQ ID NO: 46 - protein, 232 aa, synthetic construct
  REGION 1..232: note = Description of sequence: KOX1(KRAB)-MAX
RTLVTFKDVFVDFTREEWKLLDTAQQILYRNVMLENYKNLVSLGYQLTKPDVILRLEKGEEPWLVGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEAS

SEQ ID NO: 47 - DNA, 564 bp, synthetic construct
  misc_feature 1..564: note = Description of sequence: ZIM3(KRAB)-IRF2BP1
  source 1..564: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagcgtctgtgcaggcgtcccgccgccagtggtgctacctgtgcgacctgcccaagatgccgtgggccatggtgtgggacttcagcgaggccgtgtgtcgcggctgcgtgaacttcgagggcgcggaccgcatcgaactgctcatcgatgccgcccgccagctcaagcgcagccacgtgctccccgagggccgctcgcccgggcccccggcccttaagcacccggccaccaaggacctggcg

SEQ ID NO: 48 - protein, 188 aa, synthetic construct
  REGION 1..188: note = Description of sequence: ZIM3(KRAB)-IRF2BP1
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGASVQASRRQWCYLCDLPKMPWAMVWDFSEAVCRGCVNFEGADRIELLIDAARQLKRSHVLPEGRSPGPPALKHPATKDLA

SEQ ID NO: 49 - DNA, 624 bp, synthetic construct
  misc_feature 1..624: note = Description of sequence: ZIM3(KRAB)-ZIM3(KRAB)
  source 1..624: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctc

SEQ ID NO: 50 - protein, 208 aa, synthetic construct
  REGION 1..208: note = Description of sequence: ZIM3(KRAB)-ZIM3(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 51 - DNA, 627 bp, synthetic construct
  misc_feature 1..627: note = Description of sequence: KRBOX1(KRAB)-CTCF
  source 1..627: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggagttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagc

SEQ ID NO: 52 - protein, 209 aa, synthetic construct
  REGION 1..209: note = Description of sequence: KRBOX1(KRAB)-CTCF
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGVVNMEEQPINIGELQLVQVPVPVTVPVATTSVEELQGAYENEVSKEGLAESEPMICHTLPLPEGFQVVKVGANGEVETLEQGELPPQEDPSWQKDPDYQPPAKKTKKTKKS

SEQ ID NO: 53 - DNA, 753 bp, synthetic construct
  misc_feature 1..753: note = Description of sequence: ZIM3(KRAB)-ZNF554(KRAB)
  source 1..753: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggattttcccaagaggagagaatggctgctgggtacctgccccgctggtcccaggaattagtaacctttgaggacgtgtccatggacttctcccaggaggagtgggagttgctggagcctgctcagaagaacctgtacagagaggtgatgctggagaactacaggaacgtggtctccctggaagccttgaagaaccaatgtactgatgtggggattaaagagggtccactttccccagcacaaacctcacaagtcactagtctttcctcatggacggggtatttactttttcaaccagtggcttcttcccacttggagcaaagagaagccctgtggatagaggaaaaaggaactcctcaagcctcctgttcagattggatgactgtactaagaaaccaagactcaacttacaagaaggtggctttgcaggag

SEQ ID NO: 54 - protein, 251 aa, synthetic construct
  REGION 1..251: note = Description of sequence: ZIM3(KRAB)-ZNF554(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGFSQEERMAAGYLPRWSQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCSDWMTVLRNQDSTYKKVALQE

SEQ ID NO: 55 - DNA, 1161 bp, synthetic construct
  misc_feature 1..1161: note = Description of sequence: KRBOX1(KRAB)-MeCP2
  source 1..1161: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctca

SEQ ID NO: 56 - protein, 387 aa, synthetic construct
  REGION 1..387: note = Description of sequence: KRBOX1(KRAB)-MeCP2
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 57 - DNA, 627 bp, synthetic construct
  misc_feature 1..627: note = Description of sequence: ZIM3(KRAB)-RYBP
  source 1..627: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatg

SEQ ID NO: 58 - protein, 209 aa, synthetic construct
  REGION 1..209: note = Description of sequence: ZIM3(KRAB)-RYBP
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGDPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDM

SEQ ID NO: 59 - DNA, 597 bp, synthetic construct
  misc_feature 1..597: note = Description of sequence: ZIM3(KRAB)-KLF10
  source 1..597: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacata

SEQ ID NO: 60 - protein, 199 aa, synthetic construct
  REGION 1..199: note = Description of sequence: ZIM3(KRAB)-KLF10
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGMAPAPSTVHFKSLSDTAKPHIAAPFKEEEKSPVSAPKLPKAQATSVIRHTADAQLCNHQTCPMKAASILNYQNNSFRRRTHLNVEAARKNI

SEQ ID NO: 61 - DNA, 594 bp, synthetic construct
  misc_feature 1..594: note = Description of sequence: KRBOX1(KRAB)-ZIM3(KRAB)
  source 1..594: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctc

SEQ ID NO: 62 - protein, 198 aa, synthetic construct
  REGION 1..198: note = Description of sequence: KRBOX1(KRAB)-ZIM3(KRAB)
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 63 - DNA, 1059 bp, synthetic construct
  misc_feature 1..1059: note = Description of sequence: ZIM3(KRAB)-MAX-MeCP2(t)
  source 1..1059: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 64 - protein, 353 aa, synthetic construct
  REGION 1..353: note = Description of sequence: ZIM3(KRAB)-MAX-MeCP2(t)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGTTSTQVMVIKRPGRKRKAEADPQAIPKKRGRKPGSVVAAAAAEAKKKAVKESSIRSVQETVLPIKKRKTRETVSIEVKEV

SEQ ID NO: 65 - DNA, 717 bp, synthetic construct
  misc_feature 1..717: note = Description of sequence: KOX1(KRAB)-MeCP2(t)-MeCP2(t)
  source 1..717: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtgggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 66 - protein, 239 aa, synthetic construct
  REGION 1..239: note = Description of sequence: KOX1(KRAB)-MeCP2(t)-MeCP2(t)
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEVGSASAGTTSTQVMVIKRPGRKRKAEADPQAIPKKRGRKPGSVVAAAAAEAKKKAVKESSIRSVQETVLPIKKRKTRETVSIEVKEV

SEQ ID NO: 67 - DNA, 672 bp, synthetic construct
  misc_feature 1..672: note = Description of sequence: KOX1(KRAB)-MeCP2(t)-KOX1(KRAB)
  source 1..672: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtgggcagcgcttctgctggacggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 68 - protein, 224 aa, synthetic construct
  REGION 1..224: note = Description of sequence: KOX1(KRAB)-MeCP2(t)-KOX1(KRAB)
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEVGSASAGRTLVTFKDVFVDFTREEWKLLDTAQQILYRNVMLENYKNLVSLGYQLTKPDVILRLEKGEEPWLV

SEQ ID NO: 69 - DNA, 1059 bp, synthetic construct
  misc_feature 1..1059: note = Description of sequence: ZIM3(KRAB)-MAX-IRF2BP1
  source 1..1059: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggagcgtctgtgcaggcgtcccgccgccagtggtgctacctgtgcgacctgcccaagatgccgtgggccatggtgtgggacttcagcgaggccgtgtgtcgcggctgcgtgaacttcgagggcgcggaccgcatcgaactgctcatcgatgccgcccgccagctcaagcgcagccacgtgctccccgagggccgctcgcccgggcccccggcccttaagcacccggccaccaaggacctggcg

SEQ ID NO: 70 - protein, 353 aa, synthetic construct
  REGION 1..353: note = Description of sequence: ZIM3(KRAB)-MAX-IRF2BP1
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGASVQASRRQWCYLCDLPKMPWAMVWDFSEAVCRGCVNFEGADRIELLIDAARQLKRSHVLPEGRSPGPPALKHPATKDLA

SEQ ID NO: 71 - DNA, 789 bp, synthetic construct
  misc_feature 1..789: note = Description of sequence: KOX1(KRAB)-MeCP2(t)-ZNF264(KRAB)
  source 1..789: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtgggcagcgcttctgctggagcggcagcggtgctgacggaccgggcccaggtgtctgtgacctttgatgatgtggctgtgactttcaccaaggaggagtgggggcagctggacctagctcagcggaccctgtaccaggaggtgatgctggaaaactgtgggctcctggtgtctctggggtgtcctgttcccaaagctgagctgatctgccacctagagcatgggcaggagccatggaccaggaaggaagacctctcccaagacacctgtccaggcgacaaaggaaaacctaagaccacagaacctaccacttgtgagccagccttgtcagag

SEQ ID NO: 72 - protein, 263 aa, synthetic construct
  REGION 1..263: note = Description of sequence: KOX1(KRAB)-MeCP2(t)-ZNF264(KRAB)
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEVGSASAGAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSE

SEQ ID NO: 73 - DNA, 1029 bp, synthetic construct
  misc_feature 1..1029: note = Description of sequence: KRBOX1(KRAB)-MAX-MeCP2(t)
  source 1..1029: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 74 - protein, 343 aa, synthetic construct
  REGION 1..343: note = Description of sequence: KRBOX1(KRAB)-MAX-MeCP2(t)
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGTTSTQVMVIKRPGRKRKAEADPQAIPKKRGRKPGSVVAAAAAEAKKKAVKESSIRSVQETVLPIKKRKTRETVSIEVKEV

SEQ ID NO: 75 - DNA, 1512 bp, synthetic construct
  misc_feature 1..1512: note = Description of sequence: ZIM3(KRAB)-MeCP2-RYBP
  source 1..1512: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatg

SEQ ID NO: 76 - protein, 504 aa, synthetic construct
  REGION 1..504: note = Description of sequence: ZIM3(KRAB)-MeCP2-RYBP
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGDPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDM

SEQ ID NO: 77 - DNA, 1248 bp, synthetic construct
  misc_feature 1..1248: note = Description of sequence: ZIM3(KRAB)-MAX-ZNF554(KRAB)
  source 1..1248: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggattttcccaagaggagagaatggctgctgggtacctgccccgctggtcccaggaattagtaacctttgaggacgtgtccatggacttctcccaggaggagtgggagttgctggagcctgctcagaagaacctgtacagagaggtgatgctggagaactacaggaacgtggtctccctggaagccttgaagaaccaatgtactgatgtggggattaaagagggtccactttccccagcacaaacctcacaagtcactagtctttcctcatggacggggtatttactttttcaaccagtggcttcttcccacttggagcaaagagaagccctgtggatagaggaaaaaggaactcctcaagcctcctgttcagattggatgactgtactaagaaaccaagactcaacttacaagaaggtggctttgcaggag

SEQ ID NO: 78 - protein, 416 aa, synthetic construct
  REGION 1..416: note = Description of sequence: ZIM3(KRAB)-MAX-ZNF554(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGFSQEERMAAGYLPRWSQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCSDWMTVLRNQDSTYKKVALQE

SEQ ID NO: 79 - DNA, 1014 bp, synthetic construct
  misc_feature 1..1014: note = Description of sequence: ZIM3(KRAB)-MAX-KOX1(KRAB)
  source 1..1014: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggacggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 80 - protein, 338 aa, synthetic construct
  REGION 1..338: note = Description of sequence: ZIM3(KRAB)-MAX-KOX1(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGRTLVTFKDVFVDFTREEWKLLDTAQQILYRNVMLENYKNLVSLGYQLTKPDVILRLEKGEEPWLV

SEQ ID NO: 81 - DNA, 1479 bp, synthetic construct
  misc_feature 1..1479: note = Description of sequence: ZIM3(KRAB)-MeCP2-KRBOX1(KRAB)
  source 1..1479: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggaatgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctgg

SEQ ID NO: 82 - protein, 493 aa, synthetic construct
  REGION 1..493: note = Description of sequence: ZIM3(KRAB)-MeCP2-KRBOX1(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGMTAVSLTTRPQESVAFEDVAVYFTTKEWAIMVPAERALYRDVMLENYEAVAFVVPPTSKPALVSHLEQGKESCFTQPQGVLSRNDWRAGW

SEQ ID NO: 83 - DNA, 1089 bp, synthetic construct
  misc_feature 1..1089: note = Description of sequence: KRBOX1(KRAB)-MAX-ZIM3(KRAB)
  source 1..1089: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctc

SEQ ID NO: 84 - protein, 363 aa, synthetic construct
  REGION 1..363: note = Description of sequence: KRBOX1(KRAB)-MAX-ZIM3(KRAB)
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 85 - DNA, 1449 bp, synthetic construct
  misc_feature 1..1449: note = Description of sequence: ZIM3(KRAB)-MeCP2-MeCP2(t)
  source 1..1449: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtg

SEQ ID NO: 86 - protein, 483 aa, synthetic construct
  REGION 1..483: note = Description of sequence: ZIM3(KRAB)-MeCP2-MeCP2(t)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGTTSTQVMVIKRPGRKRKAEADPQAIPKKRGRKPGSVVAAAAAEAKKKAVKESSIRSVQETVLPIKKRKTRETVSIEVKEV

SEQ ID NO: 87 - DNA, 1131 bp, synthetic construct
  misc_feature 1..1131: note = Description of sequence: ZIM3(KRAB)-MAX-ZNF264(KRAB)
  source 1..1131: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggagcggcagcggtgctgacggaccgggcccaggtgtctgtgacctttgatgatgtggctgtgactttcaccaaggaggagtgggggcagctggacctagctcagcggaccctgtaccaggaggtgatgctggaaaactgtgggctcctggtgtctctggggtgtcctgttcccaaagctgagctgatctgccacctagagcatgggcaggagccatggaccaggaaggaagacctctcccaagacacctgtccaggcgacaaaggaaaacctaagaccacagaacctaccacttgtgagccagccttgtcagag

SEQ ID NO: 88 - protein, 377 aa, synthetic construct
  REGION 1..377: note = Description of sequence: ZIM3(KRAB)-MAX-ZNF264(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSE

SEQ ID NO: 89 - DNA, 1509 bp, synthetic construct
  misc_feature 1..1509: note = Description of sequence: ZIM3(KRAB)-MeCP2-ZIM3(KRAB)
  source 1..1509: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctc

SEQ ID NO: 90 - protein, 503 aa, synthetic construct
  REGION 1..503: note = Description of sequence: ZIM3(KRAB)-MeCP2-ZIM3(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 91 - DNA, 1656 bp, synthetic construct
  misc_feature 1..1656: note = Description of sequence: KRBOX1(KRAB)-MAX-MeCP2
  source 1..1656: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctca

SEQ ID NO: 92 - protein, 552 aa, synthetic construct
  REGION 1..552: note = Description of sequence: KRBOX1(KRAB)-MAX-MeCP2
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 93 - DNA, 1218 bp, synthetic construct
  misc_feature 1..1218: note = Description of sequence: KRBOX1(KRAB)-MAX-ZNF554(KRAB)
  source 1..1218: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggattttcccaagaggagagaatggctgctgggtacctgccccgctggtcccaggaattagtaacctttgaggacgtgtccatggacttctcccaggaggagtgggagttgctggagcctgctcagaagaacctgtacagagaggtgatgctggagaactacaggaacgtggtctccctggaagccttgaagaaccaatgtactgatgtggggattaaagagggtccactttccccagcacaaacctcacaagtcactagtctttcctcatggacggggtatttactttttcaaccagtggcttcttcccacttggagcaaagagaagccctgtggatagaggaaaaaggaactcctcaagcctcctgttcagattggatgactgtactaagaaaccaagactcaacttacaagaaggtggctttgcaggag

SEQ ID NO: 94 - protein, 406 aa, synthetic construct
  REGION 1..406: note = Description of sequence: KRBOX1(KRAB)-MAX-ZNF554(KRAB)
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGFSQEERMAAGYLPRWSQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCSDWMTVLRNQDSTYKKVALQE

SEQ ID NO: 95 - DNA, 1449 bp, synthetic construct
  misc_feature 1..1449: note = Description of sequence: ZIM3(KRAB)-MeCP2-IRFBP1
  source 1..1449: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggagcgtctgtgcaggcgtcccgccgccagtggtgctacctgtgcgacctgcccaagatgccgtgggccatggtgtgggacttcagcgaggccgtgtgtcgcggctgcgtgaacttcgagggcgcggaccgcatcgaactgctcatcgatgccgcccgccagctcaagcgcagccacgtgctccccgagggccgctcgcccgggcccccggcccttaagcacccggccaccaaggacctggcg

SEQ ID NO: 96 - protein, 483 aa, synthetic construct
  REGION 1..483: note = Description of sequence: ZIM3(KRAB)-MeCP2-IRFBP1
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGASVQASRRQWCYLCDLPKMPWAMVWDFSEAVCRGCVNFEGADRIELLIDAARQLKRSHVLPEGRSPGPPALKHPATKDLA

SEQ ID NO: 97 - DNA, 1152 bp, synthetic construct
  misc_feature 1..1152: note = Description of sequence: ZIM3(KRAB)-MAX-CTCF
  source 1..1152: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggagttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagc

SEQ ID NO: 98 - protein, 384 aa, synthetic construct
  REGION 1..384: note = Description of sequence: ZIM3(KRAB)-MAX-CTCF
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGVVNMEEQPINIGELQLVQVPVPVTVPVATTSVEELQGAYENEVSKEGLAESEPMICHTLPLPEGFQVVKVGANGEVETLEQGELPPQEDPSWQKDPDYQPPAKKTKKTKKS

SEQ ID NO: 99 - DNA, 750 bp, synthetic construct
  misc_feature 1..750: note = Description of sequence: KOX1(KRAB)-MeCP2(t)-SCMH1
  source 1..750: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtgggcagcgcttctgctggatccccagggtcggaccgatacctggagagccgcgatgcctctcgactgagtggccgggacccctcctcatggacagtcgaggatgtgatgcagtttgtccgggaagctgatcctcagcttggaccccacgctgacctgtttcgcaaacacgagatcgatggcaaggccctgctgctgctgcgcagtgacatgatgatgaagtacatgggcctgaagctggggcctgcactcaagctctcctaccacattgaccggctgaagcagggcaagttc

SEQ ID NO: 100 - protein, 250 aa, synthetic construct
  REGION 1..250: note = Description of sequence: KOX1(KRAB)-MeCP2(t)-SCMH1
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEVGSASAGSPGSDRYLESRDASRLSGRDPSSWTVEDVMQFVREADPQLGPHADLFRKHEIDGKALLLLRSDMMMKYMGLKLGPALKLSYHIDRLKQGKF

SEQ ID NO: 101 - DNA, 1404 bp, synthetic construct
  misc_feature 1..1404: note = Description of sequence: ZIM3(KRAB)-MeCP2-KOX1(KRAB)
  source 1..1404: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggcagcgcttctgctggacggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 102 - protein, 468 aa, synthetic construct
  REGION 1..468: note = Description of sequence: ZIM3(KRAB)-MeCP2-KOX1(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSASAGRTLVTFKDVFVDFTREEWKLLDTAQQILYRNVMLENYKNLVSLGYQLTKPDVILRLEKGEEPWLV

SEQ ID NO: 103 - DNA, 780 bp, synthetic construct
  misc_feature 1..780: note = Description of sequence: KOX1(KRAB)-MeCP2(t)-RYBP
  source 1..780: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtgggctctggcagcgcttctgctggaaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagaccgtcagcatcgaggtcaaggaagtgggcagcgcttctgctggagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatg

SEQ ID NO: 104 - protein, 260 aa, synthetic construct
  REGION 1..260: note = Description of sequence: KOX1(KRAB)-MeCP2(t)-RYBP
RTLVTFKDVFVDFTREEWKLLDTAQQILYRNVMLENYKNLVSLGYQLTKPDVILRLEKGEEPWLVGSGSASAGTTSTQVMVIKRPGRKRKAEADPQAIPKKRGRKPGSVVAAAAAEAKKKAVKESSIRSVQETVLPIKKRKTRETVSIEVKEVGSASAGDPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDM

SEQ ID NO: 105 - DNA, 1152 bp, synthetic construct
  misc_feature 1..1152: note = Description of sequence: KRBOX1(KRAB)-MAX-MGA
  source 1..1152: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggacagccgtcctgtactcacatctctgcagatgaaaaagcagctgaaaggagtcgaaaggctccaccaattcctctaaaactgaagcctgattactggagtgacaaactacagaaagaagcagaagcgtttgcttattatcgccggacacacactgccaatgagcggcggcggcgtggtgaaatgagggatctctttgagaaattaaagatcacattgggattacttcattcttccaaggtttccaaaagtctcattcttactcgagccttcagtgaaattcagggactaacagatcaggcagacaaattgataggacagaaaaatctcctgactcgaaaacggaatattctgatacggaaagta

SEQ ID NO: 106 - protein, 384 aa, synthetic construct
  REGION 1..384: note = Description of sequence: KRBOX1(KRAB)-MAX-MGA
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGQPSCTHISADEKAAERSRKAPPIPLKLKPDYWSDKLQKEAEAFAYYRRTHTANERRRRGEMRDLFEKLKITLGLLHSSKVSKSLILTRAFSEIQGLTDQADKLIGQKNLLTRKRNILIRKV

SEQ ID NO: 107 - DNA, 1101 bp, synthetic construct
  misc_feature 1..1101: note = Description of sequence: KRBOX1(KRAB)-MAX-ZNF264(KRAB)
  source 1..1101: mol_type = other DNA
atgacagctgtgtccttaacaaccaggccccaggaatcagtggcttttgaggacgtggctgtgtacttcactacgaaggaatgggccatcatggtgcctgccgagagggccttgtacagggatgtgatgctggagaactatgaggctgtggcctttgtagtgccacccacttccaaaccagctttggtctctcatctggagcaagggaaagagtcctgtttcacccagccacagggagtcctaagcaggaatgactggagagcaggctggggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggagcggcagcggtgctgacggaccgggcccaggtgtctgtgacctttgatgatgtggctgtgactttcaccaaggaggagtgggggcagctggacctagctcagcggaccctgtaccaggaggtgatgctggaaaactgtgggctcctggtgtctctggggtgtcctgttcccaaagctgagctgatctgccacctagagcatgggcaggagccatggaccaggaaggaagacctctcccaagacacctgtccaggcgacaaaggaaaacctaagaccacagaacctaccacttgtgagccagccttgtcagag

SEQ ID NO: 108 - protein, 367 aa, synthetic construct
  REGION 1..367: note = Description of sequence: KRBOX1(KRAB)-MAX-ZNF264(KRAB)
MTAVSLTTRPQESVAFEDVAVYFTTKEWAIMVPAERALYRDVMLENYEAVAFVVPPTSKPALVSHLEQGKESCFTQPQGVLSRNDWRAGWGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSE

SEQ ID NO: 109 - DNA, 1119 bp, synthetic construct
  misc_feature 1..1119: note = Description of sequence: ZIM3(KRAB)-MAX-ZIM3(KRAB)
  source 1..1119: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagcggcagcgcttctgctggaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctc

SEQ ID NO: 110 - protein, 373 aa, synthetic construct
  REGION 1..373: note = Description of sequence: ZIM3(KRAB)-MAX-ZIM3(KRAB)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEASGSASAGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 111 - DNA, 111 bp, synthetic construct
  misc_feature 1..111: note = Description of sequence: MeCP2(NID)
  source 1..111: mol_type = other DNA
gccaaaaagaaagccgtgaaggagtcttctatccgatctgtgcaggagaccgtactccccatcaagaagcgcaagacccgggagaccgtcagcatcgaggtcaaggaagtg

SEQ ID NO: 112 - protein, 37 aa, synthetic construct
  REGION 1..37: note = Description of sequence: MeCP2(NID)
AKKKAVKESSIRSVQETVLPIKKRKTRETVSIEVKEV

SEQ ID NO: 113 - DNA, 435 bp, synthetic construct
  misc_feature 1..435: note = Description of sequence: ZIM3(KRAB)-MeCP2(NID)
  source 1..435: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggagccaaaaagaaagccgtgaaggagtcttctatccgatctgtgcaggagaccgtactccccatcaagaagcgcaagacccgggagaccgtcagcatcgaggtcaaggaagtg

SEQ ID NO: 114 - protein, 145 aa, synthetic construct
  REGION 1..145: note = Description of sequence: ZIM3(KRAB)-MeCP2(NID)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGAKKKAVKESSIRSVQETVLPIKKRKTRETVSIEVKEV

SEQ ID NO: 115 - DNA, 22 bp, synthetic construct
  misc_feature 1..22: note = Description of sequence: IPO8 (forward)
  source 1..22: mol_type = other DNA
ggcatacagtttaacctgccac

SEQ ID NO: 116 - DNA, 23 bp, synthetic construct
  misc_feature 1..23: note = Description of sequence: IPO8 (reverse)
  source 1..23: mol_type = other DNA
caggagaggcatcatgtctgtaa

SEQ ID NO: 117 - DNA, 20 bp, synthetic construct
  misc_feature 1..20: note = Description of sequence: CANX(forward)
  source 1..20: mol_type = other DNA
gatccagacgcagagaaacc

SEQ ID NO: 118 - DNA, 20 bp, synthetic construct
  misc_feature 1..20: note = Description of sequence: CANX (reverse)
  source 1..20: mol_type = other DNA
catccaggagctgactcaca

SEQ ID NO: 119 - DNA, 20 bp, synthetic construct
  misc_feature 1..20: note = Description of sequence: SYVN1(forward)
  source 1..20: mol_type = other DNA
accagcatccctagctcaga

SEQ ID NO: 120 - DNA, 20 bp, synthetic construct
  misc_feature 1..20: note = Description of sequence: SYVN1 (reverse)
  source 1..20: mol_type = other DNA
tcctcaggcatctcctctgt

SEQ ID NO: 121 - DNA, 23 bp, synthetic construct
  misc_feature 1..23: note = Description of sequence: BLM(forward)
  source 1..23: mol_type = other DNA
cagactccgaaggaagttgtatg

SEQ ID NO: 122 - DNA, 23 bp, synthetic construct
  misc_feature 1..23: note = Description of sequence: BLM (reverse)
  source 1..23: mol_type = other DNA
tttggggtggtgtaacaaatgat

SEQ ID NO: 123 - DNA, 20 bp, synthetic construct
  misc_feature 1..20: note = Description of sequence: SEL1L(forward)
  source 1..20: mol_type = other DNA
gagggggaaagtgtcacaga

SEQ ID NO: 124 - DNA, 20 bp, synthetic construct
  misc_feature 1..20: note = Description of sequence: SEL1L (reverse)
  source 1..20: mol_type = other DNA
ggtcaaagctggtttccgta

SEQ ID NO: 125 - DNA, 21 bp, synthetic construct
  misc_feature 1..21: note = Description of sequence: DNAJC19(forward)
  source 1..21: mol_type = other DNA
agtggtagcagttggactgac

SEQ ID NO: 126 - DNA, 23 bp, synthetic construct
  misc_feature 1..23: note = Description of sequence: DNAJC19 (reverse)
  source 1..23: mol_type = other DNA
ggcagattttggtaggctttgaa

SEQ ID NO: 127 - DNA, 21 bp, synthetic construct
  misc_feature 1..21: note = Description of sequence: KRAS(forward)
  source 1..21: mol_type = other DNA
acagagagtggaggatgcttt

SEQ ID NO: 128 - DNA, 21 bp, synthetic construct
  misc_feature 1..21: note = Description of sequence: KRAS (reverse)
  source 1..21: mol_type = other DNA
tttcacacagccaggagtctt

SEQ ID NO: 129 - DNA, 21 bp, synthetic construct
  misc_feature 1..21: note = Description of sequence: MRPS11(forward)
  source 1..21: mol_type = other DNA
catccgagttgtggtgaaagg

SEQ ID NO: 130 - DNA, 22 bp, synthetic construct
  misc_feature 1..22: note = Description of sequence: MRPS11 (reverse)
  source 1..22: mol_type = other DNA
gattggggtgttgtctgtgatt

SEQ ID NO: 131 - RNA, 91 nt, synthetic construct
  misc_feature 1..91: note = Description of sequence: sgRNA scaffold
  source 1..91: mol_type = other RNA
gtttcagagctacagcagaaatgctgtagcaagttgaaataaggctagtccgttatcaacttgaaaaagtggcaccgagtcggtgcttttt

SEQ ID NO: 132 - RNA, 199 nt, synthetic construct
  misc_feature 1..199: note = Description of sequence: sgRNA scaffold w/ pp7 stem loop
  source 1..199: mol_type = other RNA
gtttaagagctatgctgcgaatacgagggagcagacgatatggcgtcgctcctctccacgagagcatatgggctccgtggtctcgtacaccatcagggtacgtatcagacaccatcagggtctgctcgtattcgcagcatagcaagtttaaataaggctagtccgttatcaacttgaaaaagtggcaccgagtcggtgc

SEQ ID NO: 133 - RNA, 21 nt, synthetic construct
  misc_feature 1..21: note = Description of sequence: Non-Targeting
  source 1..21: mol_type = other RNA
gaaatgtgagatcagagtaat

SEQ ID NO: 134 - RNA, 22 nt, synthetic construct
  misc_feature 1..22: note = Description of sequence: SV40-eGFP Reporter -229/301(T)
  source 1..22: mol_type = other RNA
gaaagtccccaggctccccagc

SEQ ID NO: 135 - RNA, 20 nt, synthetic construct
  misc_feature 1..20: note = Description of sequence: SV40-eGFP Reporter -43(NT)
  source 1..20: mol_type = other RNA
ctacttctggaatagctcag

SEQ ID NO: 136 - RNA, 20 nt, synthetic construct
  misc_feature 1..20: note = Description of sequence: SV40-eGFP Reporter -68(T)
  source 1..20: mol_type = other RNA
ctattccagaagtagtgagg

SEQ ID NO: 137 - RNA, 20 nt, synthetic construct
  misc_feature 1..20: note = Description of sequence: SV40-eGFP Reporter -134(NT)
  source 1..20: mol_type = other RNA
gccatggggcggagaatggg

SEQ ID NO: 138 - RNA, 20 nt, synthetic construct
  misc_feature 1..20: note = Description of sequence: SV40-eGFP Reporter -258(T)
  source 1..20: mol_type = other RNA
atctcaattagtcagcaacc

SEQ ID NO: 139 - RNA, 20 nt, synthetic construct
  misc_feature 1..20: note = Description of sequence: SV40-eGFP Reporter -349(NT)
  source 1..20: mol_type = other RNA
ctaactgacacactctagag

SEQ ID NO: 140 - RNA, 20 nt, synthetic construct
  misc_feature 1..20: note = Description of sequence: SV40-eGFP Reporter -313(T)
  source 1..20: mol_type = other RNA
ttagggtgtggaaagtcccc

SEQ ID NO: 141 - RNA, 20 nt, synthetic construct
  misc_feature 1..20: note = Description of sequence: SV40-eGFP Reporter +153(T)
  source 1..20: mol_type = other RNA
ctgaagttcatctgcaccac

SEQ ID NO: 142 - RNA, 20 nt, synthetic construct
  misc_feature 1..20: note = Description of sequence: SV40-eGFP Reporter +31(T)
  source 1..20: mol_type = other RNA
gggcgaggagctgttcaccg

SEQ ID NO: 143 - RNA, 20 nt, synthetic construct
  misc_feature 1..20: note = Description of sequence: CANX
  source 1..20: mol_type = other RNA
tcgggcctgtgaggacctcg

SEQ ID NO: 144 - RNA, 20 nt, synthetic construct
  misc_feature 1..20: note = Description of sequence: SYVN
  source 1..20: mol_type = other RNA
acacctcacttccggcggcg

SEQ ID NO: 145 - RNA, 20 nt, synthetic construct
  misc_feature 1..20: note = Description of sequence: BLM
  source 1..20: mol_type = other RNA
aggaaacggaagaacccgag

SEQ ID NO: 146 - RNA, 20 nt, synthetic construct
  misc_feature 1..20: note = Description of sequence: SEL1L
  source 1..20: mol_type = other RNA
atactgacccgaggacgccg

SEQ ID NO: 147 - RNA, 20 nt, synthetic construct
  misc_feature 1..20: note = Description of sequence: DNAJC19 sgRNA A
  source 1..20: mol_type = other RNA
gggatgagccgtgctcccgg

SEQ ID NO: 148 - RNA, 20 nt, synthetic construct
  misc_feature 1..20: note = Description of sequence: DNAJC19 sgRNA B
  source 1..20: mol_type = other RNA
gggcgcctgtgcttgaggtt

SEQ ID NO: 149 - RNA, 20 nt, synthetic construct
  misc_feature 1..20: note = Description of sequence: DNAJC19 sgRNA C
  source 1..20: mol_type = other RNA
ggtgctgtgaagatgtgtta

SEQ ID NO: 150 - RNA, 21 nt, synthetic construct
  misc_feature 1..21: note = Description of sequence: KRAS sgRNA A
  source 1..21: mol_type = other RNA
gcggcgaaggtggcggcggct

SEQ ID NO: 151 - RNA, 20 nt, synthetic construct
  misc_feature 1..20: note = Description of sequence: KRAS sgRNA B
  source 1..20: mol_type = other RNA
ggcagtggcggcggcgaagg

SEQ ID NO: 152 - RNA, 20 nt, synthetic construct
  misc_feature 1..20: note = Description of sequence: KRAS sgRNA C
  source 1..20: mol_type = other RNA
gctcccagtccgaaatggcg

SEQ ID NO: 153 - RNA, 20 nt, synthetic construct
  misc_feature 1..20: note = Description of sequence: MRPS11 sgRNA A
  source 1..20: mol_type = other RNA
gctgcagacggaaactgact

SEQ ID NO: 154 - RNA, 20 nt, synthetic construct
  misc_feature 1..20: note = Description of sequence: MRPS11 sgRNA B
  source 1..20: mol_type = other RNA
ggggtcaattcaagtcatgc

SEQ ID NO: 155 - RNA, 20 nt, synthetic construct
  misc_feature 1..20: note = Description of sequence: MRPS11 sgRNA C
  source 1..20: mol_type = other RNA
gtggctcaagggacacgagt

SEQ ID NO: 156 - DNA, 108 bp, synthetic construct
  misc_feature 1..108: note = Description of sequence: MeCP2(NLS 1X)
  source 1..108: mol_type = other DNA
accacatccacccaggtcatggtgatcaaacgccccggcaggaagcgaaaagctgaggccgaccctcaggccattcccaagaaacggggccgaaagccggggagtgtg

SEQ ID NO: 157 - protein, 36 aa, synthetic construct
  REGION 1..36: note = Description of sequence: MeCP2(NLS 1X)
TTSTQVMVIKRPGRKRKAEADPQAIPKKRGRKPGSV

SEQ ID NO: 158 - DNA, 432 bp, synthetic construct
  misc_feature 1..432: note = Description of sequence: ZIM3(KRAB)-MeCP2(NLS 1X)
  source 1..432: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaccacatccacccaggtcatggtgatcaaacgccccggcaggaagcgaaaagctgaggccgaccctcaggccattcccaagaaacggggccgaaagccggggagtgtg

SEQ ID NO: 159 - protein, 144 aa, synthetic construct
  REGION 1..144: note = Description of sequence: ZIM3(KRAB)-MeCP2(NLS 1X)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGTTSTQVMVIKRPGRKRKAEADPQAIPKKRGRKPGSV

SEQ ID NO: 160 - DNA, 240 bp, synthetic construct
  misc_feature 1..240: note = Description of sequence: MeCP2(NLS 2X)
  source 1..240: mol_type = other DNA
accacatccacccaggtcatggtgatcaaacgccccggcaggaagcgaaaagctgaggccgaccctcaggccattcccaagaaacggggccgaaagccggggagtgtgggatctgggaaatctgggtcaggaaccacatccacccaggtcatggtgatcaaacgccccggcaggaagcgaaaagctgaggccgaccctcaggccattcccaagaaacggggccgaaagccggggagtgtg

SEQ ID NO: 161 - protein, 80 aa, synthetic construct
  REGION 1..80: note = Description of sequence: MeCP2(NLS 2X)
TTSTQVMVIKRPGRKRKAEADPQAIPKKRGRKPGSVGSGKSGSGTTSTQVMVIKRPGRKRKAEADPQAIPKKRGRKPGSV

SEQ ID NO: 162 - DNA, 564 bp, synthetic construct
  misc_feature 1..564: note = Description of sequence: ZIM3(KRAB)-MeCP2(NLS 2X)
  source 1..564: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaccacatccacccaggtcatggtgatcaaacgccccggcaggaagcgaaaagctgaggccgaccctcaggccattcccaagaaacggggccgaaagccggggagtgtgggatctgggaaatctgggtcaggaaccacatccacccaggtcatggtgatcaaacgccccggcaggaagcgaaaagctgaggccgaccctcaggccattcccaagaaacggggccgaaagccggggagtgtg

SEQ ID NO: 163 - protein, 188 aa, synthetic construct
  REGION 1..188: note = Description of sequence: ZIM3(KRAB)-MeCP2(NLS 2X)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGTTSTQVMVIKRPGRKRKAEADPQAIPKKRGRKPGSVGSGKSGSGTTSTQVMVIKRPGRKRKAEADPQAIPKKRGRKPGSV

SEQ ID NO: 164 - DNA, 21 bp, synthetic construct
  misc_feature 1..21: note = Description of sequence: SV40NLS
  source 1..21: mol_type = other DNA
ccgaaaaagaagcgtaaggtt

SEQ ID NO: 165 - protein, 7 aa, synthetic construct
  REGION 1..7: note = Description of sequence: SV40NLS
PKKKRKV

SEQ ID NO: 166 - DNA, 345 bp, synthetic construct
  misc_feature 1..345: note = Description of sequence: ZIM3(KRAB)-SV40NLS
  source 1..345: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaccgaaaaagaagcgtaaggtt

SEQ ID NO: 167 - protein, 115 aa, synthetic construct
  REGION 1..115: note = Description of sequence: ZIM3(KRAB)-SV40NLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGPKKKRKV

SEQ ID NO: 168 - DNA, 54 bp, synthetic construct
  misc_feature 1..54: note = Description of sequence: BPSV40NLS
  source 1..54: mol_type = other DNA
aaacggacagccgacggaagcgagttcgagtcaccaaagaagaagcggaaagtc

SEQ ID NO: 169 - protein, 18 aa, synthetic construct
  REGION 1..18: note = Description of sequence: BPSV40NLS
KRTADGSEFESPKKKRKV

SEQ ID NO: 170 - DNA, 378 bp, synthetic construct
  misc_feature 1..378: note = Description of sequence: ZIM3(KRAB)-BPSV40NLS
  source 1..378: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaaacggacagccgacggaagcgagttcgagtcaccaaagaagaagcggaaagtc

SEQ ID NO: 171 - protein, 126 aa, synthetic construct
  REGION 1..126: note = Description of sequence: ZIM3(KRAB)-BPSV40NLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGKRTADGSEFESPKKKRKV

SEQ ID NO: 172 - DNA, 78 bp, synthetic construct
  misc_feature 1..78: note = Description of sequence: vBPSV40NLS
  source 1..78: mol_type = other DNA
aaaagaaccgccgacggcagcgagaagcgcaccgccgacagccagcacagcaccccccccaagaccaagcgcaaggtg

SEQ ID NO: 173 - protein, 26 aa, synthetic construct
  REGION 1..26: note = Description of sequence: vBPSV40NLS
KRTADGSEKRTADSQHSTPPKTKRKV

SEQ ID NO: 174 - DNA, 402 bp, synthetic construct
  misc_feature 1..402: note = Description of sequence: ZIM3(KRAB)-vBPSV40NLS
  source 1..402: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaaaagaaccgccgacggcagcgagaagcgcaccgccgacagccagcacagcaccccccccaagaccaagcgcaaggtg

SEQ ID NO: 175 - protein, 134 aa, synthetic construct
  REGION 1..134: note = Description of sequence: ZIM3(KRAB)-vBPSV40NLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGKRTADGSEKRTADSQHSTPPKTKRKV

SEQ ID NO: 176 - DNA, 27 bp, synthetic construct
  misc_feature 1..27: note = Description of sequence: cMYCNLS
  source 1..27: mol_type = other DNA
cccgccgccaagcgcgtgaagctggac

SEQ ID NO: 177 - protein, 9 aa, synthetic construct
  REGION 1..9: note = Description of sequence: cMYCNLS
PAAKRVKLD

SEQ ID NO: 178 - DNA, 351 bp, synthetic construct
  misc_feature 1..351: note = Description of sequence: ZIM3(KRAB)-cMYCNLS
  source 1..351: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggacccgccgccaagcgcgtgaagctggac

SEQ ID NO: 179 - protein, 117 aa, synthetic construct
  REGION 1..117: note = Description of sequence: ZIM3(KRAB)-cMYCNLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGPAAKRVKLD

SEQ ID NO: 180 - DNA, 48 bp, synthetic construct
  misc_feature 1..48: note = Description of sequence: NucNLS
  source 1..48: mol_type = other DNA
aaacgccctgccgcaacgaagaaggctggacaggcgaaaaagaagaag

SEQ ID NO: 181 - protein, 16 aa, synthetic construct
  REGION 1..16: note = Description of sequence: NucNLS
KRPAATKKAGQAKKKK

SEQ ID NO: 182 - DNA, 372 bp, synthetic construct
  misc_feature 1..372: note = Description of sequence: ZIM3(KRAB)-NucNLS
  source 1..372: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaaacgccctgccgcaacgaagaaggctggacaggcgaaaaagaagaag

SEQ ID NO: 183 - protein, 124 aa, synthetic construct
  REGION 1..124: note = Description of sequence: ZIM3(KRAB)-NucNLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGKRPAATKKAGQAKKKK

SEQ ID NO: 184 - DNA, 123 bp, synthetic construct
  misc_feature 1..123: note = Description of sequence: vBPSV40NLS-SV40NLS
  source 1..123: mol_type = other DNA
aaaagaaccgccgacggcagcgagaagcgcaccgccgacagccagcacagcaccccccccaagaccaagcgcaaggtgggatctgggaaatctgggtcaggaccgaaaaagaagcgtaaggtt

SEQ ID NO: 185 - protein, 41 aa, synthetic construct
  REGION 1..41: note = Description of sequence: vBPSV40NLS-SV40NLS
KRTADGSEKRTADSQHSTPPKTKRKVGSGKSGSGPKKKRKV

SEQ ID NO: 186 - DNA, 447 bp, synthetic construct
  misc_feature 1..447: note = Description of sequence: ZIM3(KRAB)- vBPSV40NLS-SV40NLS
  source 1..447: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaaaagaaccgccgacggcagcgagaagcgcaccgccgacagccagcacagcaccccccccaagaccaagcgcaaggtgggatctgggaaatctgggtcaggaccgaaaaagaagcgtaaggtt

SEQ ID NO: 187 - protein, 149 aa, synthetic construct
  REGION 1..149: note = Description of sequence: ZIM3(KRAB)- vBPSV40NLS-SV40NLS
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGKRTADGSEKRTADSQHSTPPKTKRKVGSGKSGSGPKKKRKV

SEQ ID NO: 188 - DNA, 180 bp, synthetic construct
  misc_feature 1..180: note = Description of sequence: vBPSV40NLS(2X)
  source 1..180: mol_type = other DNA
aaaagaaccgccgacggcagcgagaagcgcaccgccgacagccagcacagcaccccccccaagaccaagcgcaaggtgggatctgggaaatctgggtcaggaaaaagaaccgccgacggcagcgagaagcgcaccgccgacagccagcacagcaccccccccaagaccaagcgcaaggtg

SEQ ID NO: 189 - protein, 60 aa, synthetic construct
  REGION 1..60: note = Description of sequence: vBPSV40NLS(2X)
KRTADGSEKRTADSQHSTPPKTKRKVGSGKSGSGKRTADGSEKRTADSQHSTPPKTKRKV

SEQ ID NO: 190 - DNA, 504 bp, synthetic construct
  misc_feature 1..504: note = Description of sequence: ZIM3(KRAB)- vBPSV40NLS(2X)
  source 1..504: mol_type = other DNA
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccgtggttggaggaagaggaagtgctgggaagtggccgtgcagaaaaaaatggggacattggagggcagatttggaagccaaaggatgtgaaagagagtctcggctctggcagcgcttctgctggaaaaagaaccgccgacggcagcgagaagcgcaccgccgacagccagcacagcaccccccccaagaccaagcgcaaggtgggatctgggaaatctgggtcaggaaaaagaaccgccgacggcagcgagaagcgcaccgccgacagccagcacagcaccccccccaagaccaagcgcaaggtg

SEQ ID NO: 191 - protein, 168 aa, synthetic construct
  REGION 1..168: note = Description of sequence: ZIM3(KRAB)- vBPSV40NLS(2X)
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSGSASAGKRTADGSEKRTADSQHSTPPKTKRKVGSGKSGSGKRTADGSEKRTADSQHSTPPKTKRKV